(19) United States
(12) Patent Application Publication
(10) Pub. No.: US 2023/0187067 A1
(43) Pub. Date: Jun. 15, 2023

(54) USE OF CLINICAL PARAMETERS FOR THE PREDICTION OF SIRS

(71) Applicant: Peach IntelliHealth, Inc., New York, NY (US)

(72) Inventors: L.S. Klaudyne Hong, New York, NY (US); Gerald Wogan, Belmont, MA (US); Luigi Vacca, Weston, MA (US); Bruce Tidor, Lexington, MA (US)

(21) Appl. No.: 16/085,929

(22) PCT Filed: Mar. 23, 2017
(86) PCT No.: PCT/US2017/023885

§ 371 Date: Sep. 17, 2018

Related U.S. Application Data
(60) Provisional application No. 62/312,339, filed on Mar. 23, 2016.

(51) Int. Cl. G16H 50/20; G16H 50/30; G16H 50/70

(52) U.S. Cl. CPC G16H 50/20; G16H 50/30; G16H 50/70

(57) ABSTRACT

A system for disease prediction includes processing circuitry configured to receive a dataset including data of a pa -tient population, the data including for each of a plurality of patients of the patient population, values for a plurality of features, and a diagnosis value indicating whether a disease has been diagnosed. The processing circuitry is configured to, based on correlations between the values, select from the dataset a plurality of subsets of the features, and, for each of at least one of the subsets, execute a machine learning process with the respective subset and the diagnosis values as input parameters, the execution generating a respective prediction model. The processing circuitry is configured to output the respective prediction model.

### FIELD OF THE INVENTION

The present invention relates to the composition and use of clinical parameters for the prediction, or risk stratification for Systemic Inflammatory Response Syndrome (SIRS) several hours to days before SIRS symptoms are observable for a definitive diagnosis in a patient. The ability to predict the onset of SIRS, prior to the appearance of clinical symptoms, enables physicians to initiate therapy in an expeditious manner, thereby improving outcomes. This applies to patients that have non-infectious SIRS or patients with SIRS that progress to sepsis. The present invention is also directed to a method of determining parameters and combinations thereof, which are relevant for predicting onset of a disease, e.g., SIRS.

### DISCUSSION

A biomarker is a measurable substance in an organism whose presence is indicative of some phenomenon such as disease, infection, or environmental exposure. For example, detection of a cancer-associated protein biomarker in the blood means the patient already has cancer. Pursuant to this invention, however, a combination of clinical features or parameters such as physiologic and/or clinical procedures (e.g., PO2 or Fingerstick Glucose) is used to predict how likely the patient will progress to SIRS. These features are noted as part of a patient’s health records, but are not previously associated with SIRS prior to this invention.

Prior published work related to the application of artificial intelligence and/or biomarker approaches to sepsis was designed mainly to improve the sensitivity and specificity of sepsis diagnosis at various stages of the progressive syndrome. Thus, the studies involved were conducted in patients, mainly in intensive care units, for whom a diagnosis of sepsis had already been made, based on widely accepted clinical criteria. In contrast, the invention predicts the onset of SIRS, prior to the appearance of clinical symptoms, which the invention has accomplished in intensive care patients with a sensitivity of 85-95%, an accuracy of 80-85%, and area under the curve (AUC) of 0.70-0.85. One of ordinary skill in the art would readily understand the meaning of the foregoing terms, which are standard in the machine learning literature and are well known to one of ordinary skill in the art. The present invention advantageously uses algorithms to analyze the types of available clinical and laboratory data that are normally collected in hospital patients to make its predictions, without requiring blood sampling and analysis for specific biomarkers.

### SIRS

SIRS, Systemic Inflammatory Response Syndrome, is a whole-body inflammatory state. A mild systemic inflammatory response to any bodily insult may normally have some salutatory effects. However, a marked or prolonged response, such as that associated with severe infections, is often deleterious and can result in widespread organ dysfunction. Many infectious agents are capable of inducing SIRS. These organisms either elaborate toxins or stimulate release of substances that trigger this response. Commonly recognized initiators are the lipopolysaccharides (LPSs, sometimes referred to as endotoxin), that are released by gram-negative bacteria. The resulting response involves a complex interaction between macrophages/monocytes, neutrophils, lymphocytes, platelets, and endothelial cells that can affect nearly every organ. Infectious SIRS can occur as a result of the following pathologic conditions: bacterial sepsis; burn and wound infections; candidiasis; cellulitis; cholecystitis; pneumonia; diabetic foot infection; infective endocarditis; influenza; intra-abdominal infections (e.g., diverticulitis, appendicitis); meningitis; colitis; pyelonephritis; septic arthritis; toxic shock syndrome; and urinary tract infections.

While SIRS can lead to sepsis, SIRS is not exclusively related to infection. Its etiology is broad and includes noninfectious conditions, surgical procedures, trauma, medications, and therapies. Some examples of conditions associated with non-infectious SIRS include: acute mesenteric ischemia; adrenal insufficiency; autoimmune disorders; burns; chemical aspiration; cirrhosis; dehydration; drug reaction; electrical injuries; hemorrhagic shock; hematologic malignancy; intestinal perforation; medication side effect; myocardial infarction; pancreatitis; seizure; substance abuse; surgical procedures; transfusion reactions; upper gastrointestinal bleeding; and vasculitis.

SIRS has been clinically defined as the simultaneous presence of two or more of the following features in adults: body temperature >38° C. (100.4° F.) or <36° C. (96.8° F.); heart rate of >90 beats per minute; respiratory rate of >20 breaths per minute or arterial carbon dioxide tension (PₐCO₂) of <32 mm Hg; and abnormal white blood cell count (>12,000/µL or < 4,000/µL or >10% immature [band] forms).

### Pathophysiology of SIRS

The complex pathophysiology of SIRS is independent of etiologic factors, with minor differences with respect to the cascades that it incites. This pathophysiology is briefly outlined as follows. Inflammation, the body’s response to nonspecific insults that arise from chemical, traumatic, or infectious stimuli is a critically important component. The inflammation itself is a process involving humoral and cellular responses, complement, and cytokine cascades. The relationship between these complex interactions and SIRS has been defined as a three-stage process. See Bone et al. (1992) (all citations refer to references listed at the end of the document).

In stage 1, following an insult cytokines are produced at the site. Local cytokine production incites an inflammatory response, thereby promoting wound repair and recruitment of the reticular endothelial (fixed macrophage) system. This process is essential for normal host defense homeostasis, and its malfunction is life-threatening. Local inflammation, such as in the skin and subcutaneous soft tissues, carries the classic description of rubor (redness), tumor (swelling), dolor (pain), calor (increased heat) and functio laesa (loss of function). Importantly, on a local level, this cytokine and chemokine release may cause local tissue destruction or cellular injury by attracting activated leukocytes to the region.

In stage 2, small quantities of local cytokines are released into the circulation, enhancing the local response. This leads to growth factor stimulation and the recruitment of macrophages and platelets. This acute phase response is typically well-controlled by a decrease in pro-inflammatory mediators and by the release of endogenous antagonists.

In stage 3, a significant systemic reaction occurs if the inflammatory stimuli continue to spread into the systemic circulation. The cytokine release leads to destruction rather than protection. A consequence of this is the activation of numerous humoral cascades, generalized activation of the reticular endothelial system, and subsequent loss of circulatory integrity. This leads to end-organ dysfunction.

When SIRS is mediated by an infectious insult, the inflammatory cascade is often initiated by endotoxin. Tissue macrophages, monocytes, mast cells, platelets, and endothelial cells are able to produce a multitude of cytokines. The cytokines tissue necrosis factor-alpha (TNF-α) and interleukin-1 (IL-1) are released first and initiate several downstream cascades.

The release of IL-1 and TNF-α (or the presence of endotoxin) leads to cleavage of the nuclear factor NF-kappa B (NF-κB) inhibitor. Once the inhibitor is removed, NF-κB initiates expression of mRNAs encoding genes regulating production of other pro-inflammatory cytokines, primarily IL-6, IL-8, and interferon gamma. TNF- α and IL-1 have been shown to be released in large quantities within 1 hour of an insult and have both local and systemic effects. TNF-α and IL-1 are responsible for fever and the release of stress hormones (norepinephrine, vasopressin, activation of the renin-angiotensin-aldosterone system).

Other cytokines, especially IL-6, stimulate the release of acute-phase reactants such as C-reactive protein (CRP) and procalcitonin. Notably, infection has been shown to induce a greater release of TNF-α, thus inducing a greater release of IL-6 and IL-8 than trauma does. This is suggested to be the reason higher fever is associated with infection rather than trauma.

The pro-inflammatory interleukins either function directly on tissue or via secondary mediators to activate the coagulation cascade and the complement cascade as well as the release of nitric oxide, platelet-activating factor, prostaglandins, and leukotrienes. HMGB1 (high mobility group box 1) is a protein present in the cytoplasm and nuclei in a majority of cell types. It acts as a potent pro-inflammatory cytokine and is involved in delayed endotoxin lethality and sepsis. In response to infection or injury, as is seen with SIRS, HMGB1 is secreted by innate immune cells and/or released passively by damaged cells. Thus, elevated serum and tissue levels of HMGB1 are induced by many of the agents that cause SIRS.

A correlation that exists between inflammation and coagulation is critical to the progression of SIRS. IL-1 and TNF-α directly affect endothelial surfaces, leading to the expression of tissue factor. Tissue factor initiates the production of thrombin, thereby promoting coagulation, and is a pro-inflammatory mediator itself. Fibrinolysis is impaired by IL-1 and TNF-α via production of plasminogen activator inhibitor-1. Pro-inflammatory cytokines also disrupt the naturally occurring anti-inflammatory mediators, anti-thrombin and activated protein-C (APC). If unchecked, this coagulation cascade leads to complications resulting from microvascular thrombosis, including organ dysfunction. The complement system also plays a role in the coagulation cascade. Infection-related pro-coagulant activity is generally more severe than that produced by trauma.

The cumulative effect of this inflammatory cascade is an unbalanced state, with inflammation and coagulation dominating. To counteract the acute inflammatory response, the body is equipped to reverse this process via the counter-inflammatory response syndrome (CARS). IL-4 and IL-10 are cytokines responsible for decreasing the production of TNF-α, IL-1, IL-6, and IL-8. The acute phase response also produces antagonists to TNF-α and IL-1 receptors. These antagonists either bind the cytokine, and thereby inactivate it, or block the receptors. The balance of SIRS and CARS helps to determine a patient’s outcome after an insult.

The normal physiology of an inflammatory response consists of an acute pro-inflammatory state resulting from innate immune system recognition of ligands, and an anti-inflammatory phase that can serve to modulate the pro-inflammatory phase. Under normal circumstances, these coordinated responses direct a return to homeostasis. Severe or protracted SIRS can result in septic shock. Bacteremia is usually present but may be absent. Increased nitric oxide levels may be responsible for vasodilation, and hypotension is also due to decreased circulating intravascular volume resulting from diffuse capillary leaks. Activation of platelets and the coagulation cascade can lead to the formation of fibrin- platelet aggregates, which further compromise tissue blood flow. The release of vasoactive substances, formation of microthrombi in the pulmonary circulation, or both together increase pulmonary vascular resistance, whereas systemic venodilation and transudation of fluid into tissues result in relative hypovolemia.

### Epidemiology of SIRS

The true incidence of SIRS is unknown but probably much higher than documented, owing to the nonspecific nature of its definition. Not all patients with SIRS require hospitalization or have diseases that progress to serious illness. Because SIRS criteria are nonspecific and occur in patients who present with conditions ranging from influenza to cardiovascular collapse associated with severe pancreatitis, it is useful to stratify any incidence figures based on SIRS severity.

Results of epidemiologic studies conducted in the US have been published. A prospective survey of patients admitted to a tertiary care center revealed that 68% of hospital admissions to surveyed units met SIRS criteria. See Rangel-Fausto et al. (1995). The incidence of SIRS increased as the level of unit acuity increased. The following progression of patients with SIRS was noted: 26% developed sepsis, 18% developed severe sepsis, and 4% developed septic shock within 28 days of admission.

A hospital survey of SIRS revealed an overall in-hospital incidence of 542 episodes per 1000 hospital days. See Pittet et al. (1995). In comparison, the incidence in the intensive care unit (ICU) was 840 episodes per 1000 hospital days. Another study demonstrated that 62% of patients who presented to the emergency department with SIRS had a confirmed infection, while 38% did not. See Comstedt et al. (2009). Still, the incidence of severe SIRS associated with infection was found to be 3 cases per 1,000 population, or 2.26 cases per 100 hospital discharges. See Angus et al. (2001). The real incidence of SIRS, therefore, must be much higher and depends significantly on the rigor with which the definition is applied.

### Prognosis of SIRS Patients

Prognosis depends on the etiologic source of SIRS, as well as on associated comorbidities. A study of SIRS in acutely hospitalized medical patients demonstrated a 6.9 times higher 28-day mortality in SIRS patients than in non-SIRS patients. Most deaths occurred in SIRS patients with an associated malignancy. See Comstedt et al. (2009). Mortality rates in the study of tertiary care patients mentioned above, see Rangel-Fausto et al. (1995), were 7% (SIRS), 16% (sepsis), 20% (severe sepsis), and 46% (septic shock). The median time interval from SIRS to sepsis was inversely related to the number of SIRS criteria met. Morbidity was related to the causes of SIRS, complications of organ failure, and the potential for prolonged hospitalization. A study evaluating mortality in patients with suspected infection in the emergency department showed the following in-hospital mortality rates: Suspected infection without SIRS, 2.1%; Sepsis, 1.3%; Severe Sepsis, 9.2%; and Septic Shock, 28%. See Shapiro et al. (2006).

Evaluation of the SIRS criteria in patients who underwent transcatheter aortic valve implantation (TAVI) revealed that SIRS appeared to be a strong predictor of mortality. See Sinning et al. (2012). The occurrence of SIRS was characterized by a significantly elevated release of IL-6 and IL-8, with subsequent increase in the leukocyte count, C-reactive protein (CRP), and pro-calcitonin. The occurrence of SIRS was related to 30-day and 1-year mortality (18% vs 1.1% and 52.5% vs 9.9%, respectively) and independently predicted 1-year mortality risk.

The early identification and administration of supportive care is key in the management of patients with SIRS who could progress to Sepsis, Severe Sepsis or Septic Shock. Several studies have shown that fluids and antibiotics, when administered early in the disease process, can prevent hypoxemia and hypotension. See Annane et al. (2005); Dellinger et al. (2008); Hollenberg et al. (2004); and Dellinger et al. (2013). Indeed, international guidelines on the management of sepsis recommend the initiation of resuscitative measures within 6 hours of the recognition of septic symptoms. See Dellinger et al. (2013).

The ability to predict the onset of SIRS, prior to the appearance of clinical symptoms would enable physicians to initiate therapy in an expeditious manner, thereby improving outcomes. This applies to patients that have non-infectious SIRS or patients with SIRS that progress to Sepsis.

SIRS is associated with a variety of inflammatory states, including sepsis, pancreatitis, burns, surgery, etc. When confronted with SIRS, physicians typically attempt to identify potential etiologies and interventions that can prevent adverse outcomes. For example, sepsis is a frequently encountered problem in intensive care unit (ICU) patients who have been instrumented with invasive catheters. Since SIRS precedes sepsis, and the development of sepsis is associated with significant morbidity and mortality, the presence of SIRS in the ICU cannot be ignored. SIRS in these patients often prompts a search for a focus of infection and potentially the administration of empiric antibiotics. Since minimizing the time to antibiotic administration is one intervention that has consistently been shown to improve outcomes in these patients, SIRS often serves as an alarm that causes health care workers to consider the use of antimicrobials in selected patients.

However, using the invention to predict the onset of SIRS 6 to 48 hours earlier (e.g., 6, 12, 24 or 48 hours earlier) would allow one to administer antibiotics earlier, with advantages either because the patients would not get as sick initially, before they get better, or because there is time to try one more antibiotic if the first one or two (or more) do not work. In patients with bacteremia, SIRS often portends the development of sepsis, severe sepsis and/or septic shock. It is important to recognize that in these patients SIRS is diagnosed after the patient has already been infected. Methods that identify patients who will eventually develop SIRS are desirable because they detect patients who are at an earlier stage in the infectious process. The key benefit of early and accurate SIRS prediction is the ability to identify patients who are at risk of infection before the infection has started to manifest itself. Since there are a great deal of data to suggest that the earlier supportive therapy is administered (e.g., fluid and antibiotics), the better the outcomes, a SIRS prediction prior to the onset of symptoms could significantly impact clinical management and outcomes. More precisely, the accurate prediction of SIRS 6 to 48 (e.g., 6, 12, 24 or 48) hours prior to the onset of symptoms would provide enough time to mobilize hospital resources, creating the best environment for the patient. For example:

Patients on inpatient floors who are identified as being at high risk of SIRS could be transferred to high acuity units that have a higher nurse-to-patient ratio, thereby helping to ensure that such patients are monitored in a manner that is commensurate with their risk;

A positive SIRS prediction in patients who are instrumented with invasive catheters (which on its own may increase one’s risk of bacteremia) would warrant closer monitoring for septic signs, and potentially a search for a septic focus. The threshold for the administration of fluids and empiric antibiotics in these patients would be significantly lower than patients who have not been identified as high risk; and

Patients who are identified as being at high risk for SIRS would benefit from a careful review of their medication history to ensure that they are not on agents that may be associated with a drug reaction (a cause of non-infectious SIRS). Careful review of medications in such patients provides one mechanism to circumvent adverse medication side effects.

### Biomarkers in the Diagnosis of SIRS

The role of biomarkers in the diagnosis of sepsis and patient management has been evaluated. See Bernstein (2008). SIRS is an acute response to trauma, burn, or infectious injury characterized by fever, hemodynamic and respiratory changes, and metabolic changes, not all of which are consistently present. The SIRS reaction involves hormonally driven changes in liver glycogen reserves, triggering of lipolysis, lean body proteolysis, and reprioritization of hepatic protein synthesis with up-regulation of synthesis of acute phase proteins and down-regulation of albumin and important circulating transport proteins. Understanding of the processes has led to the identification of biomarkers for identification of sepsis and severe, moderate or early SIRS, which also can hasten treatment and recovery. The SIRS reaction unabated leads to a recurring cycle with hemodynamic collapse from septic shock, indistinguishable from cardiogenic shock, and death.

By focusing on early and accurate diagnosis of infection in patients suspected of SIRS, antibiotic overuse and its associated morbidity and mortality may be avoided, and therapeutic targets may be identified. The performance of diagnostic algorithms and biomarkers for sepsis in patients presenting with leukocytosis and other findings has been evaluated. Suspected patients are usually identified by WBC above 12,000/Ml, procalcitonin level, SIRS and other criteria, such as serum biomarkers of sepsis. In a study of 435 patients, see Gultepe et al. (2014), procalcitonin alone was a superior marker for sepsis. In patients with sepsis there was a marked increase in procalcitonin (p = 0.0001), and in patients requiring ICU admission, heart rate and blood pressure monitoring, and assisted ventilation were increased (p = 0.0001).

The emergence of large-scale data integration in electronic health records (EHR) presents unprecedented opportunities for design of methods to construct knowledge from heterogeneous datasets, and as an extension, to inform clinical decisions. However, current ability to efficiently extract informed decision support is limited due to the complexity of the clinical states and decision process, missing data and lack of analytical tools to advise based on statistical relationships. A machine learning basis for a clinical decision support system to identify patients at high risk for hyperlactatemia based upon routinely measured vital signs and laboratory studies has been developed. See Gultepe et al. (2014).

Electronic health records of 741 adult patients who met at least two systemic inflammatory response syndrome (SIRS) criteria were used to associate patients’ vital signs, white blood cell count (WBC), with sepsis occurrence and mortality. Generative and discriminative classification (naive Bayes, support vector machines, Gaussian mixture models, hidden Markov models) were used to integrate heterogeneous patient data and form a predictive tool for the inference of lactate level and mortality risk.

An accuracy of 0.99 and discriminability of 1.00 area under the receiver operating characteristic curve (AUC) for lactate level prediction was obtained when the vital signs and WBC measurements were analyzed in a 24 h time bin. An accuracy of 0.73 and discriminability of 0.73 AUC for mortality prediction in patients with sepsis was achieved with three properties: median of lactate levels, mean arterial pressure, and median absolute deviation of the respiratory rate. These findings introduce a new scheme for the prediction of lactate levels and mortality risk from patient vital signs and WBC. Accurate prediction of both these variables can drive the appropriate response by clinical staff. See Gultepe et al. (2014).

### Sepsis

Sepsis is one of the oldest syndromes in medicine. It is the leading cause of death in non-coronary ICUs in the US, with associated mortality rates upwards of 80%. See Shapiro et al. (2006); Sinning et al. (2012); and Nierhaus et al. (2013). The term Sepsis refers to a clinical spectrum of complications, often starting with an initial infection. Untreated, the disease cascade progresses through stages with increasing mortality, from SIRS to Sepsis to Severe Sepsis to Septic Shock, and ultimately death. See Shapiro et al. (2006); Sinning et al. (2012); Nierhaus et al. (2013); and Lai et al. (2010). A representative course is illustrated in a prospective study that found 36% mortality in ICU patients with Sepsis, 52% in patients with Severe Sepsis and 82% in patients with Septic Shock. See Jekarl et al. (2013). While early goal-directed therapy has been shown to provide substantial benefits in patient outcomes, efficacy is contingent upon early detection or suspicion of the underlying septic etiology.

In 1992, an international consensus panel defined sepsis as a systemic inflammatory response to infection, noting that sepsis could arise in response to multiple infectious causes. The panel proposed the term “severe sepsis” to describe instances in which sepsis is complicated by acute organ dysfunction, and they codified “septic shock” as sepsis complicated by either hypotension that is refractory to fluid resuscitation or by hyperlactatemia. In 2003, a second consensus panel endorsed most of these concepts, with the caveat that signs of SIRS, such as tachycardia or an elevated white-cell count, occur in many infectious and noninfectious conditions and therefore are not helpful in distinguishing sepsis from other conditions. Thus, “severe sepsis” and “sepsis” are sometimes used interchangeably to describe the syndrome of infection complicated by acute organ dysfunction. See Angus et al. (2013).

These definitions have achieved widespread usage and become the gold standard in sepsis protocols and research. Yet sepsis clearly comprises a complex, dynamic, and relational distortion of human life. Given the profound scope of the loss of life worldwide, a need has been expressed to disengage from the simple concepts of the past and develop new approaches which engage sepsis in its true form, as a complex, dynamic, and relational pattern of death. See Lawrence A. Lynn (2014).

### Biomarkers in Diagnosis of Sepsis

Several molecular markers have been discussed to facilitate diagnosis and treatment monitoring of sepsis in humans and several animal species. The most widely used ones may be CRP (C-reactive protein) and PCT (procalcitonin). Also various interleukins have been discussed as potential biomarkers of sepsis. However they are of limited use at present because of a lack of specificity. For example, Carrigan et al. (2004) reported that sensitivities and specificities for these markers in humans, in whom septic disease patterns have been extensively investigated, sensitivity and specificity of current markers can (even as mean values) be as low as 33% and 66%, respectively. Published data also have a high degree of inhomogeneity. Thus, there is a definite need for new diagnostic markers with improved diagnostic characteristics for the diagnosis of sepsis, especially early diagnosis. In systemic inflammation, i.e. in multiply traumatized patients, such a diagnosis is often very difficult because of other pathological processes interfering with the “normal” physiological values and parameters measured in standard intensive care medicine. Diagnosis of sepsis in patients with systemic inflammation, e. g. complications in polytraumatised patients, is a specific problem for which a high need exists in intensive care medicine.

Biomarkers for sepsis and resulting mortality can be detected by assaying blood samples. Changes in the concentration of the biomarkers can be used to indicate sepsis, risk of sepsis, progression of sepsis, remission from sepsis, and risk of mortality. Changes can be evaluated relative to datasets, natural or synthetic or semisynthetic control samples, or patient samples collected at different time points. Some biomarkers’ concentrations are elevated during disease and some are depressed. These are termed informative biomarkers. Some biomarkers are diagnostic in combination with others. Individual biomarkers may be weighted when used in combinations. Biomarkers can be assessed individually, isolated or in assays, in parallel assays, or in single-pot assays. See the ‘982 patent.

The early prediction or diagnosis of sepsis allows for clinical intervention before the disease rapidly progresses beyond initial stages to the more severe stages, such as severe sepsis or septic shock, which are associated with high mortality. Prediction or diagnosis has been accomplished, see the ‘573 patent, using a molecular diagnostics approach, involving comparing an individual’s profile of biomarker expression to profiles obtained from one or more control, or reference, populations, which may include a population who develops sepsis. Recognition of features in the individual’s biomarker profile that are characteristic of the onset of sepsis allows a clinician to diagnose the onset of sepsis from a bodily fluid isolated from the individual at a single point in time. The necessity of monitoring the patient over a period of time may be avoided, allowing clinical intervention before the onset of serious symptoms. Further, because the biomarker expression is assayed for its profile, identification of the particular biomarkers is unnecessary. The comparison of an individual’s biomarker profile to biomarker profiles of appropriate reference populations likewise can be used to diagnose SIRS in the individual. See the ‘573 patent.

Additional biomarkers for the diagnosis of sepsis include detection of inducible nitric oxide (NO) synthase (the enzyme responsible for overproduction of NO in inflammation), detection of endotoxin neutralization, and patterns of blood proteins. A panel of blood biomarkers for assessing a sepsis condition utilizes an iNOS indicator in combination with one or more indicators of patient predisposition to becoming septic, the existence of organ damage, or the worsening or recovering from a sepsis episode. See the ‘968 publication. Endotoxin neutralization as a biomarker for sepsis has been demonstrated, see the ‘530 publication, using methods specifically developed for detecting the neutralization in a human subject. This system has also provided methods for determining the effectiveness of a therapeutic agent for treating sepsis. See the ‘530 publication. Application of modern approaches of global proteomic has been used for the identification and detection of biological fluid biomarkers of neonatal sepsis. See the ‘652 publication. Methods using expression levels of the biomarkers Triggering Receptor Expressed on Myeloid cells-1 (TREM 1) and TREM-like receptor transcript-1 (TLT1) as an indication of the condition of the patient, alone or in combination with further sepsis markers have been used for the diagnosis, prognosis and prediction of sepsis in a subject. See the ‘370 patent. When levels of the biomarkers indicate the presence of sepsis, treatment of the patient with an antibiotic and/or fluid resuscitation treatment is indicated. See the ‘370 patent.

A multibiomarker-based outcome risk stratification model has been developed for adult septic shock. See the ‘869 publication. The approach employs methods for identifying, validating, and measuring clinically relevant, quantifiable biomarkers of diagnostic and therapeutic responses for blood, vascular, cardiac, and respiratory tract dysfunction, particularly as those responses relate to septic shock in adult patients. The model consists of identifying one or more biomarkers associated with septic shock in adult patients, obtaining a sample from an adult patient having at least one indication of septic shock, then quantifying from the sample an amount of one or more biomarkers, wherein the level of the biomarker(s) correlates with a predicted outcome. See the ‘869 publication.

The biomarker approach has also been used for prognostic purposes, by quantifying levels of metabolite(s) that predict severity of sepsis. See the ‘969 publication. The method involves measuring the age, mean arterial pressure, hematocrit, patient temperature, and the concentration of one or more metabolites that are predictive of sepsis severity. Analysis of a blood sample from a patient with sepsis establishes the concentration of the metabolite, after which the severity of sepsis infection can be determined by analyzing the measured values in a weighted logistic regression equation. See the ‘969 publication.

A method based on determination of blood levels of antitrypsin (ATT) or fragments thereof, and transthyretin (TTR) or fragments thereof has been described for the diagnosis, prediction or risk stratifcation for mortality and/or disease outcome of a subject that has or is suspected to have sepsis. See the ‘631 publication. Presence and/or level of ATT or its fragments is correlated with increased risk of mortality and/or poor disease outcome if the level of ATT is below a certain cut-off value and/or the level of fragments thereof is above a certain cut-off value. Similarly, increased risk of mortality and/or poor disease outcome exist if the level of TTR is below a certain cut-off value and/or the level of its fragments is also below a certain cut-off value. See the ‘631 publication.

### Clinical Data Analytics

The amount of data acquired electronically from patients undergoing intensive care has grown significantly during the past decade. Before it becomes knowledge for diagnostic and/or therapeutic purposes, bedside data must be extracted and organized to become information, and then an expert can interpret this information. Artificial intelligence applications in the intensive care unit represent an important use of such technologies. See Hanson et al. (2001). The use of computers to extract information from data and enhance analysis by the human clinical expert is a largely unrealized role for artificial intelligence. However, a variety of novel, computer-based analytic techniques have been developed recently. Although some of the earliest artificial intelligence applications were medically oriented, AI has not been widely accepted in medicine. Despite this, patient demographic, clinical, and billing data are increasingly available in an electronic format and therefore susceptible to analysis by intelligent software. The intensive care environment is therefore particularly suited to the implementation of AI tools because of the wealth of available data and the inherent opportunities for increased efficiency in inpatient care. A variety of new AI tools have become available in recent years that can function as intelligent assistants to clinicians, constantly monitoring electronic data streams for important trends, or adjusting the settings of bedside devices. The integration of these tools into the intensive care unit can be expected to reduce costs and improve patient outcomes. See Hanson et al. (2001).

Extensive efforts are being devoted to adding intelligence to medical devices, with various degrees of success. See Begley et al. (2000). Numerous technologies are currently used to create expert systems. Examples include: rule-based systems; statistical probability systems, Bayesian belief networks; neural networks; data mining; intelligent agents, multiple-agent systems; genetic algorithms; and fuzzy logic. Examples of specific uses include: pregnancy and child-care health information; pattern recognition in epidemiology, radiology, cancer diagnosis and myocardial infarction; discovery of patterns in treatments and outcomes in studies on epidemiology, toxicology and diagnosis; searches for and retrieval of relevant information from the internet or other knowledge repositories; and procedures that mimic evolution and natural selection to solve a problem.

In the modern healthcare system, rapidly expanding costs/complexity, the growing myriad of treatment options, and exploding information streams that often do not effectively reach the front lines hinder the ability to choose optimal treatment decisions over time. A general purpose (non-disease-specific) computational/artificial intelligence (AI) framework to address these challenges has been developed. See Bennett et al. (2013). This framework serves two potential functions, viz., a simulation environment for exploring various healthcare policies, payment methodologies, and providing the basis for clinical artificial intelligence. The approach combines Markov decision processes and dynamic decision networks to learn from clinical data and develop complex plans via simulation of alternative sequential decision paths while capturing the sometimes conflicting, sometimes synergistic interactions of various components in the healthcare system. It can operate in partially observable environments (in the case of missing observations or data) by maintaining belief states about patient health status and functions as an online agent that plans and re-plans as actions are performed and new observations are obtained.

Bennett and Hauser evaluated the framework using real patient data from an electronic health record, optimizing “clinical utility” in terms of cost-effectiveness of treatment (utilizing both outcomes and costs) and reflecting realistic clinical decision-making. The results of computational approaches were compared to existing treatment-as-usual (TAU) approaches, and the results demonstrate the feasibility of this approach. The AI framework easily outperformed the current TAU case-rate/fee-for-service models of healthcare. Using Markov decision processes, for instance, the cost per unit of outcome change (CPUC) was $189 vs. $497 for TAU (where lower CPUC is considered optimal) - while at the same time the AI approach could obtain a 30-35% increase in patient outcomes. According to Bennett and Hauser, modifying certain AI model parameters could further enhance this advantage, obtaining approximately 50% more improvement (outcome change) for roughly half the costs. Thus, given careful design and problem formulation, an AI simulation framework can approximate optimal decisions even in complex and uncertain environments.

### Artificial Intelligence for Sepsis Diagnosis

Development and assessment of a data-driven method that infers the probability distribution of the current state of patients with sepsis, likely trajectories, optimal actions related to antibiotic administration, prediction of mortality and length-of-stay have been conducted. See Tsoukalas et al. (2015). A data-driven, probabilistic framework for clinical decision support in sepsis-related cases was constructed, first defining states, actions, observations and rewards based on clinical practice, expert knowledge and data representations in an EHR dataset of 1492 patients. Partially Observable Markov Decision Process (POMDP) model was used to derive the optimal policy based on individual patient trajectories and the performance of the model-derived policies was evaluated in a separate test set. Policy decisions were focused on the type of antibiotic combinations to administer. Multi-class and discriminative classifiers were used to predict mortality and length of stay. Data-derived antibiotic administration policies led to a favorable patient outcome in 49% of the cases, versus 37% when the alternative policies were followed (P=1.3e-13).

Sensitivity analysis on the model parameters and missing data argued for a highly robust decision support tool that withstands parameter variation and data uncertainty. When the optimal policy was followed, 387 patients (25.9%) had 90% of their transitions to better states and 503 patients (33.7%) patients had 90% of their transitions to worse states (P=4.0e-06), while in the non-policy cases, these numbers are 192 (12.9%) and 764 (51.2%) patients (P=4.6e-117), respectively. Furthermore, the percentage of transitions within a trajectory that led to a better or better/same state were significantly higher by following the policy than for non-policy cases (605 vs 344 patients, P=8.6e-25). Mortality was predicted with an AUC of 0.7 and 0.82 accuracy in the general case and similar performance was obtained for the inference of the length-of-stay (AUC of 0.69 to 0.73 with accuracies from 0.69 to 0.82). Thus, a data-driven model was able to suggest favorable actions, predict mortality and length of stay as above. See Tsoukalas et al. (2015).

For sepsis monitoring and control, a computer-implemented alerting method has been developed. See the ‘449 patent. The method involves automatically extracting with a computer system, from records maintained for a patient under care in a healthcare facility, information from an electronic medical record, and obtaining with the computer system information about real-time status of the patient. The method also involves using the information from the electronic medical record and the information about the real-time status to determine whether the patient is likely to be suffering from dangerous probability of sepsis, using information from the electronic medical record to determine whether treatment for sepsis is already being provided to the patient, and electronically alerting a caregiver over a network if it is determined that a potentially dangerous level of sepsis exists and that treatment for sepsis is not already being provided. See the ‘449 patent.

The complexity of contemporary medical practice has impelled the development of different decision-support aids based on artificial intelligence and neural networks. Distributed associative memories are neural network models that fit well with the concept of cognition emerging from current neurosciences. A context-dependent autoassociative memory model has been reported, see Pomi et al. (2006), in which sets of diseases and symptoms are mapped onto bases of orthogonal vectors. A matrix memory stores associations between the signs and symptoms, and their corresponding diseases. In an implementation of the application with real data, a memory was trained with published data of neonates with suspected late-onset sepsis in a neonatal intensive care unit. A set of personal clinical observations was used as a test set to evaluate the capacity of the model to discriminate between septic and non-septic neonates on the basis of clinical and laboratory findings.

Results showed that matrix memory models with associations modulated by context could perform automated medical diagnoses. The sequential availability of new information over time makes the system progress in a narrowing process that reduces the range of diagnostic possibilities. At each step the system provides a probabilistic map of the different possible diagnoses to that moment. The system can incorporate the clinical experience, building in that way a representative database of historical data that captures geo-demographical differences between patient populations. The trained model succeeded in diagnosing late-onset sepsis within the test set of infants in the NICU: sensitivity 100%; specificity 80%; percentage of true positives 91%; percentage of true negatives 100%; accuracy (true positives plus true negatives over the totality of patients) 93.3%; and Cohen’s kappa index 0.84.

An electronic sepsis surveillance system (ESSV) was developed to identify severe sepsis and determine its time of onset. ESSV sensitivity and specificity were evaluated during an 11-day prospective pilot study and a 30-day retrospective trial. See Brandt et al. (2015), ESSV diagnostic alerts were compared with care team diagnoses and with administrative records, using expert adjudication as the standard for comparison. ESSV was 100% sensitive for detecting severe sepsis but only 62.0% specific. During the pilot study, the software identified 477 patients, compared with 18 by adjudication. In the 30-day trial, adjudication identified 164 severe sepsis patients, whereas ESSV detected 996. ESSV was more sensitive but less specific than care team or administrative data. ESSV-identified time of severe sepsis onset was a median of 0 hours later than by adjudication (interquartile range = 0.05).

A retrospective, data-driven analysis, based on neural networks and rule-based systems has been applied to the data of two clinical studies of septic shock diagnosis. See Brause et al. (2001). The approach included steps of data mining, i.e., building up a database, cleaning and preprocessing the data and finally choosing an adequate analysis for the patient data. Two architectures based on supervised neural networks were chosen. Patient data was classified into two classes (survived and deceased) by a diagnosis based either on the black-box approach of a growing RBF network, and otherwise on a second network which could be used to explain its diagnosis by human understandable diagnostic rules. Advantages and drawbacks of these classification methods for an early warning system were identified.

It has been recommended that mortality risk stratification or severity-of-illness scoring systems be utilized in clinical trials and in practice to improve the precision of evaluation of new therapies for the treatment of sepsis, to monitor their utilization and to refine their indications. See Barriere et al. (1995). With the increasing influence of managed care on healthcare delivery, there will be increased demand for techniques to stratify patients for cost-effective allocation of care. Severity of illness scoring systems are widely utilized for patient stratification in the management of cancer and heart disease.

Mortality risk prediction in sepsis has evolved from identification of risk factors and simple counts of failing organs, to techniques that mathematically transform a raw score, comprised of physiologic and/or clinical data, into a predicted risk of death. Most of the developed systems are based on global ICU populations rather than upon sepsis patient databases. A few systems are derived from such databases. Mortality prediction has also been carried out from assessments of plasma concentrations of endotoxin or cytokine (IL-1, IL-6, TNF-α). While increased levels of these substances have been correlated with increased mortality, difficulties with bioassay and their sporadic appearance in the bloodstream prevent these measurements from being practically applied. The calibration of risk prediction methods comparing predicted with actual mortality across the breadth of risk for a population of patients is excellent, but overall accuracy in individual patient predictions is such that clinical judgment must remain a major part of decision-making. With databases of appropriate patient information increasing in size and complexity, clinical decision making requires the innovation of a reliable scoring system. See Angus et al. (2013).

Dynamic Bayesian Networks, a temporal probabilistic technique to model a system whose state changes over time, was used to detect the presence of sepsis soon after the patient visits the emergency department. See Nachimuthu et al. (2012). A model was built, trained and tested using data of 3,100 patients admitted to the emergency department, and the accuracy of detecting sepsis using data collected within the first 3 hours, 6 hours, 12 hours and 24 hours after admission was determined. The area under the curve was 0.911, 0.915, 0.937 and 0.944 respectively.

Application of new knowledge based methods to a septic shock patient database has been proposed, and an approach has been developed that uses wrapper methods (bottom-up tree search or ant feature selection) to reduce the number of properties. See Fialho et al. (2012). The goal was to estimate, as accurately as possible, the outcome (survived or deceased) of septic shock patients. A wrapper feature selection based on soft computing methods was applied to a publicly available ICU database. Fuzzy and neural models were derived and features were selected using a tree search method and ant feature selection.

An attempt has been made to support medical decision making using machine learning for early detection of late-onset neonatal sepsis from off-the-shelf medical data and electronic medical records (EMR). See Mani et al. (2014). Data used were from 299 infants admitted to the neonatal intensive care unit and evaluated for late-onset sepsis. Gold standard diagnostic labels (sepsis negative, culture positive sepsis, culture negative/clinical sepsis) were assigned based on all the laboratory, clinical and microbiology data available in EMR. Only data that were available up to 12 h after phlebotomy for blood culture testing were used to build predictive models using machine learning (ML) algorithms. Sensitivity, specificity, positive predictive value and negative predictive value of sepsis treatment of physicians were compared with predictions of models generated by ML algorithms.

Treatment sensitivity of all the nine ML algorithms and specificity of eight out of the nine ML algorithms tested exceeded that of the physician when culture-negative sepsis was included. When culture negative sepsis was excluded both sensitivity and specificity exceeded that of the physician for all the ML algorithms. The top three predictive variables were the hematocrit or packed cell volume, chorioamnionitis and respiratory rate. See Rangel-Fausto et al. (1995); and Mani et al. (2014).

The importance of preprocessing in clinical databases has been recognized. Specifically in intensive care units, data is often irregularly recorded, contain a large amount of missing values and sampling times are uneven. A systematic preprocessing procedure has been proposed, see Marques et al. (2011), that can be generalized to common clinical databases. This procedure was applied to a known septic shock patient database and classification results were compared with previous studies. The goal was to estimate, as accurately as possible, the outcome (survived or deceased) of these septic shock patients. Neural modeling was used for classification, and results showed that preprocessing improved classifier accuracy. See Marques et al. (2011).

### SUMMARY OF THE INVENTION

The present invention relates to the composition and use of clinical parameters (or features) for the prediction or risk stratification for Systemic Inflammatory Response Syndrome (SIRS) several hours to days before SIRS symptoms are observable for a definitive diagnosis in a patient, and relates to the development of groups of parameters and corresponding prediction models for predicting onset of a disease, e.g., as SIRS. The ability to predict the onset of SIRS, prior to the appearance of clinical symptoms, enables physicians to initiate therapy in an expeditious manner, thereby improving outcomes. This applies to patients that have non-infectious SIRS or patients with SIRS that progress to sepsis. The ability to predict a disease, e.g., SIRS, is useful for healthcare professionals to provide early prophylactic treatment for hospitalized patients, who will otherwise develop sepsis and/or conditions — such as pancreatitis, trauma, or burns — that share symptoms identical or similar to, for example, SIRS.

Moreover, such a predictive ability can also be applied to enhance patient care during clinical trials. A clinical trial is a prospective biomedical or behavioral research studies on human subjects that is designed to answer specific questions about biomedical or behavioral interventions (novel vaccines, drugs, treatments, devices or new ways of using known interventions), generating safety and efficacy data. The patients can include patients who develop SIRS or SIRS-like symptoms when they are enrolled in clinical trials investigating a variety of pre-existing conditions. For example, a medical device company could be conducting a trial for an implantable device such as hip replacement system, or a pharmaceutical company could be conducting a trial for a new immunosuppressant for organ recipients. In both scenarios, the clinical trial protocol would concentrate on functional and recovery measurements. If trial investigators had access to a method that predicted which patients were infected during the operation, or at any time during the trial, they would be able to provide early treatment, and minimize adverse events and patient dropout. Correspondingly the same method can also be used to screen patients during the initial phase of patient enrollment: a potential enrollee predicted to develop SIRS could first be treated or excluded from the trial, thereby reducing adverse or confounding results during the trial.

The invention is based on combinatorial extraction and iterative prioritization of clinical parameters and measurements (or, collectively, “features”) commonly available in healthcare settings in the form of common patient measurements, laboratory tests, medications taken, fluids and solids entering and leaving the patient by specified routes, to correlate their presence and temporal fluctuations to whether a patient would ultimately develop SIRS. This group of clinical parameter combinations has not been previously associated with SIRS or related to its progression and risk stratification. The invention relates, in general, to the identification and prioritization of these clinical parameters and measurements, or combinations thereof, for the prediction (or predictive modeling) of SIRS. As shown in the below timeline, the invention enables the prediction of SIRS well prior to a prediction time (and/or a time of diagnosis) enabled by existing technologies.

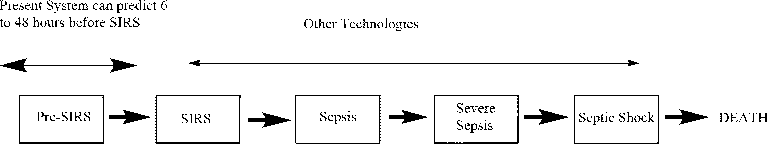

### BRIEF DESCRIPTION OF FIGURES

FIG. 1 illustrates an embodiment of the system utilized in the present disclosure.

### DETAILED DESCRIPTION

This invention describes the identification of seemingly unrelated physiologic features and clinical procedures, combinations of which can be used to predict accurately the likelihood of a SIRS-negative patient becoming diagnosed as SIRS-positive 6 to 48 hours (e.g., 6, 12, 24 or 48 hours) later.

The MIMIC II database contains a variety of hospital data for four intensive care units (ICUs) from a single hospital, the Beth Israel Deaconess Medical Center (BIDMC) in Boston. MIMIC itself stands for “Multiparameter Intelligent Monitoring in Intensive Care,” and this second version is an improvement on the original installment. The hospital data tabulated is time-stamped and contains physiological signals and measurements, vital signs, and a comprehensive set of clinical data representing such quantitative data as medications taken (amounts, times, and routes); laboratory tests, measurements, and outcomes; feeding and ventilation regimens, diagnostic assessments, and billing codes representing services received. MIMIC II contains information for over 33,000 patients collected between 2001 and 2008 from the medical ICU (MICU), surgical ICU (SICU), coronary care unit (CCU) and cardiac surgery recovery unit (CSRU), as well as the neonatal ICU (NICU). Operationally MIMIC II is organized as a relational PostgreSQL database that can be queried using the SQL language, for convenience and flexibility. The database is organized according to individual patients, each denoted by a unique integer identification number. A particular patient may have experienced multiple hospital admissions and multiple ICU stays for each admission, which are all accounted for in the database. To comply with the Health Insurance Portability and Accountability Act (HIPAA), the individuals in the database were de-identified by removing protected health information (PHI). Moreover, the entire time course for each patient (e.g., birthday, all hospital admissions, and ICU stays) was time-shifted to a hypothetical period in the future, to further reduce the possibility of patient re-identification.

### Data Preparation

Although the MIMIC II database was used as a source of measurements and other data for the invention, the invention disclosed here is not limited by the MIMIC II database or the specific measurements, representations, scales, or units from the BIDMC or the MIMIC II database. For example, the units that are used to measure a feature for use in the invention may vary according to the lab or location where the measurement occurs. The standard dose of medication or route of administration may vary between hospitals or hospital systems, or even the particular member of a class of similar medications that are prescribed for a given condition may vary. Mapping of the specific features found in the MIMIC II database to those used in another hospital system are incorporated into the invention disclosed here to make use of this invention in a different hospital. For example, if the MIMIC II database measures the weight of patients in pounds and another hospital does so in kilograms, one of ordinary skill in the art would appreciate that it is a simple matter to convert the patients’ weights from kilograms to pounds. Likewise, it is straightforward to adjust the predictive formula of the invention to accept kilograms instead of pounds. This sort of mapping between features also can be done between medications that carry out the same functions, but may differ in standard dosages, and/or alternative laboratory measurements that measure the same parameter, vital sign or other aspect in a patient, etc.

In addition, rather than mapping feature-to-feature as described in the above paragraph and then using the exemplary models presented here with the newly mapped features, it is straightforward to use the methods of the invention taught here to take existing hospital datasets and retrain models in accordance with the techniques of the invention described herein. Those models can then be used predictively, in the manner of the invention shown here. The same feature removal and feature selection methods can be used, or the features found useful here can guide hand-curated feature selection methods. All of this would be apparent to one of ordinary skill in the art.

The MIMIC II Database is available online at the following site [https://physionet.org/mimic2/], and is incorporated herein by reference in its entirety. As a person of ordinary skill in the art would appreciate, the MIMIC II database can be readily and easily accessed as follows. Information at the website https://physionet.org/mimic2/mimic2_access.shtml describes how to access the MIMIC II clinical database. First one needs to create a PhysioNetWorks account at https://physionet.org/pnw/login. One then follows the directions at https://physionet.org/works/MIMICIIClinicalDatabase/access.shtml, which includes completing a training program in protecting human research participants (which can be accomplished online) because of research rules governing human subjects data. Finally, one applies for access to the database by filling out an application, including certification from the human subjects training program and a signed data use agreement. These are common steps familiar to one of ordinary skill in the art when dealing with such medical data on human subjects and one of ordinary skill in the art would expect such steps to be taken. Approved applicants, such as a person of ordinary skill in the art, receive instructions by email for accessing the database. When updated (including the recent release of the MIMIC III database), the updated features can be used as described herein for prediction of SIRS, and are within the scope of the invention.

Data were obtained from the MIMIC II Database from the tables representing chart measurements, laboratory measurements, drugs, fluids, microbiology, and cumulative fluids for patients. See Saeed et al. (2011). The following tables were used to extract patient data used according to the invention for prediction:

The chart events table contains charted data for all patients. We recorded the patient id, the item id, the time stamp, and numerical values.

The lab events table contains laboratory data for all patients. We recorded the patient id, the item id, the time stamp, and numerical values.

The io events table contains input and output (fluid transfer) events for all patients. We recorded the patient id, the item id, the time stamp, and numerical value (generally of the fluid volume).

The micro events table contains microbiology data for all patients. We recorded the patient id, the item id, the time stamp, and the result interpretation. The result interpretation that we gather is based on 2 categories ‘R’ (resistant) and ‘S’ (sensitive) that are mapped to 1 and -1 values, respectively.

The med events table contains medication data for all patients. We recorded the patient id, the item id, the time stamp, and the medication dose.

The total balance (totalbal) events table contains the total balance of input and output events. We recorded the patient id, the item id, the time stamp, and the cumulative io volume.

As a person of ordinary skill in the art would appreciate, the above entries, those in Tables 1 to 7 herein, and those in the MIMIC II database correspond to features (as shown in the MIMIC II database and below) identified by well-known abbreviations that have well-known meanings to those of ordinary skill in the art. Moreover, the corresponding entries, such as measurements and other parameters, in the MICMI II database are features in accordance with the invention.

All patients with sufficient data in the MIMIC II database, except those that spent any time in the neonatal ICU, were included in the development. Patients who met at least two of the four conditions for SIRS simultaneously at some point in their stay were identified from the database. (The four conditions are a temperature of less than 36° C. or greater than 38° C., a heart rate of greater than 90 beats per minute, a respiratory rate of greater than 20 breaths per minute, and a white blood cell (WBC) count of less than 4,000 per microliter or greater than 12,000 per microliter.) We checked for the occurrence of SIRS using all 6 possible 2-condition cases for each patient during their ICU stays without repetition at any given time using time-stamped chart times. The occurrence of SIRS is modeled as a point process which requires that the 2 or more SIRS conditions occur simultaneously. Heart rate was extracted from item id 211 in the chart events table. Respiration rate measurement was extracted by item ids 219, 615, and 618 in the chart events table. Temperatures were extracted from item ids 676, 677, 678, and 679 in the chart events table. Finally, WBC measurements were extracted from item ids 50316 and 50468 in the lab events table. Where multiple sources of a measurement were available, the one most recently updated at the time point was used.

Each time SIRS conditions occurred in a patient, we recorded the time stamped date and time of the SIRS occurrence and the patient id. We used the timestamps of positive patients to collect data from the 7 tables as described above 6, 12, 24, and 48 hours (the “time point”) before the occurrence of SIRS, using the most recent data nearest the time point for each patient, but no later than 1 week before the onset or any time before the current stay. For all patients for which no SIRS occurrence was found (SIRS negative patients), we recorded their ids. Using their ids, we collected data for 6, 12, 24 and 48 hours before some point in their last recorded stay. The ids for positive patients and negative patients are disjoint sets. The numbers of positive, negative, and total patients for the 48-hour time point was 9,029, 5,249, and 14,278, respectively; for the 24-hour time point 11,024, 5,249, and 16,273; for the 12-hour time point 13,033, 5,249, and 18,282; and for the 6-hour time point 15,075, 5,249, and 20,324. These numbers are different at different time points (and grow for shorter times) because fewer patients were present in the ICU 48 hours before the onset of SIRS than were present 6 hours before the onset of SIRS.

Data were normalized to a mean of zero and standard deviation of one. That is, a normalized version of each datum was created by subtracting the mean for each feature (taken across all occurrences for each feature or measurement type) and divided by the standard deviation (taken across the same distribution). The distribution of each feature property in the data was compared between the positives (patients who met the criteria for SIRS) and negatives (those that did not) at each of the four time points using the Bhattacharyya distance. That is, a histogram giving the population of SIRS-positive patients as a function of the measured value of some feature was compared to the same histogram but for SIRS-negative patients, and the Bhattacharyya distance was computed between these two histogram distributions. Any feature whose Bhattacharyya distance was less than 0.01 at all four time points was removed from further consideration. See Bhattacharyya (1943). The list of features after this step and used in the next steps of the analysis, as well as the mean and standard deviation of each feature, is shown in Table 1.

TABLE 1

The list of features extracted from the MIMIC II database after the Bhattacharyya procedure described in the text is given in the first column (using the MIMIC II identifiers or IDs), the mean for each feature across the patient measurements used in this study are given in the second column, and the standard deviation for each feature across the same distribution of patient measurements is given in the third column

| Feature ID | Mean | Standard Deviation |
|---|---|---|
| chart 2 | 0.00085647 | 0.02855790 |
| chart 4 | 0.09469113 | 3.17506964 |
| chart 5 | 0.09931363 | 3.34506569 |
| chart 25 | 0.36479470 | 7.86772704 |
| chart 26 | 0.52791918 | 12.94497598 |
| chart 29 | -0.05025127 | 1.71765410 |
| chart 63 | 0.01411262 | 0.44388419 |
| chart 65 | 0.25089882 | 11.57072921 |
| chart 79 | 0.12028499 | 3.70876085 |
| chart 92 | 0.30364889 | 4.83353618 |
| chart 142 | -0.01564594 | 3.97578730 |
| chart 146 | 0.60699444 | 20.67946954 |
| chart 181 | 0.22106800 | 5.34978160 |
| chart 186 | 0.58731250 | 5.66550401 |
| chart 192 | 0.15565999 | 4.92768741 |
| chart 221 | 0.04279908 | 0.53402450 |
| chart 226 | 0.05647857 | 1.15417713 |
| chart 440 | 0.02739493 | 0.39652687 |
| chart 441 | 0.00281059 | 0.10200107 |
| chart 442 | 0.89347991 | 10.75176971 |
| chart 449 | 0.13689356 | 1.32076222 |
| chart 472 | 15.32213520 | 114.07135964 |
| chart 473 | 7.60184896 | 55.95736185 |
| chart 481 | 0.27143391 | 4.52794496 |
| chart 482 | 0.24527245 | 5.40135244 |
| chart 483 | 0.16893122 | 3.74289763 |
| chart 484 | 0.43437456 | 7.34509454 |
| chart 485 | 0.23984452 | 4.16349239 |
| chart 490 | 1.00636153 | 20.52072585 |
| chart 491 | 0.62665725 | 4.21715369 |
| chart 492 | 1.82722530 | 8.25509216 |
| chart 494 | 0.00080165 | 0.03223063 |
| chart 496 | 0.01173124 | 0.43029790 |
| chart 498 | 0.10166860 | 0.81219774 |
| chart 503 | 0.01031891 | 0.62436918 |
| chart 512 | 0.96757982 | 16.61775297 |
| chart 517 | 2.84696589 | 13.68963861 |
| chart 595 | 1.66920135 | 10.23611224 |
| chart 601 | 0.41964772 | 4.03303813 |
| chart 602 | 0.20118514 | 1.91755499 |
| chart 607 | 0.01587524 | 0.86210144 |
| chart 624 | 0.09279608 | 2.99007718 |
| chart 626 | 42.64198104 | 215.05044423 |
| chart 664 | 1.15885278 | 9.03981262 |
| chart 670 | 0.02115207 | 0.76604451 |
| chart 671 | 0.00072793 | 0.02546059 |
| chart 682 | 63.59648566 | 189.80611025 |
| chart 683 | 62.40508426 | 186.76148116 |
| chart 686 | 0.01310848 | 0.34099410 |
| chart 725 | 0.00427231 | 0.23324051 |
| chart 727 | 0.01532077 | 0.85030301 |
| chart 773 | 13.59515581 | 58.13663911 |
| chart 779 | 26.86192608 | 68.96123873 |
| chart 781 | 9.25061379 | 16.73587842 |
| chart 784 | 84.80022284 | 458.89082645 |
| chart 785 | 5.12691634 | 31.66196336 |
| chart 789 | 7.21857636 | 35.08988939 |
| chart 792 | 0.27696501 | 8.44017613 |
| chart 793 | 15.37957230 | 265.41501495 |
| chart 807 | 27.76192457 | 59.15571578 |
| chart 809 | 0.00328444 | 0.09765775 |
| chart 811 | 53.96478158 | 72.76417086 |
| chart 815 | 0.44755771 | 0.89990497 |
| chart 817 | 29.91306924 | 253.35321509 |
| chart 818 | 0.23468293 | 1.49861009 |
| chart 821 | 0.74681259 | 1.04674692 |
| chart 826 | 0.01572763 | 0.70942705 |
| chart 828 | 83.80563920 | 118.11615467 |
| chart 835 | 0.21953355 | 3.85647957 |
| chart 836 | 4.99966513 | 38.32880772 |
| chart 844 | 0.02802563 | 1.51344740 |
| chart 850 | 6.46337022 | 36.52996134 |
| chart 851 | 0.10410908 | 1.51648228 |
| chart 856 | 0.03264113 | 0.81953706 |
| chart 1162 | 7.12888622 | 15.20614694 |
| chart 1223 | 0.00487930 | 0.29281632 |
| chart 1340 | 0.00917443 | 0.37187792 |
| chart 1390 | 13.26147798 | 105.84980386 |
| chart 1391 | 8.10418715 | 58.66008183 |
| chart 1397 | 1.60976889 | 9.98859281 |
| chart 1401 | 13.30554577 | 105.97517485 |
| chart 1402 | 6.59470944 | 52.04974798 |
| chart 1411 | 0.03272241 | 1.05123619 |
| chart 1486 | 0.01720523 | 0.66680868 |
| chart 1520 | 2.30231283 | 19.78048269 |
| chart 1524 | 5.55297194 | 30.98367694 |
| chart 1526 | 15.37957230 | 265.41501495 |
| chart 1528 | 12.79354842 | 69.03612583 |
| chart 1529 | 41.13133466 | 67.21985389 |
| chart 1531 | 0.20717948 | 1.46503589 |
| chart 1532 | 0.58986881 | 0.99893334 |
| chart 1537 | 0.02334010 | 1.40616562 |
| chart 1540 | 5.08306486 | 33.01429097 |
| chart 1546 | 0.01264883 | 0.42526786 |
| chart 1565 | 0.00798070 | 0.37051630 |
| chart 1624 | 0.16967560 | 3.58526348 |
| chart 1671 | 0.04965681 | 2.65454906 |
| chart 2139 | 0.07565662 | 3.23055891 |
| chart 5683 | 0.09686892 | 4.15236669 |
| chart 5816 | 0.00266038 | 0.11524787 |
| chart 5818 | 0.14924620 | 2.05413428 |
| chart 6702 | 0.24175311 | 4.39982998 |
| chart 6711 | 0.00245132 | 0.14175629 |
| chart 6712 | 0.00294159 | 0.15337553 |
| lab 50001 | 23.22076458 | 100.41719985 |
| lab 50013 | 7.69303340 | 22.24716472 |
| lab 50017 | 0.32463216 | 2.03215748 |
| lab 50019 | 50.42589412 | 87.47914829 |
| lab 50030 | 0.24833007 | 0.49748206 |
| lab 50038 | 5.03150301 | 377.69248727 |
| lab 50042 | 0.01275856 | 0.54636087 |
| lab 50044 | 6.24396591 | 567.17187703 |
| lab 50055 | 0.05298472 | 1.03827871 |
| lab 50056 | 0.15714736 | 3.52138287 |
| lab 50059 | 297.50420651 | 13326.87658960 |
| lab 50061 | 70.64888293 | 113.60306928 |
| lab 50062 | 31.87699226 | 170.59063119 |
| lab 50064 | 1.24808645 | 10.19100689 |
| lab 50071 | 0.12456226 | 8.81696541 |
| lab 50072 | 0.02155964 | 0.55970162 |
| lab 50073 | 36.01986023 | 200.55947412 |
| lab 50075 | 1.21495658 | 12.11099863 |
| lab 50076 | 0.29621912 | 3.03200984 |
| lab 50077 | 6.36559620 | 241.37014245 |
| lab 50078 | 1.23008325 | 72.98495642 |
| lab 50082 | 1.74522064 | 51.65243614 |
| lab 50086 | 117.89271088 | 464.52399231 |
| lab 50087 | 5.49516077 | 25.82523777 |
| lab 50089 | 1.00971800 | 6.69502589 |
| lab 50093 | 1.97097081 | 27.92531610 |
| lab 50094 | 0.12298641 | 5.70363476 |
| lab 50098 | 0.05717421 | 3.36660155 |
| lab 50099 | 4.61501799 | 34.14694339 |
| lab 50101 | 50.01726542 | 193.59327847 |
| lab 50102 | 0.06362372 | 1.10232089 |
| lab 50106 | 0.13661110 | 3.26226671 |
| lab 50107 | 0.45142877 | 16.02191354 |
| lab 50109 | 6.10556506 | 69.46251026 |
| lab 50115 | 12.86690919 | 57.08667334 |
| lab 50120 | 20.83012709 | 1090.70498689 |
| lab 50129 | 7.27576402 | 89.66349837 |
| lab 50130 | 37.76052003 | 280.74132309 |
| lab 50138 | 17.53646157 | 131.58751595 |
| lab 50144 | 15.88692719 | 66.47903839 |
| lab 50146 | 0.04576709 | 1.07028846 |
| lab 50152 | 0.88600778 | 69.38537896 |
| lab 50154 | 5.49521705 | 49.55527991 |
| lab 50158 | 0.20605360 | 4.50194703 |
| lab 50164 | 0.02401253 | 0.44999333 |
| lab 50165 | 2.36349512 | 34.51166865 |
| lab 50167 | 2.17450584 | 231.32404185 |
| lab 50173 | 31.31925548 | 77.23835655 |
| lab 50179 | 0.39315773 | 5.09912488 |
| lab 50181 | 94.34078891 | 272.00067686 |
| lab 50190 | 40.72391222 | 100.42871234 |
| lab 50195 | 307.93628798 | 2596.86410786 |
| lab 50196 | 0.01733043 | 0.39757285 |
| lab 50202 | 0.20942270 | 5.09167981 |
| lab 50204 | 2.09826473 | 37.28375222 |
| lab 50208 | 0.07031797 | 2.95357221 |
| lab 50212 | 32.28998809 | 1822.22575476 |
| lab 50216 | 0.01408811 | 0.74006392 |
| lab 50217 | 0.27062614 | 5.69846437 |
| lab 50218 | 1.92383387 | 77.28189759 |
| lab 50225 | 0.00780922 | 0.76859856 |
| lab 50226 | 0.04006163 | 2.41405113 |
| lab 50232 | 0.04272307 | 3.01560050 |
| lab 50235 | 0.14869029 | 3.05652875 |
| lab 50237 | 0.09455106 | 2.75161652 |
| lab 50239 | 0.00166690 | 0.05087849 |
| lab 50240 | 1.51154457 | 14.25533386 |
| lab 50241 | 9.39977588 | 352.74628108 |
| lab 50247 | 0.09132932 | 5.45906917 |
| lab 50250 | 0.13962040 | 6.88993531 |
| lab 50251 | 0.01882266 | 0.97346587 |
| lab 50252 | 0.06709623 | 2.62018469 |
| lab 50253 | 0.13093571 | 5.12523144 |
| lab 50254 | 7.03257581 | 97.77120331 |
| lab 50255 | 4.68506799 | 173.98288638 |
| lab 50258 | 0.10672830 | 2.78573420 |
| lab 50259 | 4.74681328 | 155.39408808 |
| lab 50260 | 11.65093150 | 1385.01063408 |
| lab 50261 | 4.34458608 | 361.75381215 |
| lab 50263 | 1.98564808 | 13.44359622 |
| lab 50265 | 0.10524350 | 3.01526596 |
| lab 50266 | 0.05532988 | 6.45357037 |
| lab 50273 | 0.28632336 | 4.39336506 |
| lab 50276 | 0.08971186 | 1.67879510 |
| lab 50277 | 7.90530293 | 24.59478854 |
| lab 50278 | 6.32139909 | 74.56982431 |
| lab 50279 | 0.01213055 | 0.55230286 |
| lab 50284 | 0.07129500 | 1.75647837 |
| lab 50285 | 23.11024163 | 265.93045864 |
| lab 50287 | 9.76370056 | 179.33304781 |
| lab 50288 | 0.61341189 | 9.78191108 |
| lab 50302 | 0.00545595 | 0.25048838 |
| lab 50304 | 0.33916680 | 3.88564769 |
| lab 50313 | 191.71262224 | 7100.57705838 |
| lab 50314 | 45.76779934 | 2198.66177537 |
| lab 50317 | 10.82248262 | 127.26177844 |
| lab 50318 | 4.19766095 | 54.87326127 |
| lab 50319 | 7.08626305 | 87.10633523 |
| lab 50320 | 18.03138369 | 190.04396439 |
| lab 50322 | 0.05020661 | 1.35116710 |
| lab 50323 | 0.06843045 | 1.76870860 |
| lab 50330 | 0.38097072 | 6.10177124 |
| lab 50335 | 0.14723227 | 2.86477532 |
| lab 50356 | 0.30287054 | 3.16345475 |
| lab 50357 | 0.01400486 | 0.57214803 |
| lab 50367 | 0.51411003 | 5.28545582 |
| lab 50374 | 0.85250035 | 28.14780910 |
| lab 50378 | 44.27892180 | 127.19341944 |
| lab 50382 | 276.98644508 | 1654.09627098 |
| lab 50385 | 0.00039817 | 0.02110789 |
| lab 50390 | 0.00601975 | 0.34739895 |
| lab 50395 | 0.00910492 | 0.46236088 |
| lab 50404 | 0.12722370 | 5.45882054 |
| lab 50427 | 0.00478592 | 0.26923167 |
| lab 50428 | 231.69180006 | 112.32668925 |
| lab 50434 | 0.02742736 | 0.73329938 |
| lab 50436 | 0.47300042 | 7.79233392 |
| lab 50437 | 0.07886259 | 2.88194189 |
| lab 50438 | 0.12774898 | 3.39834450 |
| lab 50441 | 0.02315450 | 0.49641135 |
| lab 50451 | 3.21353796 | 14.71240938 |
| lab 50460 | 0.29951090 | 5.80080856 |
| lab 50461 | 0.04622496 | 2.05194982 |
| lab 50463 | 0.64392486 | 11.17676477 |
| lab 50465 | 0.13695896 | 4.38723417 |
| lab 50466 | 0.21007844 | 7.29724834 |
| lab 50469 | 0.02087127 | 1.31441149 |
| lab 50473 | 0.00754074 | 0.48035113 |
| lab 50510 | 0.00241630 | 0.13329013 |
| lab 50511 | 0.00172760 | 0.10773917 |
| lab 50513 | 0.00855629 | 0.47020900 |
| lab 50526 | 56.17471342 | 1721.05586913 |
| lab 50530 | 0.00098053 | 0.05020359 |
| lab 50537 | 0.06201849 | 1.59872762 |
| lab 50541 | 0.06614488 | 1.51504583 |
| lab 50545 | 0.34937783 | 5.41626284 |
| lab 50546 | 300.30688238 | 18625.78104190 |
| lab 50548 | 127.38286758 | 3309.11019170 |
| lab 50549 | 0.00105057 | 0.05486823 |
| lab 50550 | 0.00322174 | 0.26541980 |
| lab 50551 | 0.00119064 | 0.08156087 |
| lab 50560 | 0.01072839 | 0.41973561 |
| lab 50565 | 0.06933131 | 2.32126071 |
| lab 50567 | 1.21979911 | 81.36029950 |
| lab 50579 | 0.01884017 | 0.65461564 |
| lab 50587 | 0.20333847 | 3.18805892 |
| lab 50588 | 0.15634076 | 2.88158136 |
| lab 50589 | 0.02133352 | 0.77524117 |
| lab 50598 | 311.52946958 | 24113.47723120 |
| lab 50599 | 18.46205351 | 1326.02925765 |
| lab 50600 | 0.00266144 | 0.16482594 |
| lab 50603 | 0.03637297 | 1.22923047 |
| lab 50604 | 0.00632091 | 0.24963091 |
| lab 50609 | 0.14016552 | 1.90214299 |
| lab 50614 | 0.36558692 | 4.41471535 |
| lab 50616 | 298.68821497 | 6959.19944596 |
| lab 50617 | 35.65359878 | 1333.62922289 |
| lab 50632 | 0.00077042 | 0.04183721 |
| lab 50641 | 35.64576586 | 158.00230185 |
| lab 50647 | 3.57980310 | 17.36721303 |
| lab 50652 | 0.00539291 | 0.25281504 |
| lab 50655 | 15.82295535 | 61.95048456 |
| lab 50659 | 0.11790867 | 13.25911071 |
| lab 50664 | 0.01456787 | 0.50219925 |
| lab 50675 | 0.00616333 | 0.48115179 |
| lab 50687 | 0.01151422 | 0.69778365 |
| lab 50689 | 0.00814890 | 0.56889197 |
| lab 50690 | 0.00042618 | 0.02947423 |
| lab 50699 | 0.10274260 | 1.25007695 |
| io 48 | 1.73658776 | 44.28782162 |
| io 49 | 2.71781762 | 66.99138798 |
| io 51 | 0.34689732 | 13.08254103 |
| io 52 | 10.17957697 | 142.29269898 |
| io 53 | 1.69344446 | 67.30777999 |
| io 55 | 949.40834150 | 2722.10335736 |
| io 58 | 0.90888080 | 27.62156522 |
| io 59 | 6.91924639 | 60.88424044 |
| io 60 | 54.64588878 | 302.96267120 |
| io 61 | 22.99544754 | 204.39111258 |
| io 63 | 0.97247514 | 62.50643457 |
| io 64 | 26.86115002 | 397.70183709 |
| io 65 | 34.37260120 | 222.61955003 |
| io 66 | 1.19775179 | 72.37468407 |
| io 68 | 0.33267965 | 19.37697438 |
| io 70 | 0.75885978 | 22.67961529 |
| io 71 | 4.37424709 | 79.19413878 |
| io 72 | 1.20759210 | 49.73564850 |
| io 73 | 0.42639025 | 17.85125962 |
| io 74 | 0.36104496 | 12.30809779 |
| io 76 | 28.97170472 | 179.26026786 |
| io 77 | 0.54279311 | 21.93176386 |
| io 80 | 0.35992436 | 29.88552815 |
| io 84 | 0.18721109 | 10.40747286 |
| io 85 | 0.47275529 | 19.61861269 |
| io 87 | 0.48010926 | 30.50209458 |
| io 88 | 0.24324135 | 9.76926775 |
| io 91 | 0.73833870 | 25.44325971 |
| io 92 | 0.04706542 | 2.97974170 |
| io 93 | 0.37785404 | 21.59698881 |
| io 94 | 42.35425130 | 388.86403879 |
| io 97 | 0.52794509 | 31.04533368 |
| io 102 | 279.29773078 | 753.16871790 |
| io 104 | 195.77040202 | 638.74932511 |
| io 106 | 153.23973362 | 693.60841995 |
| io 107 | 14.33074544 | 207.92960485 |
| io 123 | 24.94261801 | 292.21717716 |
| io 124 | 208.90881076 | 954.80094392 |
| io 125 | 20.33225942 | 315.34376435 |
| io 128 | 1.44256899 | 39.17880469 |
| io 130 | 56.51977168 | 357.54608426 |
| io 131 | 28.55636877 | 277.83545815 |
| io 132 | 6.35137975 | 43.61830338 |
| io 133 | 8.38249132 | 74.27164202 |
| io 134 | 320.54597983 | 1127.87536431 |
| io 137 | 10.25941527 | 71.50864647 |
| io 138 | 0.04529696 | 2.90709517 |
| io 139 | 2.74161764 | 40.66924563 |
| io 140 | 3.69234248 | 66.42834639 |
| io 141 | 14.57634223 | 125.26022645 |
| io 142 | 14.53412826 | 215.49989625 |
| io 143 | 0.93220339 | 32.06141061 |
| io 144 | 61.25270229 | 296.11008219 |
| io 147 | 1.08450232 | 28.60256873 |
| io 149 | 0.93797075 | 20.55435165 |
| io 151 | 60.70526334 | 366.57431000 |
| io 152 | 30.57171289 | 275.77108826 |
| io 154 | 11.13338003 | 161.83482830 |
| io 155 | 11.39340244 | 121.46558083 |
| io 158 | 8.13713405 | 165.85419264 |
| io 159 | 1.93360415 | 74.70328057 |
| io 161 | 0.08140671 | 6.11974469 |
| io 162 | 0.40646449 | 23.70814384 |
| io 163 | 19.72272027 | 170.44853472 |
| io 165 | 15.83192674 | 198.35616243 |
| io 168 | 24.77472802 | 176.36505289 |
| io 172 | 13.38121586 | 201.90597211 |
| io 173 | 0.46374376 | 14.48458213 |
| io 174 | 2.13662149 | 33.42709641 |
| io 178 | 5.01133479 | 61.83270044 |
| io 179 | 5.85099454 | 71.52839628 |
| io 180 | 2.74793739 | 70.18325037 |
| io 182 | 2.29146239 | 80.88997339 |
| io 183 | 0.37544474 | 30.64093653 |
| io 186 | 4.37953149 | 139.27499665 |
| io 187 | 7.60228850 | 81.68098458 |
| io 191 | 1.55195055 | 49.81525772 |
| io 192 | 0.11136013 | 6.22326436 |
| io 202 | 0.72655233 | 20.43935318 |
| io 211 | 2.71494549 | 24.11026016 |
| io 212 | 0.75392961 | 13.07604289 |
| io 213 | 1.12146690 | 29.22385647 |
| io 214 | 1.28388080 | 46.99541429 |
| io 215 | 1.22984757 | 30.24580767 |
| io 218 | 7.38712290 | 72.05692274 |
| io 219 | 2.85509175 | 82.29106504 |
| io 222 | 3.05610029 | 151.66292867 |
| io 224 | 2.42779101 | 48.65060565 |
| io 225 | 1.57627189 | 39.92990640 |
| io 232 | 1.99257599 | 42.78019475 |
| io 241 | 0.05518481 | 4.32167118 |
| io 246 | 4.09721249 | 56.06843583 |
| io 249 | 2.25915511 | 40.54325768 |
| io 250 | 0.05503556 | 3.68110831 |
| io 256 | 0.72516558 | 27.51467487 |
| io 258 | 0.79983191 | 20.26407797 |
| io 264 | 0.76265583 | 20.91930047 |
| io 272 | 0.35362097 | 22.83911537 |
| io 274 | 2.21739739 | 66.72062792 |
| io 276 | 0.16598963 | 11.62852022 |
| io 286 | 16.35137975 | 372.92534743 |
| io 294 | 0.62522811 | 22.62024958 |
| io 297 | 9.92812369 | 165.34368819 |
| io 299 | 0.84535649 | 32.33607148 |
| io 309 | 0.14876733 | 7.72623402 |
| io 319 | 0.55427931 | 13.79992162 |
| io 331 | 0.02521362 | 2.03521531 |
| io 336 | 1.15142177 | 51.99660482 |
| io 346 | 0.37085026 | 17.65801997 |
| io 353 | 0.43987545 | 13.44523405 |
| io 362 | 3.47473386 | 66.22076443 |
| io 367 | 2.70948312 | 95.55443337 |
| io 370 | 1.28904608 | 62.02432101 |
| io 372 | 0.23394400 | 10.66215486 |
| io 375 | 1.96216241 | 38.09575102 |
| io 388 | 0.24513237 | 13.13709014 |
| io 393 | 1.35958993 | 31.38702890 |
| io 397 | 0.46400056 | 22.54992593 |
| io 398 | 1.19107485 | 37.87508913 |
| io 406 | 0.20310968 | 18.16141186 |
| io 411 | 0.07567586 | 4.21071036 |
| io 414 | 0.32353971 | 14.14960725 |
| io 415 | 0.22762292 | 13.42311488 |
| io 436 | 0.33718833 | 26.49641673 |
| io 454 | 0.11939764 | 4.58772626 |
| io 473 | 4.31411962 | 176.19192715 |
| io 474 | 0.13252907 | 10.94617428 |
| io 477 | 0.34700238 | 16.02948289 |
| io 481 | 5.82171873 | 180.48098711 |
| io 491 | 0.25213615 | 11.31837615 |
| io 496 | 0.34278265 | 14.85375749 |
| io 518 | 0.73819863 | 30.68261071 |
| io 537 | 0.29415885 | 9.56261768 |
| io 541 | 0.38709204 | 23.46093078 |
| io 555 | 0.25756408 | 19.97592587 |
| io 563 | 2.98907819 | 133.68862961 |
| io 580 | 0.88948032 | 28.94821707 |
| io 591 | 7.79520941 | 488.69346735 |
| io 615 | 1.40565906 | 63.71466453 |
| io 648 | 0.07984312 | 5.43627512 |
| io 659 | 0.18679087 | 11.14743115 |
| io 703 | 0.81881216 | 47.93904756 |
| io 715 | 1.98718308 | 106.67294807 |
| io 761 | 0.44081733 | 19.61930125 |
| io 781 | 0.38380726 | 24.10652565 |
| io 898 | 1.05757109 | 63.36826128 |
| io 900 | 1.10827030 | 32.44354405 |
| io 926 | 3.21893823 | 86.65195228 |
| io 1101 | 0.25213615 | 15.51705456 |
| io 1683 | 3.73966942 | 163.04782953 |
| io 1698 | 0.03992156 | 3.08545750 |
| io 1707 | 0.81923239 | 23.84415661 |
| io 1867 | 0.20755708 | 9.68891084 |
| io 1883 | 0.69227339 | 25.56599050 |
| io 1898 | 0.32028295 | 14.07166623 |
| io 3680 | 2.17134753 | 97.91951352 |
| io 3692 | 0.67297092 | 23.45302190 |
| io 4691 | 0.16759613 | 10.00683424 |
| io 4692 | 0.03291602 | 2.16073240 |
| med 25 | 46.81337278 | 216.13348495 |
| med 47 | 0.00158135 | 0.12283066 |
| med 49 | 0.41836614 | 7.36927477 |
| med 115 | 0.00433139 | 0.20011319 |
| med 118 | 0.63889367 | 9.07459691 |
| med 120 | 0.00212875 | 0.05821205 |
| med 123 | 0.02503612 | 0.55712674 |
| med 126 | 0.05372515 | 1.14193471 |
| med 127 | 0.03595630 | 1.75111724 |
| med 133 | 0.24830962 | 3.46886288 |
| med 134 | 0.00405895 | 0.19650777 |
| med 163 | 0.00136277 | 0.10707113 |
| totalbal 1 | 814.25867763 | 1516.14917639 |
| totalbal 2 | 598.30432761 | 1021.08972972 |
| totalbal 3 | 55.96514866 | 246.98815463 |
| totalbal 4 | 0.48738546 | 9.22563007 |
| totalbal 5 | 15.11870912 | 81.97208961 |
| totalbal 6 | 5.11092240 | 54.36397034 |
| totalbal 7 | 5.82342626 | 91.33320416 |
| totalbal 8 | 1.43187293 | 28.48242708 |
| totalbal 9 | 0.25374235 | 6.66952028 |
| totalbal 10 | 0.04412383 | 3.07006251 |
| totalbal 16 | 13.57840095 | 79.02372621 |
| totalbal 18 | 441.86617120 | 815.10281506 |
| totalbal 19 | 1.77952589 | 37.89367554 |
| totalbal 20 | 140.43823982 | 296.05436437 |
| totalbal 23 | 18.36761510 | 62.36754817 |
| totalbal 24 | 4.99583432 | 58.63717272 |
| totalbal 25 | 8.33850329 | 151.97044567 |
| totalbal 26 | 488.44715335 | 820.26058063 |
| totalbal 27 | 219.09744385 | 953.56024176 |
| totalbal 28 | 434.20308217 | 1767.96178280 |

### Machine Learning on Data

Machine learning was carried out with the scikit.learn package under Python language version 2.7 running in the Windows operating system within the environment Anaconda. In addition, we used the statistical software package R 3.1.2 (64-bit version) under Windows to perform tasks in data preparation and analysis. This scikit.learn package version 0.16.0 is designed to produce machine learning models for the purpose of classification and regression on dense and sparse datasets. The following classifiers were used: Nearest Neighbors, Linear SVM (support vector machine), RBF SVM (radial basis function support vector machine), Decision Trees, Random Forest (RF), AdaBoost, Naive Bayes, and Logistic Regression (LR). The best classifier can be selected through model and parametric optimization. For some applications the best classifier might be that with the highest accuracy among all the classifiers tested. For other applications the best classifier might be the one with the highest positive predictive value (PPV), negative predictive value (NPV), specificity, selectivity, area under the curve (AUC), as defined below, or some other combination of performance attributes. For the examples presented here, accuracy was generally used to rate classifiers. Because the Logistic Regression performed very well, the machine learning results presented here use it unless otherwise stated. Although the foregoing is what we used for our work, a person of ordinary skill in the art would readily appreciate that many other machine learning concepts and algorithms could equally be used and applied in the methods of the invention, including but not limited to artificial neural networks (ANN), Bayesian statistics, case-based reasoning, Gaussian process regression, inductive logic programming, learning automata, learning vector quantization, informal fuzzy networks, conditional random fields, genetic algorithms (GA), Information Theory, support vector machine (SVM), Averaged One-Dependence Estimators (AODE), Group method of data handling (GMDH), instance-based learning, lazy learning, and Maximum Information Spanning Trees (MIST). Moreover, various forms of boosting can be applied with combinations of methods.

Some of these learning methods require additional parameters to run. For the complexity parameter in SVM and LR in separate runs we used values ranging from 0.0001 to 1000 by powers of ten. The same set of values were also applied to the gamma parameter in RBF SVM. The Decision Tree method was used with a maximum depth of 10, Adaboost had a minimum number of estimators equal to 50, and RF had a minimum of 50 estimators.

In a typical machine learning calculation set, and as we used here, independent of which classifier was being used, the original dataset was split in a random fashion into 2 datasets: a training dataset and a testing dataset, with the training dataset containing a random 80% of the data instances (an individual patient acquiring SIRS at a specific time [positive] or not [negative]) and the testing dataset containing the remaining 20% of the data. This separation of training from testing data is typical in supervised machine learning applications, so that the model developed in the training phase can be evaluated in the testing phase on data to which it has not previously been exposed (e.g., the testing data is equivalent to patients to whom the model has no exposure initially, but the model will make predictions about those patients after exposure to the training data, and then those predictions can be evaluated by comparing them to the testing data itself that represents those patients).

For each of these classifiers, the model parameters that determine their predictive model were computed on the basis of the training dataset. For the logistic regression results reported here, the parameters for each resulting model are one coefficient for each data feature in the model plus a single bias value. A data feature is a type of measurement (systolic blood pressure measurement, for example). As shown in the equation below, a linear combination of coefficients (wⱼ) and normalized data features (patient_data_{i,j}), together with the bias (b) produces the prediction.

Each classifier model was then used, with its own respective set of parameters obtained from the training dataset (as described above), and was evaluated on the testing dataset and prediction results were expressed in the form of accuracy, positive predictive value (PPV), sensitivity, specificity, negative predictive value (NPV), and area under the curve (AUC), as defined below.

The logistic regression was selected for its excellent accuracy, positive predictive value and its robustness. See Yu et al. (2011). Several random combinations of training and test datasets were used to reproduce the results. This strategy was used to eliminate the possibility that results were due to a serendipitous selection of the test dataset. The logistic regression model results presented here were run with complexity parameter set equal to 0.005 and penalty L2.

Predictions are made from the logistic regression model using the following equation:

$$P \left(S I R S \vert {\text{patient\_data}}_{i}\right) = \frac{1}{1 + \text{exp} \left(- b - {\sum }_{j = 1}^{\text{num\_features}} {w}_{j} \times {\text{patient\_data}}_{i , j}\right)}$$

where P(SIRS|patient_dataᵢ) is the probability that a particular patient i presenting normalized patient data represented by the vector patient_dataᵢ will develop SIRS at the corresponding time point in the model, given the model bias parameter b and model coefficients Wⱼ corresponding to the normalized patient feature measurements (of which there are num_features, indexed by j) patient_data_{i,j}.

In the work presented here a probability of greater than 50% (one-half) results in a prediction of the patient having SIRS at the corresponding future time point, and a probability less than or equal to 50% (one-half) is a prediction of not having SIRS. As one of ordinary skill in the art would appreciate, it is straightforward to apply more sophisticated treatments of this probability to assign finer grained priorities to the possibility and severity of a condition. For example, one could use the probability directly as a measure of the predicted probability of developing SIRS, where, rather than a binary prediction of which patients will or will not develop SIRS, one could map the probabilities to categories such as “highly likely to develop SIRS,” “probably will develop SIRS,” “could develop SIRS,” “unlikely to develop SIRS,” and “highly unlikely to develop SIRS.” These finer grained priorities may be especially useful to hospitals in taking action on the predictions.

### Feature Selection

A machine learning algorithm can be used to generate a prediction model based on a patient population dataset. However, there is a tremendous amount of data in the patient population dataset, much of which is not necessary or provides little contribution to the predictability of a particular disease for which the prediction model is being trained. Additionally, it is often the case that different particular patients only have available data for different respective subsets of all of the features of the datasets, so that a prediction model based on all of the features of the patient population dataset might not be usable for particular patients or might output suboptimal predictions for the particular patients. An example embodiment of the present invention identifies a plurality of subsets of features within the totality of features of the patient population dataset for which to produce respective prediction models, that can be used to predict a disease, e.g., SIRS, based on data of only the respective subset of features.

Thus, in an example embodiment of the present invention, a computer system is provided with a patient population dataset, from which the system selects a plurality of subsets, each subset being used by a machine learning algorithm, which is applied by the system to the respective subset, to train a new prediction model on the basis of which to predict for a patient onset of a disease, e.g., SIRS. Thus, for each selected subset, a respective prediction model can be trained, with each of the trained prediction models being subsequently applied to an individual patient’s data with respect to the particular group of features of the subset for which the respective prediction model had been trained.

Thus, according to the example embodiment, in a preliminary selection step, a feature selection method is applied to select relevant subsets of features for training respective prediction models. In an example embodiment, prior to application of the feature selection method (or, viewed differently, as a first step of the feature selection method), features are initially removed from the dataset based on Bhattacharyya distance as described above. Then, from those features not removed based on the Bhattacharyya distance, the system proceeds to select groups of relevant features to which to apply a machine learning algorithm, where the machine learning algorithm would then generate a respective prediction model based on data values of the selected relevant features of each of one or more of the groups.

The feature selection method includes computing the correlation between each feature at a given time point and the output array (-1 for negatives [patients who had not developed SIRS]; +1 for positives [patients who had developed SIRS at the target time]), and computing the correlation between all pairs of features at a given time point. Iteratively, a feature was selected as a primary feature at a time point if it had the greatest correlation with the output array amongst all of the remaining features for that time point (6, 12, 24, or 48 hours). Then, for that time point, all others of the remaining features that had a correlation of 60% or greater (when taken across patients) with the most recently selected primary feature at that time point (i.e., the primary feature selected for the present iteration) were selected as a secondary feature associated with that primary feature and time point. For example, in an example embodiment, for each feature, a vector is generated populated with a value for the respective feature for each of a plurality of patients of a patient population, and correlation is determined between the vector of the selected primary feature and the remaining feature vectors. The vectors can further be indicated to be associated with negatives or with positives.

All secondary features thus selected were then removed from the set of remaining features (so that once a feature is selected as a primary or secondary feature, it can no longer be selected as a primary feature in a subsequent iteration). This selected primary feature and its associated secondary features were together considered a feature group. Because of the method used to select a feature group, the members of a particular feature group had some correlation with the output (whether patients developed SIRS at a specific time in the future) and some correlation amongst themselves. Thus, they are expected to be useful in the prediction of SIRS, but members within a feature group might be partially redundant owing to their correlation amongst themselves. This process was repeated iteratively, first picking an additional primary feature at the same time point and then its associated secondary features at that time point (which together produced an additional feature group).

In an example embodiment, the iterative feature selection method is discontinued as soon as it is determined that the remaining unselected features have essentially no predictive power, as indicated by machine learning, e.g., with an AUC very close to 0.50 (such as 0.50 ± 0.05). For example, the system selects a primary feature and its secondary features as a new feature subset. The system then applies machine learning to the combination of all of the remaining features of the patient population dataset. If the machine learning produces an operable prediction model based on those remaining features, then the system continues on with another iteration to find one or more further subsets of those remaining features that can be used alone. On the other hand, if the machine learning does not produce an operable prediction model based on those remaining features, then the iterative selection method is ended. Once the iterative selection method is ended, the system applies a machine learning algorithm to each of one or more, e.g., each of all, of the individual feature subsets that had been selected by the iterative feature selection method to produce respective prediction models.

In an example embodiment, this process is carried out separately for each of a plurality of values of a particular constraint, e.g., time points. For example, in an example, this method was performed for each of the noted four onset time points of 6, 12, 24, and 48 hours.

As one of ordinary skill in the art would readily appreciate, the above Machine Learning on Data and Feature Selection methods were carried out using the MIMIC II database, but the same methods of the invention could be utilized on another database from other hospitals to achieve the results of the invention, including identification of primary, secondary and additional features, exemplified here with the MICMI II database.

The feature selection method was applied to the entire patient population dataset. Once the relevant features were selected in this manner, the patient population dataset was divided into the training dataset and the testing dataset for performing the training and testing steps.

### Example 1: Machine Learning Results Show Predictive Value of Selected Features

The performance results for machine learning with the linear support vector machine method (with the complexity parameter C=0.001) using data associated with the features in Table 4 are shown in Table 2. Results for four separate sets of calculations are presented in this Table 2, each set corresponding to a respective onset time period. For each of the respective onset time periods, the table shows results of a calculation generated based on features grouped as primary and secondary features in the center column and results of calculations generated based on “remaining features” that were not removed in the Bhattacharyya procedure. The results show that the former calculations are predictive and the latter calculations are not predictive. Table 3 shows further details of this “remaining features” set for the 48-hour dataset. Each calculation used a different set of data (collected 6, 12, 24, or 48 hours in advanced of the onset of SIRS for the positive patients). For each of the four sets of calculations, the results show that using only the data associated with the features in Table 4, accurate predictions could be made regarding which patients would and which would not develop SIRS, as judged by statistical measures familiar to the machine learning community and a person of ordinary skill in the art, such as accuracy, sensitivity, specificity, positive predictive value (PPV), negative predictive value (NPV), and area under the curve (AUC). In addition, the true positive rate (TP), true negative rate (TN), false positive rate (FP), and false negative rate (FN) are given. Parallel experiments using only feature data that were associated with features not primary or secondary for the associated time points dataset and not removed by the Bhattacharyya procedure (“remaining features”) were unable to make accurate predictions regarding which patients would and which would not develop SIRS at the selected time, demonstrating the effectiveness of the invention. This is indicated by an area under the curve of very close to 50% when the remaining features were used.

The meanings for various terms in Table 2 (and also used elsewhere in this application) are standard in the machine learning literature and are well known to one of ordinary skill in the art, but are referenced here in exemplary form for the sake of completeness. True positives (TP) are patients, whether in the training set or testing set, and whether historical or prospective, who are predicted to develop SIRS (generally in a given time window) and who do subsequently develop SIRS (generally in that given time window). True negatives (TN) are patients predicted not to develop SIRS who do not subsequently develop SIRS. False positives (TP) are patients predicted to develop SIRS but who do not subsequently develop SIRS, and false negatives (FN) are patients predicted to not develop SIRS but who subsequently do develop SIRS. Among any set of patients for whom predictions are made, the accuracy statistic is the total number of correct predictions divided by the total number of predictions made. Thus, accuracy can be represented as (TP+TN)/(TP+FP+TN+FN). Accuracy, and the other statistics described and used here, are often represented as a percentage (by multiplying by 100 and adding the percentage symbol). Sensitivity is the fraction of patients who subsequently develop SIRS who are correctly predicted, and can be represented as TP/(TP+FN). Specificity is the fraction of patients who subsequently do not develop SIRS who are correctly predicted, and can be represented as TN/(TN+FP). Positive predictive value (PPV) is the fraction of positive predictions that are correct, and can be represented as TP/(TP+FP). Negative predictive value (NPV) is the fraction of negative predictions that are correct, and can be represented as TN/(TN+FN). Area under the curve (AUC) is the area under the receiver operating characteristic (ROC) curve, which is a plot of sensitivity, on the y-axis, against (1-specificity), on the x-axis, as the discrimination threshold is varied. It is a non-negative quantity whose maximum value is one. Different machine learning methods have their own mechanism of varying the discrimination threshold. In logistic regression that can be achieved by changing the threshold probability between calling a prediction negative and positive (nominally 0.5), for example by progressively varying it from zero to one, which then maps out the ROC curve.

Together, this evidence shows that the features in Table 4 have value in machine learning prediction of patients who will and who will not develop SIRS in the next 6-to-48 hours, and that other features in the dataset do not. That is, these features selected by the feature selection method described above, when applied to a machine learning algorithm, cause the machine learning algorithm to generate a good prediction model, the prediction model accepting patient-specific values for those selected features to predict likelihood of the respective specific patients developing SIRS.

TABLE 2

Results for four separate sets of calculations are presented. Each calculation used data from a different set of data from MIMIC II (collected 6, 12, 24, or 48 hours in advanced of the onset of SIRS for the positive patients). For each of the four sets of calculations, the results show that using only the data associated with the features in Table 4 (all primary and secondary features associated with that time point), accurate predictions could be made regarding which patients would and which would not develop SIRS, as judged by statistical measures familiar to the machine learning community such as accuracy, sensitivity, specificity, positive predictive value (PPV), negative predictive value (NPV), and area under the curve (AUC). In addition, the true positive rate (TP), true negative rate (TN), false positive rate (FP), and false negative rate (FN) are given. Parallel experiments using only data that were associated with features not primary or secondary for that time point (“remaining features”) were unable to make accurate predictions regarding which patients would and which would not develop SIRS at the selected time This is indicated by an area under the curve of very close to 50%

| | | |
|---|---|---|
| | Using only primary and secondary features at time point | Using only remaining features (not primary or secondary) at time point |
| 6-hour dataset | Accuracy=82.01% Sensitivity=90.87% Specificity=54.80% PPV=86.07% NPV=66.14% TP=68.56% TN=13.45% FP=11.09% FN=6.88% AUC=72.84% | Specificity=0.30% AUC=50.03% |
| 12-hour dataset | Accuracy=81.29% Sensitivity=87.09% Specificity=66.34% PPV=86.96% NPV=66.60% | Specificity=4.79% AUC=50.91% |
| | TP=62.75% TN=18.53% FP=9.40% FN=9.29% AUC=76.71% | |
| 24-hour dataset | Accuracy=82.02% Sensitivity=85.96% Specificity=73.81% PPV=87.26% NPV=71.57% TP=58.12% TN=23.90% FP=8.47% FN=9.49% AUC=79.88% | Specificity=0.0% AUC=50.0% |
| 48-hour dataset | Accuracy=84.55% Sensitivity=84.73% Specificity=84.26% PPV=90.10% NPV=76.54% TP=53.25% TN=31.30% FP=5.84% FN=9.59% AUC=84.49% | Specificity=0.0% AUC=50.0% |

An example of a poorly predictive model with area under the curve of 50% is shown in Table 3 (48-hour data, remaining features only [not primary features for 48-hour data, not secondary features for 48-hour data, and not features removed in the Bhattacharyya procedure]). Most of the features have a coefficient value of zero, indicative of model that had difficulty learning from the training data. This indicates that the remaining features set, listed in the Table 3, was not sufficiently informative to predict SIRS occurrence.

TABLE 3

This is an example of a model that is poorly predictive of SIRS occurrence. The features are listed in the first column (except for the first row, which contains the model bias), and the coefficients Wⱼ in the model are shown in the third column. The middle column contains a brief description of each feature

| Feature ID | Brief Feature Description | Coefficient Value |
|---|---|---|
| Bias chart 2 | (b in model) ABI(L) | 0.182399577 0 |
| chart 4 | ABI Ankle BP R/L | 0 |
| chart 5 | ABI Brachial BP R/L | 0 |
| chart 25 | AV Interval | 0 |
| chart 26 | AaDO2 | 0 |
| chart 29 | Access mmHg | 0 |
| chart 63 | BIPAP - BPM | 0 |
| chart 65 | BIPAP - Est. Vt | 0 |
| chart 79 | Blood Flow ml/min | 0 |
| chart 92 | CPP | 0 |
| chart 142 | Current Goal | 0 |
| chart 146 | Dialysate Flow ml/hr | 0 |
| chart 181 | Epidural Total Dose | 0 |
| chart 186 | FIO2 Alarm-Low | 0 |
| chart 192 | Filter Pressure mmHg | 0 |
| chart 221 | I:E Ratio | 0 |
| chart 226 | ICP | 0 |
| chart 440 | MDI #2 (Puff/Drug) | 0 |
| chart 441 | MDI #3 (Puff/Drug) | 0 |
| chart 442 | Manual BP | 0 |
| chart 449 | Minute Volume (Set) | 0 |
| chart 472 | O2AV | 0 |
| chart 473 | O2AVI | 0 |
| chart 481 | Orthostat HR sitting | 0 |
| chart 482 | OrthostatBP standing | 0 |
| chart 483 | OrthostatHR standing | 0 |
| chart 484 | Orthostatic BP lying | 0 |
| chart 485 | Orthostatic HR lying | 0 |
| chart 490 | PAO2 | 0 |
| chart 491 | PAP Mean | 0 |
| chart 492 | PAP S/D | 0 |
| chart 494 | PCA Basal Rate | 0 |
| chart 496 | PCA Dose | 0 |
| chart 498 | PCA Lockout (Min) | 0 |
| chart 503 | PCV Set Insp. Press | 0 |
| chart 512 | PVR | 0 |
| chart 517 | Pacer Rate | 0 |
| chart 595 | RSBI (<200) | 0 |
| chart 601 | RVSW | 0 |
| chart 602 | RVSWI | 0 |
| chart 607 | Rec.Breath Time(sec) | 0 |
| chart 624 | Return Pressure mmHg | 0 |
| chart 626 | SVR | 0 |
| chart 664 | Swan SVO2 | 0 |
| chart 670 | TCPCV Insp. Pressure | 0 |
| chart 671 | TCPCV Insp. Time | 0 |
| chart 686 | Total PEEP Level | 0 |
| chart 725 | Vision - IPAP | 0 |
| chart 727 | Vision FiO2 | 0 |
| chart 773 | Alk. Phosphate | 0 |
| chart 784 | CPK | 0 |
| chart 792 | Cyclosporin | 0 |
| chart 793 | D-Dimer (0-500) | 0 |
| chart 809 | Gentamycin/Random | 0 |
| chart 817 | LDH | 0 |
| chart 826 | Phenobarbital | 0 |
| chart 835 | Sed Rate | 0 |
| chart 836 | Serum Osmolality | 0 |
| chart 844 | Thrombin (16-21) | 0 |
| chart 850 | Triglyceride (0-200) | 0 |
| chart 851 | Troponin | 0 |
| chart 856 | Vancomycin/Trough | 0 |
| chart 1223 | HIGH EXHALED MIN VOL | 0 |
| chart 1340 | high minute volume | 0 |
| chart 1390 | DO2 | 0 |
| chart 1391 | DO2I | 0 |
| chart 1397 | RSBI (<100) | 0 |
| chart 1401 | zzO2AV | 0 |
| chart 1402 | zzO2AVI | 0 |
| chart 1411 | Bladder Pressure | 0 |
| chart 1486 | High Minute Volume | 0 |
| chart 1520 | ACT | 0 |
| chart 1524 | Cholesterol | 0 |
| chart 1526 | D-Dimer | 0 |
| chart 1528 | Fibrinogen | 0 |
| chart 1537 | Thrombin | 0 |
| chart 1540 | Triglyceride | 0 |
| chart 1546 | high ve | 0 |
| chart 1565 | High MV Limit | 0 |
| chart 1624 | PreSep Catheter SVO2 | 0 |
| chart 1671 | act | 0 |
| chart 2139 | EDVI | 0 |
| chart 5683 | Hourly PFR | 0 |
| chart 5816 | ICP Alarm (Lo/Hi) | 0 |
| chart 5818 | PAP Alarm (Lo/Hi) | 0 |
| chart 6702 | Arterial BP Mean #2 | 0 |
| chart 6711 | INV#5 Cap Change | 0 |
| chart 6712 | INV#5 Tubing Change | 0 |
| lab 50001 | AADO2 | 0 |
| lab 50013 | O2 | 0 |
| lab 50038 | AMYLASE | 0 |
| lab 50042 | CREAT | 0 |
| lab 50044 | LD(LDH) | 0 |
| lab 50055 | %phenyfr | 0 |
| lab 50056 | ACETMNPHN | 0 |
| lab 50059 | AFP | 0 |
| lab 50061 | ALK PHOS | 0 |
| lab 50062 | ALT(SGPT) | 0 |
| lab 50064 | AMMONIA | 0 |
| lab 50071 | ANTITPO | 0 |
| lab 50072 | ASA | 0 |
| lab 50073 | AST(SGOT) | 0 |
| lab 50075 | C3 | 0 |
| lab 50076 | C4 | 0 |
| lab 50077 | CA125 | 0 |
| lab 50078 | CA27.29 | 0 |
| lab 50082 | CEA | 0 |
| lab 50086 | CK(CPK) | 0 |
| lab 50087 | CK-MB | 0 |
| lab 50089 | CORTISOL | 0 |
| lab 50093 | CYCLSPRN | 0 |
| lab 50094 | DHEA-SO4 | 0 |
| lab 50098 | ESTRADL | 0 |
| lab 50099 | ETHANOL | 0 |
| lab 50101 | FERRITIN | 0 |
| lab 50102 | FK506 | 0 |
| lab 50106 | FSH | 0 |
| lab 50107 | GASTRIN | 0 |
| lab 50109 | GGT | 0 |
| lab 50115 | HAPTOGLOB | 0 |
| lab 50120 | HCG | 0 |
| lab 50129 | IgA | 0 |
| lab 50130 | IgG | 0 |
| lab 50138 | LIPASE | 0 |
| lab 50144 | OSMOLAL | 0 |
| lab 50146 | PHENOBARB | 0 |
| lab 50152 | PROLACTIN | 0 |
| lab 50154 | PTH | 0 |
| lab 50158 | RHEU FACT | 0 |
| lab 50164 | T4Index | 0 |
| lab 50165 | TESTOSTER | 0 |
| lab 50167 | THYROGLB | 0 |
| lab 50173 | TRF | 0 |
| lab 50179 | VALPROATE | 0 |
| lab 50181 | VIT B 12 | 0 |
| lab 50190 | calTIBC | 0 |
| lab 50195 | proBNP | 0 |
| lab 50196 | rapamycin | 0 |
| lab 50202 | LD(LDH) | 0 |
| lab 50204 | PROTEIN | 0 |
| lab 50208 | GLUCOSE | 0 |
| lab 50212 | AMYLASE | 0 |
| lab 50216 | CREAT | 0 |
| lab 50217 | GLUCOSE | 0 |
| lab 50218 | LD(LDH) | 0 |
| lab 50225 | POTASSIUM | 0 |
| lab 50226 | SODIUM | 0 |
| lab 50232 | UREA N | 0 |
| lab 50235 | AMYLASE | 0 |
| lab 50237 | CHOLEST | 0 |
| lab 50239 | CREAT | 0 |
| lab 50240 | GLUCOSE | 0 |
| lab 50241 | LD(LDH) | 0 |
| lab 50247 | TRIGLYCER | 0 |
| lab 50250 | OSMOLAL | 0 |
| lab 50251 | POTASSIUM | 0 |
| lab 50252 | SODIUM | 0 |
| lab 50253 | 24Ca++ | 0 |
| lab 50254 | 24Creat | 0 |
| lab 50255 | 24Prot | 0 |
| lab 50258 | <CREAT-U> | 0 |
| lab 50259 | <VOL-U> | 0 |
| lab 50260 | AMY/CREAT | 0 |
| lab 50261 | AMYLASE | 0 |
| lab 50263 | CHLORIDE | 0 |
| lab 50265 | CREAT CLR | 0 |
| lab 50266 | GLUCOSE | 0 |
| lab 50273 | PHOSPHATE | 0 |
| lab 50276 | PROT/CREA | 0 |
| lab 50277 | SODIUM | 0 |
| lab 50278 | TOT PROT | 0 |
| lab 50279 | TOTAL CO2 | 0 |
| lab 50284 | URIC ACID | 0 |
| lab 50285 | VOLUME | 0 |
| lab 50287 | alb/CREA | 0 |
| lab 50288 | albumin | 0 |
| lab 50302 | HCT | 0 |
| lab 50304 | MACROPHAG | 0 |
| lab 50313 | RBC | 0 |
| lab 50314 | WBC | 0 |
| lab 50317 | ABS CD3 | 0 |
| lab 50318 | ABS CD4 | 0 |
| lab 50319 | ABS CD8 | 0 |
| lab 50320 | ABS LYMPH | 0 |
| lab 50322 | ACA IgG | 0 |
| lab 50323 | ACA IgM | 0 |
| lab 50330 | AT III | 0 |
| lab 50335 | BLASTS | 0 |
| lab 50356 | CD4 | 0 |
| lab 50357 | CD4/CD8 | 0 |
| lab 50367 | CD8 | 0 |
| lab 50374 | EOS CT | 0 |
| lab 50378 | FIBRINOGE | 0 |
| lab 50382 | GRAN CT | 0 |
| lab 50385 | HEPARIN | 0 |
| lab 50390 | HGBF | 0 |
| lab 50395 | HYPERSEG | 0 |
| lab 50404 | LAP | 0 |
| lab 50427 | PLASMA | 0 |
| lab 50428 | PLT COUNT | 0 |
| lab 50434 | PROMYELO | 0 |
| lab 50436 | PROT C FN | 0 |
| lab 50437 | PROT S AG | 0 |
| lab 50438 | PROT S FN | 0 |
| lab 50441 | QUAN G6PD | 0 |
| lab 50451 | SED RATE | 0 |
| lab 50460 | THROMBN | 0 |
| lab 50461 | V | 0 |
| lab 50463 | VIII | 0 |
| lab 50465 | VWF AG | 0 |
| lab 50466 | VWF CO | 0 |
| lab 50469 | X | 0 |
| lab 50473 | YOUNG | 0 |
| lab 50510 | BANDS | 0 |
| lab 50511 | BASOS | 0 |
| lab 50513 | EOS | 0 |
| lab 50526 | RBC | 0 |
| lab 50530 | BANDS | 0 |
| lab 50537 | LYMPHS | 0 |
| lab 50541 | MONOS | 0 |
| lab 50545 | POLYS | 0 |
| lab 50546 | RBC | 0 |
| lab 50548 | WBC | 0 |
| lab 50549 | ATYPS | 0 |
| lab 50550 | BANDS | 0 |
| lab 50551 | BASOS | 0 |
| lab 50560 | CD19 | 0 |
| lab 50565 | CD3 | 0 |
| lab 50567 | CD34 | 0 |
| lab 50579 | EOS | 0 |
| lab 50587 | LYMPHS | 0 |
| lab 50588 | MACROPHAG | 0 |
| lab 50589 | MESOTHELI | 0 |
| lab 50598 | RBC | 0 |
| lab 50599 | WBC | 0 |
| lab 50600 | ATYPS | 0 |
| lab 50603 | EOS | 0 |
| lab 50604 | HCT | 0 |
| lab 50609 | MONOS | 0 |
| lab 50614 | POLYS | 0 |
| lab 50616 | RBC | 0 |
| lab 50617 | WBC | 0 |
| lab 50632 | CELL | 0 |
| lab 50641 | GLUCOSE | 0 |
| lab 50647 | KETONE | 0 |
| lab 50652 | NSQ EPI | 0 |
| lab 50655 | PROTEIN | 0 |
| lab 50659 | RENAL EPI | 0 |
| lab 50664 | TRANS EPI | 0 |
| lab 50675 | WBCCAST | 0 |
| lab 50687 | MS-AFP | 0 |
| lab 50689 | MS-HCG | 0 |
| lab 50690 | MS-UE3 | 0 |
| lab 50699 | tacroFK | 0 |
| io 48 | Chest Tubes Left Pleural 1 | 0 |
| io 49 | Chest Tubes Right Pleural 1 | 0 |
| io 51 | Gastric Gastric Tube | 0 |
| io 52 | Gastric Nasogastric | 0 |
| io 53 | Stool Out Fecal Bag | 0 |
| io 58 | Cerebral Drain R Ventricular Drain | 0 |
| io 59 | Gastric Oral Gastric | 0 |
| io 60 | Pre-Admission Output Pre-Admission Output | 0 |
| io 61 | OR Out OR Urine | 0 |
| io 63 | Stool Out Ileostomy | 0 |
| io 64 | OR Out EBL | 0 |
| io 65 | OR Out PACU Urine | 0 |
| io 66 | Drain Out #1 Tap | 0 |
| io 68 | Stool Out Other | 0 |
| io 70 | Drain Out #1 Hemovac | 0 |
| io 71 | Drain Out # 1 Jackson Pratt | 0 |
| io 72 | Drain Out #2 Jackson Pratt | 0 |
| io 73 | PACU Out PACU Drains | 0 |
| io 74 | PACU Out PACU NG | 0 |
| io 76 | Chest Tubes CTICU CT 1 | 0 |
| io 77 | Drain Out #1 Pericardial | 0 |
| io 80 | Drain Out #2 Other | 0 |
| io 84 | Chest Tubes Other | 0 |
| io 85 | Urine Out Incontinent | 0 |
| io 87 | Stool Out Colostomy | 0 |
| io 88 | Drain Out #3 Jackson Pratt | 0 |
| io 91 | Chest Tubes Mediastinal | 0 |
| io 92 | Drain Out #4 Jackson Pratt | 0 |
| io 93 | Drain Out #1 T Tube | 0 |
| io 94 | Urine Out Condom Cath | 0 |
| io 104 | D5W 100.0 ml | -0.029107698 |
| io 106 | Lactated Ringers | 0 |
| io 107 | 0.9% Normal Saline | 0 |
| io 123 | OR Colloid | 0 |
| io 124 | OR Crystalloid | 0 |
| io 125 | PACU Crystalloids | 0 |
| io 128 | TF Residual | 0 |
| io 130 | D5/.45NS | 0 |
| io 131 | D5/.45NS 10000.0 ml | 0 |
| io 132 | D5W 50.0 ml | 0 |
| io 137 | D5W 250.0 ml + 25000Uhr Heparin | 0 |
| io 138 | D5W 125.0 ml + 125 mghr Diltiazem | 0 |
| io 139 | Sterile Water 100.0 ml | 0 |
| io 140 | D5W 250.0 ml + 400 mcgkgmin Dopamine | 0 |
| io 141 | N/A 50.0 vl + 500 mcgkgmin Propofol | 0 |
| io 142 | Lactated Ringers 1000.0 ml | 0 |
| io 143 | Dextrose 10% | 0 |
| io 144 | Packed RBC’s | 0 |
| io 147 | D5W 250.0 ml + 100 mcgmin Nitroglycerine | 0 |
| io 149 | N/A 100.0 vl + 1000 mcgkgmin Propofol | 0 |
| io 151 | .45% Normal Saline 1000.0 ml | 0 |
| io 152 | D5/.45NS 1000.0 ml | 0 |
| io 154 | D5NS | 0 |
| io 155 | Gastric Meds | 0 |
| io 158 | OR FFP | 0 |
| io 159 | PACU Colloids | 0 |
| io 161 | D5W 250.0 ml + 60 mcgmin Neosynephrine | 0 |
| io 162 | D5W 200.0 ml + 20 mghr Ativan | 0 |
| io 163 | Fresh Frozen Plasma | 0 |
| io 165 | D5W 1000.0 ml | 0 |
| io 168 | D5W | 0 |
| io 172 | OR Packed RBC’s | 0 |
| io 173 | D5W 100.0 ml + 100 mghr Morphine Sulfate | 0 |
| io 174 | D5W 250.0 ml + 600 mgmin Amiodarone | 0 |
| io 178 | D5W 250.0 ml + 50 mcgkgmin Nitroprusside | 0 |
| io 179 | Platelets | 0 |
| io 180 | 0.45% Normal Saline | 0 |
| io 182 | Nepro | 0 |
| io 183 | PPN | 0 |
| io 186 | TPN 1000.0 ml | 0 |
| io 187 | 0.9% Normal Saline 500.0 ml | 0 |
| io 191 | Replete w/fiber | 0 |
| io 192 | Carrier 1000.0 ml | 0 |
| io 202 | D5W 250.0 ml + 2 mcgkgmin Epinephrine-k | 0 |
| io 211 | 0.9% Normal Saline 100.0 ml + 100Uhr Insulin | 0 |
| io 212 | D5W 250.0 ml + 12.5 mcgkgmin Aggrastat | 0 |
| io 213 | D5W 250.0 ml + 4 mcgkgmin Levophed-k | 0 |
| io 214 | D5 Normal Saline | 0 |
| io 215 | D5W 250.0 ml + 100 mcgkgmin Nitroprusside | 0 |
| io 218 | D5W 250.0 ml + 60 mcgkgmin Neosynephrine-k | 0 |
| io 219 | D5RL 1000.0 ml | 0 |
| io 222 | Impact w/fiber | 0 |
| io 224 | OR Platelets | 0 |
| io 225 | 0.9% Normal Saline 100.0 ml + 100 mgmin Labetolol | 0 |
| io 232 | Albumin 5% | 0 |
| io 241 | D5W 250.0 ml + 100 mcgkgmin Cisatracurium + 100 mg kg hr Cisatracurium | 0 |
| io 246 | Hespan | 0 |
| io 249 | 0.9% Normal Saline 250.0 ml | 0 |
| io 250 | D5W 250.0 ml + 20 mcgkgmin Neosynephrine-k | 0 |
| io 256 | D5W 250.0 ml + 250 mcgkgmin Dobutamine | 0 |
| io 258 | Albumin 25% | 0 |
| io 264 | D5W 100.0 ml + 20 mcgkgmin Milrinone | 0 |
| io 272 | TPN w/Lipids | 0 |
| io 274 | Free Water Bolus | 0 |
| io 276 | D5W 200.0 ml | 0 |
| io 286 | Ultrafiltrate Ultrafiltrate | 0 |
| io 294 | Drain Out #1 Other | 0 |
| io 297 | D5NS 1000.0 ml | 0 |
| io 299 | D5 Normal Saline 1000.0 ml | 0 |
| io 309 | 0.9% Normal Saline 100.0 ml | 0 |
| io 319 | Cryoprecipitate | 0 |
| io 331 | Gastric Jejunostomy Tube | 0 |
| io 336 | Cell Saver | 0 |
| io 346 | Dextrose 10% 1000.0 ml | 0 |
| io 353 | D5W 100.0 ml + 100 mghr Lasix | 0 |
| io 362 | D5W 500.0 ml | 0 |
| io 367 | Stool Out Rectal Tube | 0 |
| io 370 | Tube Feeding | 0 |
| io 372 | D5W 250.0 ml + 16 mcgkgmin Levophed-k | 0 |
| io 375 | D5W 250.0 ml + 8 mcgkgmin Levophed-k | 0 |
| io 388 | Stool Out (non-specific) | 0 |
| io 393 | D5W 500.0 ml + 2 mgmin Lidocaine | 0 |
| io 397 | Washed PRBC’s | 0 |
| io 398 | Packed RBC’s 375.0 ml | 0 |
| io 406 | Gastric Other | 0 |
| io 411 | D5W 50.0 ml + 100 mghr Lasix | 0 |
| io 414 | 0.9% Normal Saline 200.0 ml + 200 mgmin Labetolol | 0 |
| io 415 | Carrier 250.0 ml | 0 |
| io 436 | D5W 300.0 ml + 1200 mgmin Labetolol | 0 |
| io 454 | D5W 250.0 ml + 9 mcgkgmin Reopro + 9 mcgmin Reopro | 0 |
| io 473 | Urine Out IleoConduit | 0 |
| io 474 | 0.9% Normal Saline 100.0 ml + 200 mgmin Labetolol | 0 |
| io 477 | Sterile Water 100.0 ml + 100 mgmin TPA | 0 |
| io 481 | Promote w/fiber | 0 |
| io 491 | PACU Out EBL | 0 |
| io 496 | D5W 250.0 ml + 120 mcgkgmin Neosynephrine-k | 0 |
| io 518 | D5 Ringers Lact. 1000.0 ml | 0 |
| io 537 | Cerebral Drain Subdural | 0 |
| io 541 | D5W 500.0 ml + 2 mcgkgmin Narcan | 0 |
| io 555 | Promote | 0 |
| io 563 | 0.9% Normal Saline 300.0 ml + 1200 mgmin Labetolol | 0 |
| io 580 | Cath Lab Output | 0 |
| io 591 | Normal Saline _GU | 0 |
| io 615 | D5/.45NS 2000.0 ml | 0 |
| io 648 | Drain Out #3 Other | 0 |
| io 659 | Other Blood Products | 0 |
| io 703 | Drain Out #1 JP Lateral | 0 |
| io 715 | Urine Out Suprapubic | 0 |
| io 761 | D5W 250.0 ml + 1.5 mcgkgmin Natrecor | 0 |
| io 781 | 0.45% Normal Saline 2000.0 ml | 0 |
| io 898 | D5/.45NS 999.0 ml | 0 |
| io 900 | 0.9% Normal Saline 200.0 ml + 600 mgmin Labetolol | 0 |
| io 926 | Drain Out #1 JP Medial | 0 |
| io 1101 | Protonix | 0 |
| io 1683 | Drain Out #2 JP Lateral | 0 |
| io 1698 | Drain Out #2 Hemovac | 0 |
| io 1707 | Chest Tubes CTICU CT 2 | 0 |
| io 1867 | Drain Out #1 Lumbar | 0 |
| io 1883 | 0.9% Normal Saline 250.0 ml + 25 mcgkgmin Nicardipine | 0 |
| io 1898 | Drain Out #3 T Tube | 0 |
| io 3680 | ProBalance | 0 |
| io 3692 | 0.9% Normal Saline 250.0 ml + 125 mcgkgmin Nicardipine | 0 |
| io 4691 | D5W 250.0 ml + 100Uhr Vasopressin + 100Umin Vasopressin | 0 |
| io 4692 | D5W 250.0 ml + 200Uhr Vasopressin + 200Umin Vasopressin | 0 |
| med 25 | Heparin | 0 |
| med 47 | Levophed | 0 |
| med 49 | Nitroglycerine | 0 |
| med 115 | Diltiazem | 0 |
| med 118 | Fentanyl | 0 |
| med 120 | Levophed-k | 0 |
| med 123 | Lasix | 0 |
| med 126 | Morphine Sulfate | 0 |
| med 127 | Neosynephrine | 0 |
| med 133 | Sandostatin | 0 |
| med 134 | Reopro | 0 |
| med 163 | Dilaudid | 0 |
| totalbal 3 | Blood Products Total | 0 |
| totalbal 4 | Cerebral Drain Total | 0 |
| totalbal 5 | Chest Tube Out Total | 0 |
| totalbal 6 | Colloids Total | 0 |
| totalbal 7 | Drain #1 Total Out | 0 |
| totalbal 8 | Drain #2 Total Out | 0 |
| totalbal 9 | Drain #3 Total Out | 0 |
| totalbal 10 | Drain #4 Total Out | 0 |
| totalbal 24 | Tube Feeds In Total | 0 |
| totalbal 25 | UltrafiltrateTotal | 0 |
| totalbal 27 | 24 h Net Body Balance | 0 |
| totalbal 28 | LOS Net Body Balance | -0.010032666 |

From the 48-hour dataset, 32 features in total were selected (20 primaries and 12 secondaries; some of the primary features had no associated secondary features). Each primary feature and its associated secondary features (if any) made up a “feature group,” giving 20 initial feature groups. An additional 16 features total were added to this feature set to optimize performance on the 6-, 12-, and 24-hour datasets. For example, feature groups that were selected for a different onset period can be selected. These additional features made up a 21^{st} feature group, called “additional” features. For example, in an example embodiment, the system applies the feature selection method described above based on data associated with positives and negatives of developing a disease within each of a plurality of time frames, to identify respective relevant subsets of features for predicting onset of the disease in the respective time frame. This may result in identification of a feature subset as relevant for predicting onset within a first of the time frames, which feature subset had not been identified as relevant for predicting onset within one or more others of the time frames. Nevertheless, in an example embodiment, even if a feature subset had not been selected for prediction of onset within a particular time frame, if the feature subset had been selected for a different time frame, it is used for training a prediction model even for the time frame for which it had not been selected. (If it is subsequently determined that the generated model does not yield satisfactory prediction results for the time frame, then it is discarded as it relates to that time frame.)

Table 4 shows the selected features organized by feature group, including their identifier in the MIMIC II database, the role they play (as primary, secondary, or additional features), and a brief description. Using a separate database, the same procedure as detailed above could be used to identify and select primary and secondary features and additional features from that separate database, using the above methods of the invention, which is within the scope of the invention. Likewise, such separate measurements are within the meaning of the term “feature” as used in this application. Further, as explained above, if a given hospital measures a feature in different units or uses a different type of measurement for the same feature as compared to the MIMIC II database, those data are also “features” as defined herein and can be used in the above selection and prediction methods of the invention, which is within the scope of the invention. As used herein, a “MIMIC II feature” is a feature (whether primary, secondary, additional or remaining) from the MIMIC II database, while a “feature” includes such MIMIC II features and other features that may be identified and/or selected from other hospital databases, in accordance with the invention and as described herein. Such features from other databases are also termed primary, secondary, additional and remaining in accordance with the methods of the invention.

TABLE 4

Feature groups selected in this work. Each row of the table indicates a different feature. The first column lists the feature group by number to which the feature is associated. The second column lists the feature by it identifier in the MIMIC II database and how it was selected (as a 48-hour primary or secondary feature, or as an additional feature). The third column gives a brief description of what the feature measures, which has a well-known meaning to a person of ordinary skill in the art

| Feature Group | Feature Identifier (role) | Brief Feature Description |
|---|---|---|
| 1 | chart 818 (1^{st} primary) | Lactic Acid (0.5-2.0) |
| 1 | chart 1531 (secondary to 1^{st} primary) | Lactic Acid |
| 2 | chart 781 (2^{nd} primary) | BUN (6-20) |
| 2 | chart 1162 (secondary to 2^{nd} primary) | BUN; Blood Urea Nitrogen |
| 3 | chart 828 (3^{rd} primary) | Platelets |
| 3 | chart 811(secondary to 3^{rd} primary) | Glucose (70-105) |
| 3 | chart 1529 (secondary to 3^{rd} primary) | Glucose |
| 4 | totalbal 20 (4^{th} primary) | PO/Gastric In Total |
| 4 | io 102 (secondary to 4^{th} primary) | Po Intake |
| 5 | lab 50019 (5^{th} primary) | PO2 |
| 5 | chart 779 (secondary to 5^{th} primary) | Arterial PaO2 |
| 6 | totalbal 26 (6^{th} primary) | Urine Out Total |
| 6 | totalbal 2 (secondary to 6^{th} primary) | 24-hr Total Out |
| 6 | totalbal 18 (secondary to 6^{th} primary) | IV Infusion In Total |
| 6 | io 55 (secondary to 6^{th} primary) | Urine Out Foley |
| 7 | totalbal 19 (7^{th} primary) | IV Nutrition Total |
| 8 | chart 682 (8^{th} primary) | Tidal Volume (Observ.) Lung Vol. Displac. |
| 9 | chart 785 (9^{th} primary) | CPK/MB Blood Test |
| 10 | io 97 (10^{th} primary) | Cerebral Drain L Ventricular Drain |
| 11 | lab 50017 (11^{th} primary) | PEEP; positive end respiratory pressure |
| 12 | totalbal 1 (12^{th} primary) | 24-hr Total In |
| 13 | totalbal 16 (13^{th} primary) | Gastric Out Total |
| 14 | io 133 (14^{th} primary) | D5W 250.0 ml + 100 mcg/kg/min Nitroglycerine-k |
| 15 | chart 683 (15^{th} primary) | Tidal Volume (Set) |
| 16 | chart 789 (16^{th} primary) | Cholesterol (< 200) |
| 17 | chart 807 (17^{th} primary) | Fingerstick Glucose |
| 18 | chart 815 (18^{th} primary) | INR (2-4 ref. range) |
| 18 | chart 821 (secondary to 18^{th} primary) | Magnesium (1.6-2.6) |
| 18 | chart 1532 (secondary to 18^{th} primary) | Magnesium |
| 18 | lab 50030 (secondary to 18^{th} primary) | free Ca |
| 19 | io 134 (19^{th} primary) | 0.9% Normal Saline 1000 ml |
| 20 | totalbal 23 (20^{th} primary) | Total Hourly Output |
| 21 | chart 1528 (additional) | Fibrinogen |
| 21 | chart 198 (additional) | GCS Total Glasgow Coma Scale |
| 21 | chart 20001 (additional) | SAPS-I Simplified Acute Physiology Score |
| 21 | chart 20009 (additional) | Overall SOFA (Sequen. Organ Failure) Score |
| 21 | chart 211 (additional) | Heart Rate |
| 21 | chart 671 (additional) | TCPCV Insp. Time Ventilation |
| 21 | chart 773 (additional) | Alk. Phosphate |
| 21 | chart 793 (additional) | D-Dimer (0-500) |
| 21 | chart 809 (additional) | Gentamycin/Random |
| 21 | chart 826 (additional) | Phenobarbital |
| 21 | chart 856 (additional) | Vancomycin/Trough |
| 21 | chart 87 (additional) | Braden Score |
| 21 | io 53 (additional) | Stool Out Fecal Bag |
| 21 | io 69 (additional) | Urine Out Void |
| 21 | med 163 (additional) | Dilaudid |
| 21 | totalbal 25 (additional) | UltrafiltrateTotal |

### Example 2: Different Combinations of Patients Produce Models With Similarly High Predictive Accuracy

Because the features within the first 20 feature groups are correlated with each other (especially within feature groups with more than one feature), the inventors carried out a further set of experiments in which we chose two features from each of the first 14 feature sets (but only one feature from feature sets that had only one feature) and two features from the additional set, and tested their predictive ability. Ten independent experiments of this type were carried out using the same features used in the model, but different random divisions of the data into training and testing data. Machine learning as above on the training sets was used to create a model that was then tested on the testing set (containing the patients the model had not seen). The scores on each of ten the testing set are reported in Table 5 for each of the four time points, together with the features in that dataset and the predictive model resulting from the training that produced these results. The results show all of the models have very good predictive capabilities, even though each of the respective models may differ from one another. This is consistent with the features being powerfully useful for accurate prediction.

TABLE 5

The parameters for forty different predictive models are given (ten different sets of parameters each used for the four different time points) together with the performance of those models. Each of the ten parameter sets appears in its own section of the table, followed by the predictive performance of those model parameters. In each set of parameters, the first parameter given is the bias, followed by the coefficient for each of the features from MIMIC II, for the four different models at four different time points in advance of the onset of SIRS in the positive set. Positive coefficients indicate a tendency of the respective parameter predicting being tested positive for SIRS and negative coefficients indicate a tendency of the respective parameter predicting being tested negative for SIRS

| | 48-hour model | 24-hour model | 12-hour model | 6-hour model |
|---|---|---|---|---|
| Set 1 Parameters | | | | |
| bias | 0.615920 | 0.913224 | 1.045310 | 1.196787 |
| chart 1162 | -0.079121 | -0.057250 | 0.000000 | 0.000000 |
| chart 1528 | 0.014627 | -0.011422 | -0.010567 | 0.028798 |
| chart 1531 | -0.079470 | -0.117308 | -0.119365 | -0.081314 |
| chart 198 | 0.000000 | -0.623604 | -0.602151 | -0.434176 |
| chart 682 | 0.129793 | 0.172320 | 0.145261 | 0.000000 |
| chart 779 | 0.059285 | 0.000000 | 0.149554 | 0.276046 |
| chart 781 | 0.083009 | 0.110677 | 0.035790 | 0.000000 |
| chart 785 | -0.051666 | 0.043012 | 0.000000 | 0.000000 |
| chart 811 | -0.583528 | -0.265289 | -0.314617 | -0.173928 |
| chart 818 | -0.048873 | 0.000000 | 0.000000 | 0.000000 |
| chart 828 | -0.633149 | -0.374788 | 0.000000 | -0.228734 |
| io 102 | 0.115260 | 0.108089 | 0.066090 | 0.061826 |
| io 133 | 0.078115 | 0.073909 | 0.093805 | 0.000000 |
| io 97 | 0.013388 | -0.002194 | 0.011098 | 0.063874 |
| lab 50017 | 0.061014 | 0.000000 | 0.000000 | 0.000000 |
| lab 50019 | -0.159398 | -0.073326 | -0.024099 | 0.121263 |
| totalbal 1 | -0.119435 | 0.015848 | 0.055107 | 0.079042 |
| totalbal 16 | 0.014986 | -0.020905 | -0.044861 | -0.046192 |
| totalbal 19 | 0.019590 | 0.033700 | 0.013719 | 0.008559 |
| totalbal 2 | -0.116085 | 0.000000 | -0.058382 | -0.036658 |
| totalbal 20 | -0.340788 | -0.307695 | -0.382889 | -0.424959 |
| totalbal 26 | -0.405579 | -0.358916 | -0.317020 | -0.327842 |

Set 1 Predictive Results

| Accuracy | 83.95% | 81.47% | 81.03% | 81.15% |
|---|---|---|---|---|
| PPV | 87.71% | 86.66% | 84.71% | 84.16% |
| Sensitivity | 86.65% | 86.10% | 89.75% | 92.34% |
| Specificity | 79.35% | 71.54% | 58.93% | 47.01% |
| NPV | 77.76% | 70.56% | 69.40% | 66.82% |
| AUC | 83.00% | 78.82% | 74.34% | 69.68% |
| Set 2 Parameters | | | | |
| bias | 0.59695573 | 0.909521 | 1.057665 | 1.229083 |
| chart 1162 | 0.082354 | 0.034969 | 0.000000 | 0.000000 |
| chart 1529 | -0.384999 | -0.174643 | -0.171213 | -0.074010 |
| chart 1531 | -0.031844 | -0.103458 | -0.090403 | -0.040508 |
| chart 198 | 0.000000 | -0.605356 | -0.586906 | -0.396961 |
| chart 20001 | 0.000000 | -0.288058 | -0.473570 | -0.439801 |
| chart 682 | 0.115831 | 0.216234 | 0.227728 | 0.000000 |
| chart 779 | 0.029516 | 0.000000 | 0.138712 | 0.312833 |
| chart 781 | -0.087739 | 0.051992 | 0.071431 | 0.000000 |
| chart 785 | -0.066235 | 0.025113 | 0.000000 | 0.000000 |
| chart 818 | -0.084714 | 0.000000 | 0.000000 | 0.000000 |
| chart 828 | -0.725970 | -0.381094 | 0.000000 | -0.220024 |
| io 102 | 0.104973 | 0.112235 | 0.076689 | 0.071534 |
| io 133 | 0.074103 | 0.086076 | 0.112418 | 0.000000 |
| io 97 | 0.019507 | -0.003578 | 0.010001 | 0.058538 |
| lab 50017 | 0.064066 | 0.000000 | 0.000000 | 0.000000 |
| lab 50019 | -0.178200 | -0.080493 | -0.034266 | 0.133420 |
| totalbal 1 | -0.096806 | 0.066154 | 0.110757 | 0.171955 |
| totalbal 16 | 0.029085 | -0.001094 | -0.010024 | -0.001588 |
| totalbal 18 | -0.171826 | -0.054175 | -0.047516 | -0.048636 |
| totalbal 19 | 0.018322 | 0.039806 | 0.023564 | 0.016940 |
| totalbal 20 | -0.354524 | -0.278917 | -0.325748 | -0.364174 |
| totalbal 26 | -0.480544 | -0.285616 | -0.254159 | -0.245701 |
| Set 2 Predictive Results | | | | |
| Accuracy | 83.61% | 82.11% | 81.86% | 82.09% |
| PPV | 87.10% | 86.94% | 86.17% | 86.25% |
| Sensitivity | 86.83% | 86.82% | 88.99% | 90.67% |
| Specificity | 78.14% | 72.00% | 63.81% | 55.94% |
| NPV | 77.73% | 71.78% | 69.56% | 66.29% |
| AUC | 82.49% | 79.41% | 76.40% | 73.30% |
| Set 3 Parameters | | | | |
| bias | 0.61676526 | 0.869603 | 1.016045 | 1.201040 |
| chart 1162 | -0.074871 | -0.041301 | 0.000000 | 0.000000 |
| chart 1531 | -0.079106 | -0.040634 | -0.043314 | -0.017709 |
| chart 20001 | 0.000000 | -0.228548 | -0.418474 | -0.399840 |
| chart 20009 | 0.000000 | -0.296936 | -0.272117 | -0.158803 |
| chart 682 | 0.105667 | 0.299904 | 0.319713 | 0.000000 |
| chart 779 | 0.047056 | 0.000000 | 0.186297 | 0.359814 |
| chart 781 | 0.068296 | 0.138811 | 0.098510 | 0.000000 |
| chart 785 | -0.046401 | 0.018356 | 0.000000 | 0.000000 |
| chart 811 | -0.573524 | -0.354420 | -0.467158 | -0.217760 |
| chart 818 | -0.052816 | 0.000000 | 0.000000 | 0.000000 |
| chart 828 | -0.630249 | -0.520801 | 0.000000 | -0.305409 |
| io 102 | 0.042085 | 0.021683 | -0.028267 | -0.023195 |
| io 133 | 0.065330 | 0.071635 | 0.111925 | 0.000000 |
| io 55 | 0.258612 | 0.279953 | 0.234635 | 0.248006 |
| io 97 | 0.013375 | -0.002941 | 0.016216 | 0.049905 |
| lab 50017 | 0.050744 | 0.000000 | 0.000000 | 0.000000 |
| lab 50019 | -0.158154 | -0.037681 | -0.001000 | 0.169138 |
| totalbal 1 | -0.163007 | 0.033040 | 0.084071 | 0.149264 |
| totalbal 16 | -0.000048 | 0.012147 | -0.010687 | -0.002006 |
| totalbal 19 | 0.009974 | 0.041029 | 0.025875 | 0.009955 |
| totalbal 20 | -0.306671 | -0.325202 | -0.361284 | -0.372054 |
| totalbal 26 | -0.595502 | -0.578033 | -0.511842 | -0.455543 |
| Set 3 Predictive Results | | | | |
| Accuracy | 83.87% | 82.50% | 79.98% | 80.83% |
| PPV | 87.76% | 86.75% | 84.20% | 84.92% |
| Sensitivity | 86.43% | 87.75% | 88.74% | 90.64% |
| Specificity | 79.50% | 71.23% | 57.77% | 50.92% |
| NPV | 77.51% | 73.04% | 66.94% | 64.09% |
| AUC | 82.97% | 79.49% | 73.26% | 70.78% |
| Set 4 Parameters | | | | |
| bias | 0.59725842 | 0.857635 | 1.027098 | 1.188486 |
| chart 1162 | 0.088805 | 0.077187 | 0.000000 | 0.000000 |
| chart 1529 | -0.388303 | -0.253960 | -0.242489 | -0.122255 |
| chart 1531 | -0.031446 | -0.011477 | -0.016946 | -0.006168 |
| chart 20009 | 0.000000 | -0.406286 | -0.454881 | -0.366385 |
| chart 211 | 0.000000 | 0.000000 | -0.477251 | -0.113969 |
| chart 682 | 0.116333 | 0.308925 | 0.390253 | 0.000000 |
| chart 779 | 0.032301 | 0.000000 | 0.134304 | 0.353429 |
| chart 781 | -0.093651 | 0.026673 | 0.087839 | 0.000000 |
| chart 785 | -0.065092 | -0.002758 | 0.000000 | 0.000000 |
| chart 818 | -0.090451 | 0.000000 | 0.000000 | 0.000000 |
| chart 828 | -0.739739 | -0.617315 | 0.000000 | -0.362596 |
| io 102 | 0.119758 | 0.082825 | 0.038018 | 0.040678 |
| io 133 | 0.075430 | 0.075379 | 0.092312 | 0.000000 |
| io 97 | 0.017009 | 0.012403 | 0.023001 | 0.065841 |
| lab 50017 | 0.061409 | 0.000000 | 0.000000 | 0.000000 |
| lab 50019 | -0.179407 | -0.064676 | -0.024317 | 0.154678 |
| totalbal 1 | -0.150346 | 0.001785 | 0.122049 | 0.148969 |
| totalbal 16 | 0.021809 | 0.022513 | -0.006013 | 0.006992 |
| totalbal 19 | 0.020089 | 0.052639 | 0.029443 | 0.023396 |
| totalbal 2 | -0.135348 | 0.000000 | -0.074924 | -0.033416 |
| totalbal 20 | -0.354069 | -0.393439 | -0.407032 | -0.457152 |
| totalbal 26 | -0.438391 | -0.510071 | -0.373762 | -0.377456 |
| Set 4 Predictive Results | | | | |
| Accuracy | 83.47% | 81.84% | 80.00% | 80.18% |
| PPV | 86.94% | 85.65% | 83.87% | 83.97% |
| Sensitivity | 86.79% | 88.15% | 89.29% | 91.06% |
| Specificity | 77.84% | 68.29% | 56.46% | 47.01% |
| NPV | 77.60% | 72.85% | 67.53% | 63.30% |
| AUC | 82.31% | 78.22% | 72.88% | 69.04% |
| Set 5 Parameters | | | | |
| bias | 0.61450305 | 0.859746 | 1.014454 | 1.162259 |
| chart 1162 | -0.074703 | -0.058870 | 0.000000 | 0.000000 |
| chart 1531 | -0.076971 | -0.053235 | -0.087837 | -0.038245 |
| chart 211 | 0.000000 | 0.000000 | -0.410562 | -0.107260 |
| chart 671 | 0.102520 | 0.082216 | 0.074346 | 0.090695 |
| chart 682 | 0.122927 | 0.208807 | 0.249759 | 0.000000 |
| chart 779 | 0.062050 | 0.000000 | 0.170336 | 0.326798 |
| chart 781 | 0.076803 | 0.042078 | 0.005781 | 0.000000 |
| chart 785 | -0.050517 | 0.024896 | 0.000000 | 0.000000 |
| chart 811 | -0.573466 | -0.389515 | -0.369917 | -0.252222 |
| chart 818 | -0.046721 | 0.000000 | 0.000000 | 0.000000 |
| chart 828 | -0.626180 | -0.507826 | 0.000000 | -0.302875 |
| io 102 | 0.102338 | 0.074418 | 0.027698 | 0.026270 |
| io 133 | 0.077823 | 0.071404 | 0.084093 | 0.000000 |
| io 97 | 0.014070 | 0.013240 | 0.022705 | 0.071831 |
| lab 50017 | 0.048191 | 0.000000 | 0.000000 | 0.000000 |
| lab 50019 | -0.154367 | -0.048937 | -0.008529 | 0.153081 |
| totalbal 1 | -0.071973 | 0.035419 | 0.109590 | 0.149382 |
| totalbal 16 | 0.024412 | -0.001314 | -0.039416 | -0.030252 |
| totalbal 18 | -0.144695 | -0.161657 | -0.157742 | -0.174891 |
| totalbal 19 | 0.018760 | 0.035403 | 0.013320 | 0.009398 |
| totalbal 20 | -0.341943 | -0.414468 | -0.444812 | -0.488835 |
| totalbal 26 | -0.450586 | -0.493292 | -0.391937 | -0.382170 |
| Set 5 Predictive Results | | | | |
| Accuracy | 84.09% | 80.81% | 79.74% | 80.36% |
| PPV | 87.91% | 84.65% | 83.05% | 83.35% |
| Sensitivity | 86.65% | 87.79% | 90.15% | 92.37% |
| Specificity | 79.73% | 65.82% | 53.36% | 43.75% |
| NPV | 77.84% | 71.51% | 68.11% | 65.28% |
| AUC | 83.19% | 76.80% | 71.76% | 68.06% |
| Set 6 Parameters | | | | |
| bias | 0.593348 | 0.844009 | 0.958029 | 1.141223 |
| chart 1162 | 0.085433 | 0.066645 | 0.000000 | 0.000000 |
| chart 1529 | -0.383465 | -0.256067 | -0.316012 | -0.165695 |
| chart 1531 | -0.032675 | -0.053443 | -0.024053 | -0.029464 |
| chart 671 | 0.101643 | 0.071712 | 0.067672 | 0.082138 |
| chart 682 | 0.082330 | 0.156451 | 0.156465 | 0.000000 |
| chart 773 | 0.039828 | 0.000000 | 0.000000 | 0.000000 |
| chart 779 | 0.023593 | 0.000000 | 0.116854 | 0.262436 |
| chart 781 | -0.108960 | -0.093785 | -0.136490 | 0.000000 |
| chart 785 | -0.061386 | 0.013126 | 0.000000 | 0.000000 |
| chart 818 | -0.098447 | 0.000000 | 0.000000 | 0.000000 |
| chart 828 | -0.740965 | -0.592884 | 0.000000 | -0.392499 |
| io 102 | 0.041260 | 0.025058 | -0.009344 | -0.016451 |
| io 133 | 0.063383 | 0.059303 | 0.076644 | 0.000000 |
| io 55 | 0.266610 | 0.236793 | 0.166960 | 0.162403 |
| io 97 | 0.011900 | 0.004938 | 0.031009 | 0.061142 |
| lab 50017 | 0.039830 | 0.000000 | 0.000000 | 0.000000 |
| lab 50019 | -0.172980 | -0.079832 | -0.030026 | 0.141330 |
| totalbal 1 | -0.196710 | -0.073132 | -0.042887 | 0.021993 |
| totalbal 16 | 0.005866 | -0.024535 | -0.051521 | -0.055779 |
| totalbal 19 | 0.007636 | 0.024136 | 0.007213 | -0.005093 |
| totalbal 20 | -0.323203 | -0.398548 | -0.489053 | -0.477262 |
| totalbal 26 | -0.650986 | -0.662500 | -0.659062 | -0.555817 |
| Set 6 Predictive Results | | | | |
| Accuracy | 83.67% | 80.71% | 79.06% | 80.30% |
| PPV | 87.25% | 84.24% | 81.15% | 83.06% |
| Sensitivity | 86.74% | 88.22% | 92.22% | 92.76% |
| Specificity | 78.44% | 64.58% | 45.71% | 42.31% |
| NPV | 77.68% | 71.86% | 69.86% | 65.72% |
| AUC | 82.59% | 76.40% | 68.96% | 67.54% |
| Set 7 Parameters | | | | |
| bias | 0.613974 | 0.858309 | 0.974021 | 1.150347 |
| chart 1162 | -0.077308 | -0.060797 | 0.000000 | 0.000000 |
| chart 1531 | -0.080661 | -0.054176 | -0.057023 | -0.038605 |
| chart 682 | 0.131826 | 0.206607 | 0.185004 | 0.000000 |
| chart 773 | 0.048919 | 0.000000 | 0.000000 | 0.000000 |
| chart 779 | 0.062932 | 0.000000 | 0.182390 | 0.320940 |
| chart 781 | 0.073781 | 0.044840 | -0.046120 | 0.000000 |
| chart 785 | -0.051381 | 0.027303 | 0.000000 | 0.000000 |
| chart 793 | -0.009142 | 0.004290 | -0.020299 | -0.003593 |
| chart 811 | -0.588521 | -0.395632 | -0.506384 | -0.285913 |
| chart 818 | -0.052222 | 0.000000 | 0.000000 | 0.000000 |
| chart 828 | -0.639125 | -0.517775 | 0.000000 | -0.332109 |
| io 102 | 0.113349 | 0.082236 | 0.039937 | 0.039174 |
| io 133 | 0.078293 | 0.072124 | 0.086527 | 0.000000 |
| io 97 | 0.013171 | 0.012923 | 0.027322 | 0.070743 |
| lab 50017 | 0.060678 | 0.000000 | 0.000000 | 0.000000 |
| lab 50019 | -0.157873 | -0.051702 | -0.005444 | 0.154763 |
| totalbal 1 | -0.118569 | -0.047450 | 0.008415 | 0.051431 |
| totalbal 16 | 0.012901 | -0.016717 | -0.043213 | -0.043930 |
| totalbal 19 | 0.017418 | 0.034014 | 0.015434 | 0.009239 |
| totalbal 2 | -0.113743 | 0.000000 | -0.105284 | -0.054249 |
| totalbal 20 | -0.339247 | -0.412832 | -0.498335 | -0.503869 |
| totalbal 26 | -0.403838 | -0.536333 | -0.453441 | -0.408015 |
| Set 7 Predictive Results | | | | |
| Accuracy | 83.98% | 81.20% | 79.09% | 80.14% |
| PPV | 87.78% | 84.80% | 81.70% | 83.01% |
| Sensitivity | 86.61% | 88.26% | 91.28% | 92.58% |
| Specificity | 79.50% | 66.05% | 48.18% | 42.23% |
| NPV | 77.74% | 72.37% | 68.54% | 65.11% |
| AUC | 83.06% | 77.15% | 69.73% | 67.40% |
| Set 8 Parameters | | | | |
| bias | 0.596690 | 0.845004 | 0.962103 | 1.147989 |
| chart 1162 | 0.081725 | 0.061713 | 0.000000 | 0.000000 |
| chart 1529 | -0.385030 | -0.256333 | -0.312654 | -0.165197 |
| chart 1531 | -0.031393 | -0.036796 | -0.019196 | -0.019240 |
| chart 682 | 0.115715 | 0.197238 | 0.187996 | 0.000000 |
| chart 779 | 0.029759 | 0.000000 | 0.126490 | 0.284521 |
| chart 781 | -0.086768 | -0.085388 | -0.123277 | 0.000000 |
| chart 785 | -0.065524 | 0.009114 | 0.000000 | 0.000000 |
| chart 793 | -0.006505 | 0.000326 | -0.014960 | -0.003230 |
| chart 809 | -0.030555 | 0.000000 | 0.000000 | 0.000000 |
| chart 818 | -0.084226 | 0.000000 | 0.000000 | 0.000000 |
| chart 828 | -0.725962 | -0.578702 | 0.000000 | -0.384182 |
| io 102 | 0.105130 | 0.073824 | 0.031866 | 0.026925 |
| io 133 | 0.074125 | 0.068055 | 0.077929 | 0.000000 |
| io 97 | 0.020052 | 0.014244 | 0.031479 | 0.073195 |
| lab 50017 | 0.064934 | 0.000000 | 0.000000 | 0.000000 |
| lab 50019 | -0.178624 | -0.080260 | -0.033336 | 0.142417 |
| totalbal 1 | -0.096818 | 0.018763 | 0.064525 | 0.128774 |
| totalbal 16 | 0.029274 | 0.002096 | -0.021955 | -0.027764 |
| totalbal 18 | -0.171310 | -0.173592 | -0.209381 | -0.187955 |
| totalbal 19 | 0.018210 | 0.034869 | 0.014609 | 0.007114 |
| totalbal 20 | -0.354150 | -0.419509 | -0.518971 | -0.508489 |
| totalbal 26 | -0.480792 | -0.506810 | -0.514822 | -0.411002 |
| Set 8 Predictive Results | | | | |
| Accuracy | 83.59% | 80.54% | 78.69% | 80.28% |
| PPV | 87.07% | 84.33% | 81.27% | 83.11% |
| Sensitivity | 86.83% | 87.79% | 91.34% | 92.63% |
| Specificity | 78.06% | 64.97% | 46.64% | 42.63% |
| NPV | 77.71% | 71.25% | 67.98% | 65.48% |
| AUC | 82.45% | 76.38% | 68.99% | 67.63% |
| Set 9 Parameters | | | | |
| bias | 0.616284 | 0.856007 | 0.973617 | 1.146810 |
| chart 1162 | -0.075573 | -0.052118 | 0.000000 | 0.000000 |
| chart 1531 | -0.078892 | -0.061977 | -0.064236 | -0.046893 |
| chart 682 | 0.105076 | 0.175308 | 0.162112 | 0.000000 |
| chart 779 | 0.047354 | 0.000000 | 0.172271 | 0.302153 |
| chart 781 | 0.069215 | 0.037245 | -0.050473 | 0.000000 |
| chart 785 | -0.045371 | 0.032464 | 0.000000 | 0.000000 |
| chart 809 | -0.021142 | 0.000000 | 0.000000 | 0.000000 |
| chart 811 | -0.572875 | -0.394216 | -0.505004 | -0.283510 |
| chart 818 | -0.052810 | 0.000000 | 0.000000 | 0.000000 |
| chart 826 | 0.025027 | 0.030628 | 0.023386 | 0.010586 |
| chart 828 | -0.630778 | -0.520479 | 0.000000 | -0.330871 |
| io 102 | 0.042961 | 0.022472 | -0.017407 | -0.017328 |
| io 133 | 0.065725 | 0.061135 | 0.082630 | 0.000000 |
| io 55 | 0.256709 | 0.239647 | 0.168766 | 0.165857 |
| io 97 | 0.013305 | 0.001418 | 0.025208 | 0.060620 |
| lab 50017 | 0.050653 | 0.000000 | 0.000000 | 0.000000 |
| lab 50019 | -0.158086 | -0.052493 | -0.005761 | 0.150331 |
| totalbal 1 | -0.163953 | -0.054532 | -0.020873 | 0.034326 |
| totalbal 16 | 0.001116 | -0.027638 | -0.055034 | -0.056320 |
| totalbal 19 | 0.010041 | 0.023816 | 0.007403 | -0.003018 |
| totalbal 20 | -0.307291 | -0.391029 | -0.467305 | -0.469405 |
| totalbal 26 | -0.595705 | -0.639719 | -0.604781 | -0.536831 |
| Set 9 Predictive Results | | | | |
| Accuracy | 83.92% | 81.22% | 79.17% | 80.30% |
| PPV | 87.80% | 84.79% | 81.65% | 83.13% |
| Sensitivity | 86.48% | 88.33% | 91.52% | 92.63% |
| Specificity | 79.58% | 65.97% | 47.87% | 42.71% |
| NPV | 77.58% | 72.47% | 69.01% | 65.53% |
| AUC | 83.03% | 77.15% | 69.70% | 67.67% |
| Set 10 Parameters | | | | |
| bias | 0.6150488 | 0.856018 | 0.973612 | 1.148259 |
| chart 1162 | -0.075004 | -0.058842 | 0.000000 | 0.000000 |
| chart 1531 | -0.077247 | -0.059779 | -0.065061 | -0.041495 |
| chart 682 | 0.130835 | 0.199684 | 0.176202 | 0.000000 |
| chart 779 | 0.060598 | 0.000000 | 0.180059 | 0.315992 |
| chart 781 | 0.080529 | 0.041449 | -0.053515 | 0.000000 |
| chart 785 | -0.050856 | 0.027466 | 0.000000 | 0.000000 |
| chart 811 | -0.583981 | -0.391430 | -0.499748 | -0.283068 |
| chart 818 | -0.047807 | 0.000000 | 0.000000 | 0.000000 |
| chart 828 | -0.632393 | -0.512623 | 0.000000 | -0.327204 |
| io 102 | 0.105973 | 0.125834 | 0.066088 | 0.052974 |
| io 133 | 0.078028 | 0.068592 | 0.086727 | 0.000000 |
| io 53 | 0.046452 | 0.000000 | 0.040153 | 0.028834 |
| io 69 | 0.000000 | -0.145370 | -0.109129 | -0.056514 |
| io 97 | 0.012542 | 0.028264 | 0.039309 | 0.071315 |
| lab 50017 | 0.062166 | 0.000000 | 0.000000 | 0.000000 |
| lab 50019 | -0.158595 | -0.061223 | -0.010076 | 0.149672 |
| totalbal 1 | -0.119621 | -0.051519 | 0.005576 | 0.047070 |
| totalbal 16 | 0.014249 | -0.018354 | -0.045066 | -0.044854 |
| totalbal 19 | 0.018959 | 0.035018 | 0.013853 | 0.005953 |
| totalbal 2 | -0.116124 | 0.000000 | -0.106958 | -0.052790 |
| totalbal 20 | -0.339871 | -0.405354 | -0.491820 | -0.502403 |
| totalbal 26 | -0.402412 | -0.515802 | -0.430860 | -0.399246 |
| Set 10 Predictive Results | | | | |
| Accuracy | 84.01% | 80.98% | 78.98% | 80.30% |
| PPV | 87.79% | 84.56% | 81.56% | 83.09% |
| Sensitivity | 86.65% | 88.22% | 91.34% | 92.71% |
| Specificity | 79.50% | 65.43% | 47.64% | 42.47% |
| NPV | 77.79% | 72.12% | 68.44% | 65.64% |
| AUC | 83.08% | 76.82% | 69.49% | 67.59% |

As shown in the below examples, depending on the number of features identified using the above methods of the invention, the accuracy of predicting whether or not SIRS will occur can be predicted at 60% or greater, more preferably 70% or greater and most preferably 80% or greater. Predictions of patients likely to develop SIRS can lead to improved healthcare outcomes and reduced cost by appropriate monitoring and intervention.

### Example 3: Models With Five Features Show a Range of Predictive Abilities

Machine learning was applied to the MIMIC II database as described above, using logistic regression on the 48-hour dataset, using feature sets of five features selected from the first 20 groups of Table 4. Machine learning models developed on a training dataset produced a wide range of accuracies when applied to a testing dataset, from above 80% to below 70%, depending on the particular feature set used in the learning, as shown in Table 6.

TABLE 6

The parameters for 4 different predictive models trained and tested with the 48-hour dataset from MIMIC II are given together with the performance of those models
48-hour model
Set 1 Parameters

| bias | 0.60467938 |
|---|---|
| chart 811 | -0.806772715 |
| chart 818 | -0.006220046 |
| chart 1532 | -0.401452681 |
| lab 50030 | -0.044147911 |
| totalbal 26 | -0.695661357 |
| Set 1 Predictive Results | |
| Accuracy | 83.67% |
| PPV | 87.42% |
| Sensitivity | 86.52% |
| Specificity | 78.82% |
| NPV | 77.47% |
| AUC | 82.67% |
| Set 2 Parameters | |
| bias | 0.490888658 |
| lab 50030 | -0.248727074 |
| io 55 | 0.173756459 |
| io 97 | 0.015815466 |
| totalbal 2 | -1.360023384 |
| totalbal 16 | 0.035450753 |
| Set 2 Predictive Results | |
| Accuracy | 78.77% |
| PPV | 78.52% |
| Sensitivity | 91.24% |
| Specificity | 57.56% |
| NPV | 79.44% |
| AUC | 74.40% |
| Set 3 Parameters | |
| bias | 0.495140624 |
| chart 818 | -0.158001857 |
| chart 1162 | -0.778137982 |
| lab 50019 | -0.363315949 |
| io 133 | -0.017914369 |
| totalbal 16 | -0.137483415 |
| Set 3 Predictive Results | |
| Accuracy | 72.27% |
| PPV | 72.50% |
| Sensitivity | 90.17% |
| Specificity | 41.83% |
| NPV | 71.45% |
| AUC | 66.00% |
| bias | 0.508688002 |
| chart 682 | -0.123597317 |
| chart 1531 | -0.338819848 |
| lab 50019 | -0.38430722 |
| io 97 | -0.020770998 |
| totalbal 16 | -0.157934522 |
| Accuracy | 68.07% |
| PPV | 68.47% |
| Sensitivity | 91.37% |
| Specificity | 28.44% |
| NPV | 65.96% |
| AUC | 59.91% |

### Example 4: Models With One or Two Features Also Show Predictive Abilities

Machine learning was applied to the MIMIC II database as described above, using logistic regression on the 48-hour dataset, using feature sets of one and two features selected from the first 20 groups of Table 4. Machine learning models developed on the training dataset produced useful accuracies when applied to the testing dataset, as shown in Table 7.

TABLE 7

The parameters for 4 different predictive models trained and tested with the 48-hour dataset from MIMIC II are given together with the performance of those models
48-hour model
Set 1 Parameters (2 features)

| bias | 0.38570712 |
|---|---|
| lab 50019 | -0.353130872 |
| io 102 | -0.565988388 |
| Set 1 Predictive Results | |
| Accuracy | 71.46% |
| PPV | 71.55% |
| Sensitivity | 90.75% |
| Specificity | 38.65% |
| NPV | 71.07% |
| AUC | 64.70% |
| Set 2 Parameters (2 features) | |
| bias | 0.391267846 |
| lab 50017 | -0.136826972 |
| io 97 | -0.023012269 |
| Set 2 Predictive Results | |
| Accuracy | 68.32% |
| PPV | 67.99% |
| Sensitivity | 93.91 % |
| Specificity | 24.81% |
| NPV | 70.54% |
| AUC | 59.36% |
| bias | 0.394150338 |
| lab 50019 | -0.389236239 |
| Accuracy | 66.86% |
| PPV | 67.61% |
| Sensitivity | 90.93% |
| Specificity | 25.95% |
| NPV | 62.71% |
| AUC | 58.44% |
| bias | 0.393311091 |
| chart 682 | -0.304736694 |
| Accuracy | 66.81 % |
| PPV | 66.65% |
| Sensitivity | 94.66% |
| Specificity | 19.44% |
| NPV | 68.17% |
| AUC | 57.05% |

### Example 5: Use of the Invention in a Hospital Setting

Using the invention, the probability of SIRS onset within a given time window for a given patient can be determined. The methods deployed here show methods for building predictive models for which patients will and which will not develop SIRS in a given time frame using a relatively small number of features (patient data measurements) pared down from the much larger number frequently available in a hospital database, such as the MIMIC II database. The models developed and shown here can be used directly to make predictions on hospital patients. One merely needs to acquire measurements of data for a particular patient corresponding to the features in the model, normalize them as shown here, use the model parameters (bias b and coefficients wⱼ), and apply the logistic regression formula to produce a probability of SIRS in the patient at the time point indicated by the model (6, 12, 24, or 48 hours). If the probability is greater than 50% (one-half), then SIRS is predicted; otherwise, it is not. As illustrated above, the probability can be used in a multitude of ways to assign a more fined grained classification of the likelihood of the patient developing SIRS.

The unexpectedly high predictive accuracy for SIRS of the methods of the invention has been shown in this application, for example, by the above accuracy and other determinations in the Predictive Results of Tables 2, 5, 6, and 7. The unexpectedly high predictive accuracy with relatively small sets of feature measurements has also been shown in this application. For example, using the features of Set 1 in Table 6, the method of the invention resulted in an 83.67% value for Accuracy regarding onset of SIRS in a 48-hour model. At its most general terms, this indicates that when the features of that Set 1 were applied to the above model based on the MIMIC II database, the predicted probability (yes or no) of the onset of SIRS at 48 hours resulted in 83.67% Accuracy. In other words, the Set 1 features were applied to the 80% of data designating as training data according to the above method to determine the probability of SIRS onset at 48 hours using those features, and the Accuracy result of 83.67% was determined against the 20% test data relative to those same features and whether or not SIRS occurred at 48 hours, as a person of ordinary skill in the art would appreciate.

Rather than use the precise models presented here directly, one can use the methods here to produce new models, using available hospital data (for example, historical or retrospective data from the previous few weeks, months, or years at the same or similar hospital or hospital system) and apply the methods of the invention to identify feature sets and models, and then to apply them as described here. The methods shown here can be used to prepare the data, select features, and carry out machine learning to produce models and evaluate the predictive ability of those models. The methods shown here can then be used to apply those models to make predictions on new patients using current measurements on those new patients.

For example, with regard to a patient who walks in the door of a hospital for assessment, the invention can be applied in the following manner relative to the MIMIC II database features. The patient’s data can be obtained for the various primary, secondary, and additional features over the course of time and in the ordinary course of the patient’s stay in the hospital. To the extent that the obtained measurements match any of the above models and their Parameter Sets, the method of the invention and the above models can be applied to the patient’s features to determine the probability of the patient developing SIRS at 6, 12, 24 or 48 hours in the future. For example, if one has the measurement corresponding to lab 50019 (Set 3 from Table 7), one can make a prediction using that patient measurement, normalizing, and applying the coefficient and bias from the table to produce a probability of SIRS onset 48-hours into the future from when the measurement was taken. If one has the measurement corresponding to lab 50019 and that corresponding to io 102 (Set 1 from Table 7), then one can make a prediction using those two patient measurements, normalizing, and applying the coefficients and bias from the table to produce a probability of SIRS onset 48-hours into the future from when the measurements were taken. From the results in Table 7, this two-feature model is expected to be more accurate than the one-feature model using only feature 50019 (Accuracy of 71.46% rather than 66.86%). If the model predicts such a probability of the onset of SIRS, the hospital can advantageously begin treating the patient for SIRS or sepsis before the onset of any symptoms, saving time and money as compared to waiting for the more dire situation where SIRS or sepsis symptoms have already occurred.

Alternatively, as features of the patient are ascertained during his or her stay at the hospital, new models can be created based on those features as described above (using the MIMIC II database) and tested for predictive accuracy in terms of the probability of SIRS onset in the patient. That is, if a patient’s measurements correspond to a combination of features for which a model hasn’t previously been trained, one can use methods described here to train such a model using historical (past) data with those features only. One can test those models on historical (past) testing set data as described here. One can assess the accuracy and other metrics quantifying the performance of the model on patients in the testing set as described here. Finally, one can then apply the model to the new patient or to new patients as described here. In this case, as in the others described here, treatment of the patient or patients for SIRS or sepsis can be advantageously initiated before the onset of SIRS or sepsis if the model predicts that it is probable the patient will have SIRS 6, 12, 24, or 48 hours in the future. Alternatively, a hospital could base the decision on whether to begin treatment for SIRS or sepsis in an asymptomatic patient based on the relative Predictive Results of the model (e.g., such treatment would begin in an asymptomatic patient for SIRS that the model of the invention predicts is probable for developing SIRS at a given time if the Predictive Results show an Accuracy of greater than 60% or greater than 70% or greater than 80%, etc.). For example, using a model with accuracy of 60-70% a given hospital may choose to only initiate treatment if the model predicts a 90% or greater probability of developing SIRS, but using a model with accuracy of 70-80% the same hospital may choose to initiate treatment if the model predicts an 80% or greater probability of developing SIRS, and using a model with accuracy of greater than 80% the same hospital may choose to initiate treatment if the model predicts a 70% or greater probability of developing SIRS.

On the other hand, a patient could walk in the door of a hospital that measures features in a manner that is different from that of the MIMIC II database (or some features are the same and one or more features are different in terms of units or a different measurement that is used to assess the same aspect of a patient or a different dose of the same or different medication is used to treat the same aspect of a patient, etc.). First, the features that are different than the MIMIC II features can be mapped to the MIMIC II features by recognizing the similarity of what the measurement achieves (for example, different ways of measuring blood urea [group 2], glucose levels [group 3], cholesterol [group 16], and blood coagulability [chart 815 in group 18]). Then the above models or new models can be used in accordance with the invention to assess the probability of SIRS onset at a given time in the future, with advantageous early treatment being applied as set forth in the above paragraph. For example, simply developing new normalization parameters for new measurements using the method for how normalization was carried out here would allow new measurements to be incorporated into the models presented here. Alternatively, if there is an existing database for the particular hospital that uses features other than MIMIC II features (or a mixture of MIMIC II features and other features), new models can be prepared in accordance with the methods of the invention to select primary, secondary, and additional features from that database that can be used to predict the probability of SIRS onset in a patient in accordance with the methods of the invention described herein. As described here, features would be eliminated and selected, data normalized, and models built and tested using the methods disclosed in this application. The patient’s data then can be obtained for these various primary, secondary, and additional features over the course of time and in the ordinary course of the patient’s stay in the hospital. These new models prepared using the hospital’s database can be applied to the patient’s features to determine the probability of the patient developing SIRS at 6, 12, 24, or 48 hours in the future. Patient measurements can be normalized, inserted into the model, and the model would then make a prediction regarding the probability of the onset of SIRS. Alternatively, as features of the patient are ascertained (measured) during his or her stay at the hospital, new models can be created based on those features in accordance with the methods described above (using the hospital’s database) and tested for predictive accuracy in terms of the probability of SIRS onset in the patient using historical (past) patients at the same or similar hospital or hospital system, as described above. New measurements for the patient can be used in these new models to predict the probability of the onset of SIRS in the new patient. In either case, treatment of the patient for SIRS can be advantageously initiated before the onset of SIRS if the model predicts that it is probable the patient will have SIRS 6, 12, 24, or 48 hours in the future. Alternatively, a hospital could base the decision on whether to begin treatment for SIRS in an asymptomatic patient based on the relative Predictive Results of the model (e.g., such treatment would begin in an asymptomatic patient for SIRS that the model of the invention predicts is probable for developing SIRS at a given time if the Predictive Results show an Accuracy of greater than 60% or greater than 70% or greater than 80%, etc.). For example, using a model with accuracy of 60-70%, a given hospital may choose to only initiate treatment if the model predicts a 90% or greater probability of developing SIRS, but using a model with accuracy of 70-80%, the same hospital may choose to initiate treatment if the model predicts an 80% or greater probability of developing SIRS, and using a model with accuracy of greater than 80%, the same hospital may choose to initiate treatment if the model predicts a 70% or greater probability of developing SIRS.

In another example embodiment of the invention, a hospital, medical center, or health care system maintains multiple models simultaneously. The measurements for a patient can be input into multiple models to obtain multiple probabilities of the onset of SIRS at the same or different times in the future. These different predictive probabilities can be combined to develop an aggregate likelihood or probability of developing SIRS and an action plan can be developed accordingly. For example, the different models could vote as to whether they expected SIRS onset within a given timeframe, and the aggregate prediction could be made based on the outcome of this voting scheme. The voting can be unweighted (each model receives an equal vote), or weighted based on the accuracy or other quantitative metric of the predictive abilities of each model (with more accurate or higher quality models casting a higher proportional vote).

In yet another example embodiment of the invention, one can use multiple models and base a prediction on the first one for which a sufficient number of measurements have been obtained for the current patient. In another aspect of the invention, in any of the embodiments described, the parameters for a model can be re-computed (updated) using additional data from the greater number of historical patients available as time progresses. For example, every year, every month, every week, or every day, an updated database of historical (past) patients can be used to retrain the set of models in active use by creating a training and testing dataset from the available past data, training the models on the training data, and testing them to provide quantitative assessment on the testing data as described here.

An example embodiment of the present invention is directed to one or more processors, which can be implemented using any conventional processing circuit and device or combination thereof, e.g., a Central Processing Unit (CPU) of a Personal Computer (PC) or other workstation processor, to execute code provided, e.g., on a hardware computer-readable medium including any conventional memory device, to perform any of the methods described herein, alone or in combination. For example, in an example embodiment, the circuitry interfaces with a patient population database, obtaining therefrom data, and executes an algorithm by which the circuitry generates prediction models, as described above. In an example embodiment, the circuitry generates the models in the form of further executables processable by the circuitry (or other circuitry) to predict onset of a disease (or diagnose a disease) based on respective datasets of a respective patient. In an alternative example embodiment, the algorithms are programmed in hardwired fashion in the circuitry, e.g., in the form of an application specific integrated circuit (ASIC). The one or more processors can be embodied in a server or user terminal or combination thereof. The user terminal can be embodied, for example, as a desktop, laptop, hand-held device, Personal Digital Assistant (PDA), television set-top Internet appliance, mobile telephone, smart phone, etc., or as a combination of one or more thereof. The memory device can include any conventional permanent and/or temporary memory circuits or combination thereof, a non-exhaustive list of which includes Random Access Memory (RAM), Read Only Memory (ROM), Compact Disks (CD), Digital Versatile Disk (DVD), and magnetic tape.

An example embodiment of the present invention is directed to one or more hardware computer-readable media, e.g., as described above, on which are stored instructions executable by a processor to perform the methods described herein.

An example embodiment of the present invention is directed to the described methods being executed by circuitry, such as that described above.

An example embodiment of the present invention is directed to a method, e.g., of a hardware component or machine, of transmitting instructions executable by a processor to perform the methods described herein.

### DETAILED DESCRIPTION OF FIGURES

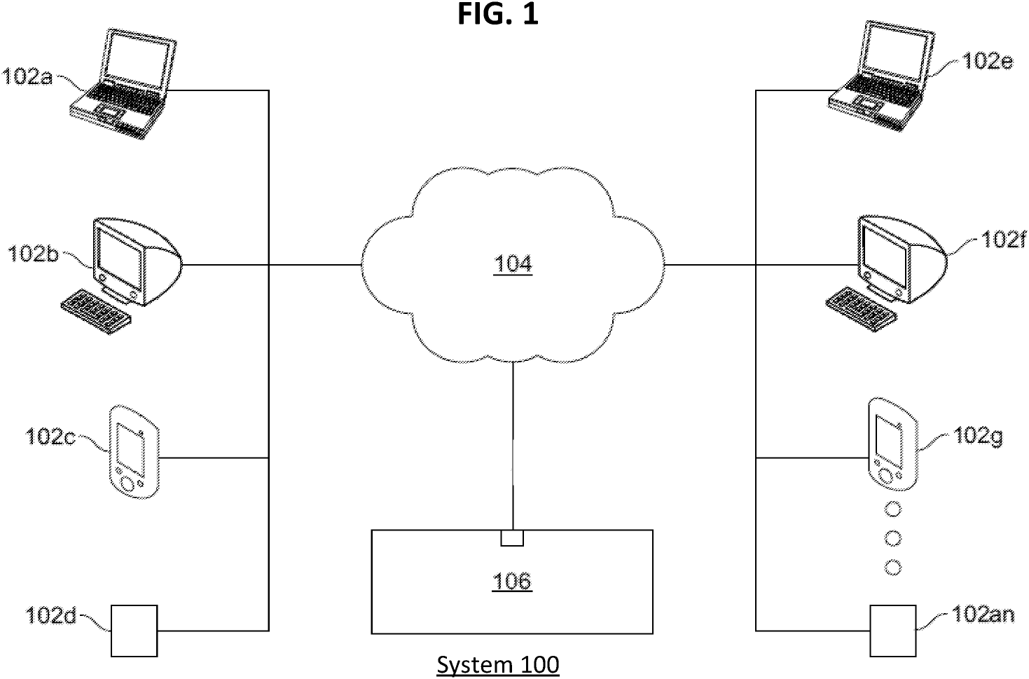

FIG. 1 illustrates an embodiment of the system utilized in the present disclosure. For example, as depicted in FIG. 1, system 100 includes a plurality of user terminals 102: laptops 102a and 102e, desktops 102b and 102f, hand-held devices 102c and 102g (e.g., smart phones, tablets, etc.), and other user terminals 102d and 102an. Further, in an embodiment, the other user terminals 102d and 102an can be any of a television set-top Internet appliance, mobile telephone, PDA, etc., or as a combination of one or more thereof. The system 100 also includes a communication network 104 and one or more processors 106. In an embodiment, the user terminals 102 interact with the one or more processors 106 via the communication network 104. In an embodiment, as discussed above, the processor 106 can be implemented using any conventional processing circuit and device or combination thereof, e.g., a Central Processing Unit (CPU) of a Personal Computer (PC) or other workstation processor or server, to execute code provided, e.g., on a hardware computer-readable medium including any conventional memory device, to perform any of the methods described herein, alone or in combination. For example, computational machine learning models running on one or more processors 106 can send predicted SIRS probabilities (or other predictions) to selected user terminals 102, 102a, 102b, 102c, etc. through the communication network 104. Users may choose to add notes, observations, or actions taken that are to be added to the patient data record by sending them through user terminals 102, 102a, 102b, 102c, etc. through the communication network 104 to one or more processors 106.

The above description is intended to be illustrative, and not restrictive. Those skilled in the art can appreciate from the foregoing description that the present invention may be implemented in a variety of forms, and that the various embodiments may be implemented alone or in combination. Therefore, while the embodiments of the present invention have been described in connection with particular examples thereof, the true scope of the embodiments and/or methods of the present invention should not be so limited since other modifications will become apparent to the skilled practitioner upon a study of the specification and following claims.

### PATENT REFERENCES

1. U.S. Pat. No. 7,645,573 (“the ‘573 patent”).

2. U.S. Pat. No. 8,029,982 (“the ‘982 patent”).

3. U.S. Pat. No. 8,527,449 (“the ‘449 patent”).

4. U.S. Pat. No. 8,697,370 (“the ‘370 patent”).

5. U.S. Pat. App. Pub. No. 2010/0190652 (“the ‘652 publication”).

6. U.S. Pat. App. Pub. No. 2013/0004968 (“the ‘968 publication”).

7. U.S. Pat. App. Pub. No. 2014/0248631 (“the ‘631 publication”).

8. U.S. Pat. App. Pub. No. 2015/0024969 (“the ‘969 publication”).

9. Int. Pat. App. Pub. No. WO 2013119869 (“the ‘869 publication”).

10. Int. Pat. App. Pub. No. WO 2014022530 (“the ‘530 publication”).

### NON-PATENT REFERENCES

1. Angus et al., Epidemiology of severe sepsis in the United States: Analysis of incidence, outcome, and associated costs of care, Crit Care Med. 29:1303-1310 (2001).

2. Angus et al., Severe sepsis and septic shock, N Engl J Med 2013; 369:840-851 (2013).

3. Annane et al., Septic shock, Lancet 365: 63-78 (2005).

4. Balci et al., Procalcitonin levels as an early marker in patients with multiple trauma under intensive care, J Int Med Res. 37:1709-17 (2009).

5. Barriere et al., An overview of mortality risk prediction in sepsis, Crit Care Med. 23:376-93 (1995).

6. Begley et al., Adding Intelligence to Medical Devices Medical Device & Diagnostic Industry Magazine (Mar. 1, 2000).

7. Bennett et al., Artificial intelligence framework for simulating clinical decision-making: A Markov decision process approach, Artificial Intelligence in Medicine, In Press (2013).

8. Bernstein, Transthyretin as a marker to predict outcome in critically ill patients, Clinical biochemistry 41:1126 -1130 (2008).

9. Bhattacharyya, On a measure of divergence between two statistical populations defined by their probability distributions, Bulletin of the Calcutta Mathematical Society, 35:99-109 (1943).

10. Bone et al., Definitions for sepsis and organ failure and guidelines for the use of innovative therapies in sepsis, Chest, 101:1644-1655 (1992).

11. Bracho-Riquelme et al., Leptin in sepsis: a well-suited biomarker in critically ill patients?, Crit Care. 14(2): 138 (2010).

12. Brandt et al., Identifying severe sepsis via electronic surveillance, Am J Med Qual. 30:559-65 (2015).

13. Brause et al., Septic shock diagnosis by neural networks and rule based systems, In: L.C. Jain (ed.), Computational Intelligence Techniques in Medical Diagnosis and Prognosis, Springer Verlag, New York, pp. 323-356 (2001).

14. Carrigan et al., Toward resolving the challenges of sepsis diagnosis, Clinical Chemistry 50:8, 1301-1314 (2004).

15. Comstedt et al., The Systemic inflammatory response syndrome (SIRS) in acutely hospitalised medical patients: a cohort study, Scand J Trauma Resusc Emerg Med. 17:67 (2009).

16. Dellinger et al., Surviving Sepsis Campaign: international guidelines for management of severe sepsis and septic shock: 2008, Crit Care Med. 36: 296-327 (2008).

17. Dellinger et al., Surviving Sepsis Campaign: international guidelines for management of severe sepsis and septic shock: 2012, Crit Care Med. 41:580-637 (2013).

18. Fialho et al., Predicting outcomes of septic shock patients using feature selection based on soft computing techniques, AMIA Annu Symp Proc. 653-662 (2012).

19. Giannoudis et al., Correlation between IL-6 levels and the systemic inflammatory response score: can an IL-6 cutoff predict a SIRS state?, J Trauma. 65:646-52 (2008).

20. Gultepe et al., From vital signs to clinical outcomes for patients with sepsis: a machine learning basis for a clinical decision support system, J Am Med Inform Assoc., 21:315-325 (2014).

21. Hanson et al., Artificial intelligence applications in the intensive care unit, Crit Care Med 29:427-434 (2001).

22. Hoeboer et al., Old and new biomarkers for predicting high and low risk microbial infection in critically ill patients with new onset fever: A case for procalcitonin, J Infect. 64:484-93 (2012).

23. Hohn et al., Procalcitonin-guided algorithm to reduce length of antibiotic therapy in patients with severe sepsis and septic shock, BMC Infect Dis. 13:158 (2013).

24. Hollenberg et al., Practice parameters for hemodynamic support of sepsis in adult patients: 2004 update, Crit Care Med. 32:1928-48 (2004).

25. Jekarl et al., Procalcitonin as a diagnostic marker and IL-6 as a prognostic marker for sepsis, Diagn Microbiol Infect Dis. 75:342-7 (2013).

26. Lai et al., Diagnostic value of procalcitonin for bacterial infection in elderly patients in the emergency department, J Am Geriatr Soc. 58:518-22 (2010).

27. Mani et al., Medical decision support using machine learning for early detection of late-onset neonatal sepsis, J Am Med Inform Assoc. 21: 326-336 (2014).

28. Marques et al., Preprocessing of Clinical Databases to improve classification accuracy of patient diagnosis, Preprints of the 18th IFAC World Congress Milano (Italy) (August 28 - Sep. 2, 2011).

29. Nachimuthu et al., Early Detection of Sepsis in the Emergency Department using Dynamic Bayesian Networks, AMIA Annu Symp Proc. 653-662 (2012).

30. Nierhaus et al., Revisiting the white blood cell count: immature granulocytes count as a diagnostic marker to discriminate between SIRS and sepsis--a prospective, observational study, BMC Immunol. 14:8 (2013).

31. Pittet et al., Systemic inflammatory response syndrome, sepsis, severe sepsis and septic shock: incidence, morbidities and outcomes in surgical ICU patients, Int Care Med. 21:302-309 (1995).

32. Pomi et al., Context-sensitive autoassociative memories as expert systems in medical diagnosis, BMC Medical Informatics and Decision Making, 6:39 (2006).

33. Rangel-Fausto et al., The natural history of the systemic inflammatory response syndrome (SIRS) A prospective study, JAMA 273:117-123 (1995).

34. Saeed et al., Multiparameter Intelligent Monitoring in Intensive Care II: A public-access intensive care unit database, Crit. Care Med., 39:952-960 (2011).

35. Selberg et al., Discrimination of sepsis and systemic inflammatory response syndrome by determination of circulating plasma concentration of procalcitonin, protein complement 3a and interleukin-6, Crit Care Med. 28:2793-2798 (2000).

36. Shapiro et al., The association of sepsis syndrome and organ dysfunction with mortality in emergency department patients with suspected infection, Ann Emerg Med. 48:583-590 (2006).

37. Sinning et al., Systemic inflammatory response syndrome predicts increased mortality in patients after transcatheter aortic valve implantation, Eur Heart J. 33:1459-68 (2012).

38. Tsoukalas et al., From data to optimal decision making: A data-driven, probabilistic machine learning approach to decision support for patients with sepsis, JMIR Med Inform 3(l):el 1 (2015).

39. Yu et al., Dual coordinate descent methods for logistic regression and maximum entropy models, Machine Learning 85:41-75 (2011).

What is claimed is:

1. A system for disease prediction, the system comprising:
processing circuitry including an interface, wherein the processing circuitry is configured to:
receive, via the interface, a dataset including data of a patient population, the data including for each of a plurality of patients of the patient population, values for a plurality of features and a diagnosis value of a diagnosis feature indicating whether a disease has been diagnosed;
based on correlations between the values, select from the dataset a plurality of subsets of the features; and
for each of at least one of the subsets:
execute a machine learning process with the respective subset and the diagnosis feature as input parameters, the execution generating a respective prediction model; and
output the respective prediction model.

2. The system of claim 1, wherein the selection of the plurality of subsets includes, for each of the plurality of subsets, (a) selecting a respective first one of the plurality of features as a primary feature based on a correlation of the respective first feature with the diagnosis feature, and (b) selecting a respective set of second ones of the plurality of features as secondary features based on a respective correlation of each of the respective second features with the respective first feature of the respective subset.

3. The system of claim 2, wherein, for each of the primary features, a feature is selected as a secondary feature of the respective primary feature conditional upon that the feature has a threshold level of correlation with the respective primary feature.

4. The system of claim 3, wherein the threshold level of correlation is 60% correlation.

5. The system of claim 2, wherein:
the selection of the plurality of subsets is performed iteratively, a respective one of the plurality of subsets being selected in each iteration; and
for each of the iterations, the subset selected in the respective iteration is removed from the dataset so that none of the features of the respective subset is selectable as a primary feature in any of the subsequent iterations and so that none of the features of the respective subset is selectable as a secondary feature in any of the subsequent iterations.

6. The system of claim 5, wherein:
the iterative selection includes, after each of the iterations:
applying the machine learning to a combination of all remaining features of the dataset; and
based on the application, determining whether the disease is predictable based on a prediction model whose parameters are values of the remaining features of the dataset; and
the iterative selection is ended in response to a negative result of the determination.

7. The system of claim 5, wherein:
the processing circuitry is further configured to divide the dataset into a training dataset and a testing dataset;
the machine learning process is executed based only on values of the training dataset; and
for each of the generated prediction models, the processing circuitry is configured to apply the generated prediction model to data of the testing dataset to determine a respective degree of prediction accuracy of the respective prediction model.

8. The system of claim 7, wherein the outputting is only of those of the generated prediction models for which the determined degree of prediction accuracy satisfies a predefined threshold.

9. The system of claim 5, wherein, in each iteration, whichever of the features remaining in the dataset has the strongest correlation with the diagnosis feature is selected as the primary feature of the respective subset.

10. The system of claim 9, wherein the processor is configured to, prior to the execution of the iterative selection:
for each of the features of the dataset, determine a distribution of values of the feature between entries that include a diagnosis value indicating that the disease has been diagnosed and entries that include a diagnosis value indicating that the disease has not been diagnosed; and
remove from the dataset all those entries whose distributions differ by less than a threshold amount, the iterative selection being performed only on those of the features remaining in the dataset after the removal.

11. The system of claim 5, wherein:
the dataset includes a plurality of datasets, each of the datasets corresponding to a respective onset time period, the diagnosis values of each of the datasets indicating whether the disease had been diagnosed within the respective time period to which the respective dataset corresponds;
the output prediction models include one or more prediction models for each of the onset time periods; and
each of the output prediction models, when executed, is configured to output a probability of onset of the disease within the onset time period to which the respective prediction model corresponds.

12. The system of claim 11, wherein:
the iterative selection results in selection of a subset for one of the onset time periods, which is not selected for another one of the onset time periods; and
a subset selected by the iterative selection for one of the onset time periods and not for another one of the onset time periods is applied as input even to the machine learning process whose output prediction model is associated with the onset time period for which the subset was not selected.

13. The system of claim 11, wherein the disease is Systemic Inflammatory Response Syndrome (SIRS) and the onset time periods are 6, 12, 24, and 48 hours.

14. The system of claim 5, wherein the disease is Systemic Inflammatory Response Syndrome (SIRS).

15. The system of claim 14, wherein the prediction model is a regression model.

16. The system of claim 15, wherein:
the model is
$$\begin{matrix}P \left(S I R S \left|{\text{patient\_data}}_{i}\right)\right) = \\ \frac{1}{1 + exp \left(- b - {\sum }_{j = 1}^{\text{num\_features}} {w}_{j} \times {\text{patient\_data}}_{i , j}\right)} ;\end{matrix}$$
P(SIRS|patient_dataᵢ) is a probability that a particular patient i, to which patient data represented by a vector patient_dataᵢ corresponds, will develop SIRS;
b is a model bias parameter;
num_features is the number of features in the respective subset of the model, indexed by j; and
wⱼ is a model coefficient for a respective one of the features j of the subset to which the model corresponds.

17. A computer-implemented method for disease prediction, the method comprising:
accessing, by processing circuitry, a dataset of a database, the dataset including data of a patient population, the data including for each of a plurality of patients of the patient population, values for a plurality of features and a diagnosis value of a diagnosis feature indicating whether a disease has been diagnosed;
based on correlations between the values, selecting, by the processing circuitry and from the dataset, a plurality of subsets of the features; and
for each of at least one of the subsets:
executing, by the processing circuitry, a machine learning process with the respective subset and the diagnosis feature as input parameters, the execution generating a respective prediction model; and
outputting, by the processing circuitry, the respective prediction model.

18. The method of claim 17, wherein the selection of the plurality of subsets includes, for each of the plurality of subsets, (a) selecting a respective first one of the plurality of features as a primary feature based on a correlation of the respective first feature with the diagnosis feature, and (b) selecting a respective set of second ones of the plurality of features as secondary features based on a respective correlation of each of the respective second features with the respective first feature of the respective subset.

19. The method of claim 18, wherein, for each of the primary features, a feature is selected as a secondary feature of the respective primary feature conditional upon that the feature has a threshold level of correlation with the respective primary feature.

20. The method of claim 19, wherein the threshold level of correlation is 60% correlation.

21. The method of claim 18, wherein:
the selection of the plurality of subsets is performed iteratively, a respective one of the plurality of subsets being selected in each iteration; and
for each of the iterations, the subset selected in the respective iteration is removed from the dataset so that none of the features of the respective subset is selectable as a primary feature in any of the subsequent iterations and so that none of the features of the respective subset is selectable as a secondary feature in any of the subsequent iterations.

22. The method of claim 21, wherein:
the iterative selection includes, after each of the iterations:
applying the machine learning to a combination of all remaining features of the dataset; and
based on the application, determining whether the disease is predictable based on a prediction model whose parameters are values of the remaining features of the dataset; and
the iterative selection is ended in response to a negative result of the determination.

23. The method of claim 21, further comprising:
dividing the dataset into a training dataset and a testing dataset, wherein the machine learning process is executed based only on values of the training dataset; and
for each of the generated prediction models, applying the generated prediction model to data of the testing dataset to determine a respective degree of prediction accuracy of the respective prediction model.

24. The method of claim 23, wherein the outputting is only of those of the generated prediction models for which the determined degree of prediction accuracy satisfies a predefined threshold.

25. The method of claim 21, wherein, in each iteration, whichever of the features remaining in the dataset has the strongest correlation with the diagnosis feature is selected as the primary feature of the respective subset.

26. The method of claim 25, further comprising, prior to the execution of the iterative selection:
for each of the features of the dataset, determining a distribution of values of the feature between entries that include a diagnosis value indicating that the disease has been diagnosed and entries that include a diagnosis value indicating that the disease has not been diagnosed; and
removing from the dataset all those entries whose distributions differ by less than a threshold amount, the iterative selection being performed only on those of the features remaining in the dataset after the removal.

27. The method of claim 21, wherein:
the dataset includes a plurality of datasets, each of the datasets corresponding to a respective onset time period, the diagnosis values of each of the datasets indicating whether the disease had been diagnosed within the respective time period to which the respective dataset corresponds;
the output prediction models include one or more prediction models for each of the onset time periods; and
each of the output prediction models, when executed, is configured to output a probability of onset of the disease within the onset time period to which the respective prediction model corresponds.

28. The method of claim 27, wherein:
the iterative selection results in selection of a subset for one of the onset time periods, which is not selected for another one of the onset time periods; and
a subset selected by the iterative selection for one of the onset time periods and not for another one of the onset time periods is applied as input even to the machine learning process whose output prediction model is associated with the onset time period for which the subset was not selected.

29. The method of claim 27, wherein the disease is Systemic Inflammatory Response Syndrome (SIRS) and the onset time periods are 6, 12, 24, and 48 hours.

30. The method of claim 21, wherein the disease is Systemic Inflammatory Response Syndrome (SIRS).

31. The method of claim 30, wherein the prediction model is a regression model.

32. The method of claim 31, wherein:
the model is
$$\begin{matrix}P ( S I R S \vert {\text{patient\_data}}_{i} ) = \\ \frac{1}{1 + exp ( - b - \sum {}_{j = 1}^{n u m \_ f e a t u r e s} {w}_{j} \times {\text{patient\_data}}_{i , j} )} ;\end{matrix}$$
P(SIRSlpatient_dataᵢ) is a probability that a particular patient i, to which patient data represented by a vector patient_dataᵢ corresponds, will develop SIRS;
b is a model bias parameter;
num_features is the number of features in the respective subset of the model, indexed by j; and
wⱼ is a model coefficient for a respective one of the features j of the subset to which the model corresponds.

33. A non-transitory computer-readable medium on which are stored instructions that are executable by a processor and that, when executed by the processor, cause the processor to perform a method for disease prediction, the method comprising:
accessing a dataset of a database, the dataset including data of a patient population, the data including for each of a plurality of patients of the patient population, values for a plurality of features and a diagnosis value of a diagnosis feature indicating whether a disease has been diagnosed;
based on correlations between the values, selecting from the dataset a plurality of subsets of the features; and
for each of at least one of the subsets:
executing a machine learning process with the respective subset and the diagnosis feature as input parameters, the execution generating a respective prediction model; and
outputting the respective prediction model.

34. The non-transitory computer-readable medium of claim 33, wherein the selection of the plurality of subsets includes, for each of the plurality of subsets, (a) selecting a respective first one of the plurality of features as a primary feature based on a correlation of the respective first feature with the diagnosis feature, and (b) selecting a respective set of second ones of the plurality of features as secondary features based on a respective correlation of each of the respective second features with the respective first feature of the respective subset.

35. The non-transitory computer-readable medium of claim 34, wherein, for each of the primary features, a feature is selected as a secondary feature of the respective primary feature conditional upon that the feature has a threshold level of correlation with the respective primary feature.

36. The non-transitory computer-readable medium of claim 35, wherein the threshold level of correlation is 60% correlation.

37. The non-transitory computer-readable medium of claim 34, wherein:
the selection of the plurality of subsets is performed iteratively, a respective one of the plurality of subsets being selected in each iteration; and
for each of the iterations, the subset selected in the respective iteration is removed from the dataset so that none of the features of the respective subset is selectable as a primary feature in any of the subsequent iterations and so that none of the features of the respective subset is selectable as a secondary feature in any of the subsequent iterations.

38. The non-transitory computer-readable medium of claim 36, wherein:
the iterative selection includes, after each of the iterations:
applying the machine learning to a combination of all remaining features of the dataset; and
based on the application, determining whether the disease is predictable based on a prediction model whose parameters are values of the remaining features of the dataset; and
the iterative selection is ended in response to a negative result of the determination.

39. The non-transitory computer-readable medium of claim 36, wherein the method further comprises:
dividing the dataset into a training dataset and a testing dataset, wherein the machine learning process is executed based only on values of the training dataset; and
for each of the generated prediction models, applying the generated prediction model to data of the testing dataset to determine a respective degree of prediction accuracy of the respective prediction model.

40. The non-transitory computer-readable medium of claim 39, wherein the outputting is only of those of the generated prediction models for which the determined degree of prediction accuracy satisfies a predefined threshold.

41. The non-transitory computer-readable medium of claim 36, wherein, in each iteration, whichever of the features remaining in the dataset has the strongest correlation with the diagnosis feature is selected as the primary feature of the respective subset.

42. The non-transitory computer-readable medium of claim 41, wherein the method further comprises, prior to the execution of the iterative selection:
for each of the features of the dataset, determining a distribution of values of the feature between entries that include a diagnosis value indicating that the disease has been diagnosed and entries that include a diagnosis value indicating that the disease has not been diagnosed; and
removing from the dataset all those entries whose distributions differ by less than a threshold amount, the iterative selection being performed only on those of the features remaining in the dataset after the removal.

43. The non-transitory computer-readable medium of claim 36, wherein:
the dataset includes a plurality of datasets, each of the datasets corresponding to a respective onset time period, the diagnosis values of each of the datasets indicating whether the disease had been diagnosed within the respective time period to which the respective dataset corresponds;
the output prediction models include one or more prediction models for each of the onset time periods; and
each of the output prediction models, when executed, is configured to output a probability of onset of the disease within the onset time period to which the respective prediction model corresponds.

44. The non-transitory computer-readable medium of claim 43, wherein:
the iterative selection results in selection of a subset for one of the onset time periods, which is not selected for another one of the onset time periods; and
a subset selected by the iterative selection for one of the onset time periods and not for another one of the onset time periods is applied as input even to the machine learning process whose output prediction model is associated with the onset time period for which the subset was not selected.

45. The non-transitory computer-readable medium of claim 43, wherein the disease is Systemic Inflammatory Response Syndrome (SIRS) and the onset time periods are 6, 12, 24, and 48 hours.

46. The non-transitory computer-readable medium of claim 36, wherein the disease is Systemic Inflammatory Response Syndrome (SIRS).

47. The non-transitory computer-readable medium of claim 46, wherein the prediction model is a regression model.

48. The non-transitory computer-readable medium of claim 47, wherein:
the model is
$$\begin{matrix}P ( S I R S \vert {\text{patient\_data}}_{i} ) = \\ \frac{1}{1 + exp ( - b - \sum {}_{j = 1}^{n u m \_ f e a t u r e s} {w}_{j} \times {\text{patient\_data}}_{i , j} )} ;\end{matrix}$$
P(SIRSlpatient_dataᵢ) is a probability that a particular patient i, to which patient data represented by a vector patient_dataᵢ corresponds, will develop SIRS;
b is a model bias parameter;
num_features is the number of features in the respective subset of the model, indexed by j; and
wⱼ is a model coefficient for a respective one of the features j of the subset to which the model corresponds.

49. A system for disease prediction, the system comprising:
processing circuitry including at least one interface, wherein the processing circuitry is configured to:
receive via the at least one interface a set of data associated with a particular patient;
based on the received set of data, select one of a plurality of prediction models;
execute the selected prediction model by populating parameters of the prediction model with values from the received set of data; and
output via the at least one interface a probability of the particular patient developing the disease within a particular time frame.

50. The system of claim 49, wherein different ones of the plurality of prediction models correspond to different groups of features, and the selection is from only those of the prediction models for each of the features of the groups of features of which the set of data includes a respective value.

51. The system of claim 49, wherein the disease is Systemic Inflammatory Response Syndrome (SIRS).

52. The system of claim 51, wherein:
the selected model is
$$\begin{matrix}P ( S I R S \vert {\text{patient\_data}}_{i} ) = \\ \frac{1}{1 + exp ( - b - \sum {}_{j = 1}^{n u m \_ f e a t u r e s} {w}_{j} \times {\text{patient\_data}}_{i , j} )} ;\end{matrix}$$
P(SIRSlpatient_dataᵢ) is a probability that the particular patient i, to which the set of data represented by a vector patient_dataᵢ corresponds, will develop SIRS;
b is a model bias parameter;
num_features is the number of features in the model, indexed by j; and
wⱼ is a model coefficient for a respective one of the features j of the features of the model.

53. The system of claim 49, wherein:
the selected model is
$$\begin{matrix}P \left(D I S E A S E \left|{\text{patient\_data}}_{i}\right)\right) = \\ \frac{1}{1 + exp \left(- b - {\sum }_{j = 1}^{\text{num\_features}} {w}_{j} \times {\text{patient\_data}}_{i , j}\right)} ;\end{matrix}$$
P(DISEASE|patient-dataᵢ) is a probability that the particular patient i, to which the set of data represented by a vector patient_dataᵢ corresponds, will develop the disease;
b is a model bias parameter;
num_features is the number of features in the model, indexed by j; and
wⱼ is a model coefficient for a respective one of the features j of the features of the model.

54. A computer-implemented method for disease prediction, the method comprising:
receiving, by processing circuitry, a set of data associated with a particular patient;
based on the received set of data, selecting, by the processing circuitry, one of a plurality of prediction models;
executing, by the processing circuitry, the selected prediction model by populating parameters of the prediction model with values from the received set of data; and
outputting, by the processing circuitry and via an output device, a probability of the particular patient developing the disease within a particular time frame.

55. The method of claim 54, wherein different ones of the plurality of prediction models correspond to different groups of features, and the selection is from only those of the prediction models for each of the features of the groups of features of which the set of data includes a respective value.

56. The method of claim 54, wherein the disease is Systemic Inflammatory Response Syndrome (SIRS).

57. The method of claim 56, wherein:
the selected model is
$$\begin{matrix}P ( S I R S \vert {\text{patient\_data}}_{i} ) = \\ \frac{1}{1 + exp ( - b - \sum {}_{j = 1}^{n u m \_ f e a t u r e s} {w}_{j} \times {\text{patient\_data}}_{i , j} )} ;\end{matrix}$$
P(SIRSlpatient_dataᵢ) is a probability that the particular patient i, to which the set of data represented by a vector patient_dataᵢ corresponds, will develop SIRS;
b is a model bias parameter;
num_features is the number of features in the model, indexed by j; and
wⱼ is a model coefficient for a respective one of the features j of the features of the model.

58. The method of claim 54, wherein:
the selected model is
$$\begin{matrix}P \left(D I S E A S E \left|{\text{patient\_data}}_{i}\right)\right) = \\ \frac{1}{1 + exp \left(- b - {\sum }_{j = 1}^{\text{num\_features}} {w}_{j} \times {\text{patient\_data}}_{i , j}\right)} ;\end{matrix}$$
P(DISEASE|patient-dataᵢ) is a probability that the particular patient i, to which the set of data represented by a vector patient_dataᵢ corresponds, will develop the disease;
b is a model bias parameter;
num_features is the number of features in the model, indexed by j; and
wⱼ is a model coefficient for a respective one of the features j of the features of the model.

59. A non-transitory computer-readable medium on which are stored instructions that are executable by a processor and that, when executed by the processor, cause the processor to perform a method for disease prediction, the method comprising:
receiving a set of data associated with a particular patient;
based on the received set of data, selecting one of a plurality of prediction models;
executing the selected prediction model by populating parameters of the prediction model with values from the received set of data; and
outputting, via an output device, a probability of the particular patient developing the disease within a particular time frame.

60. The non-transitory computer-readable medium of claim 59, wherein different ones of the plurality of prediction models correspond to different groups of features, and the selection is from only those of the prediction models for each of the features of the groups of features of which the set of data includes a respective value.

61. The non-transitory computer-readable medium of claim 59, wherein the disease is Systemic Inflammatory Response Syndrome (SIRS).

62. The non-transitory computer-readable medium of claim 61, wherein:
the selected model is
$$\begin{matrix}P ( S I R S \vert {\text{patient\_data}}_{i} ) = \\ \frac{1}{1 + exp ( - b - \sum {}_{j = 1}^{n u m \_ f e a t u r e s} {w}_{j} \times {\text{patient\_data}}_{i , j} )} ;\end{matrix}$$
P(SIRSlpatient_dataᵢ) is a probability that the particular patient i, to which the set of data represented by a vector patient_dataᵢ corresponds, will develop SIRS;
b is a model bias parameter;
num_features is the number of features in the model, indexed by j; and
wⱼ is a model coefficient for a respective one of the features j of the features of the model.

63. The non-transitory computer-readable medium of claim 59, wherein:
the selected model is
$$\begin{matrix}P \left(D I S E A S E \left|{\text{patient\_data}}_{i}\right)\right) = \\ \frac{1}{1 + exp \left(- b - {\sum }_{j = 1}^{\text{num\_features}} {w}_{j} \times {\text{patient\_data}}_{i , j}\right)} ;\end{matrix}$$
P(DISEASE|patient-dataᵢ) is a probability that the particular patient i, to which the set of data represented by a vector patient_dataᵢ corresponds, will develop the disease;
b is a model bias parameter;
num_features is the number of features in the model, indexed by j; and
wⱼ is a model coefficient for a respective one of the features j of the features of the model.

64. A system for predicting Systemic Inflammatory Response Syndrome (SIRS), the system comprising:
processing circuitry including at least one interface, wherein the processing circuitry is configured to:
receive via the at least one interface a set of data associated with a particular patient;
select a prediction model;
execute the selected prediction model by populating parameters of the prediction model with values from the received set of data; and
output via the at least one interface a probability of the particular patient developing SIRS within a particular time frame.

65. The system of claim 64, wherein all of the parameters of the prediction model that are populated by the set of data are Lactic Acid (0.5-2.0) and Lactic Acid.

66. The system of claim 64, wherein all of the parameters of the prediction model that are populated by the set of data are Blood Urea Nitrogen (BUN) and BUN (6-20).

67. The system of claim 64, wherein all of the parameters of the prediction model that are populated by the set of data are Platelets, Glucose (70-105), and Glucose.

68. The system of claim 64, wherein all of the parameters of the prediction model that are populated by the set of data are PO/Gastric In Total and PO Intake.

69. The system of claim 64, wherein all of the parameters of the prediction model that are populated by the set of data are PO2 and Arterial PaO2.

70. The system of claim 64, wherein all of the parameters of the prediction model that are populated by the set of data are Urine Out Total, 24-hr Total Out, IV Infusion In Total, and Urine Out Foley.

71. The system of claim 64, wherein all of the parameters of the prediction model that are populated by the set of data are INR (2-4 ref. range), Magnesium (1.6-2.6), Magnesium, and free Ca.

72. The system of claim 64, wherein all of the parameters of the prediction model that are populated by the set of data are Fibrinogen, GCS Total Glasgow Coma Scale, SAPS-I Simplified Acute Physiology Score, Overall SOFA (Sequen. Organ Failure) Score, Heart Rate, TCPCV Insp. Time Ventilation, Alk. Phosphate, D-Dimer (0-500), Gentamycin/Random, Phenobarbital, Vancomycin/Trough, Braden Score, Stool Out Fecal Bag, Urine Out Void, Dilaudid, and Ultrafiltrate Total.

73. The system of claim 64, wherein only IV Nutrition Total is a parameter of the prediction model that is populated by the set of data.

74. The system of claim 64, wherein only Tidal Volume (Observ.) Lung Vol. Displac. is a parameter of the prediction model that is populated by the set of data.

75. The system of claim 64, wherein only CPK/MB Blood Test is a parameter of the prediction model that is populated by the set of data.

76. The system of claim 64, wherein only Cerebral Drain L Ventricular Drain is a parameter of the prediction model that is populated by the set of data.

77. The system of claim 64, wherein only positive end respiratory pressure (PEEP) is a parameter of the prediction model that is populated by the set of data.

78. The system of claim 64, wherein only 24-hr Total In is a parameter of the prediction model that is populated by the set of data.

79. The system of claim 64, wherein only Gastric Out Total is a parameter of the prediction model that is populated by the set of data.

80. The system of claim 64, wherein only D5W 250.0 ml + 100 mcg/kg/min Nitroglycerine-k is a parameter of the prediction model that is populated by the set of data.

81. The system of claim 64, wherein only Tidal Volume (Set) is a parameter of the prediction model that is populated by the set of data.

82. The system of claim 64, wherein only Cholesterol (< 200) is a parameter of the prediction model that is populated by the set of data.

83. The system of claim 64, wherein only Fingerstick Glucose is a parameter of the prediction model that is populated by the set of data.

84. The system of claim 64, wherein only 0.9% Normal Saline 1000 ml is a parameter of the prediction model that is populated by the set of data.

85. The system of claim 64, wherein only Total Hourly Output is a parameter of the prediction model that is populated by the set of data.

86. The system of claim 64, wherein all of the parameters of the prediction model that are populated by the set of data are, as defined in MIMIC II database, chart 1162, chart 1528, chart 1531, chart 198, chart 682, chart 779, chart 781, chart 785, chart 811, chart 818, chart 828, io 102, io 133, io 97, lab 50017, lab 50019, totalbal 1, totalbal 16, totalbal 19, totalbal 2, totalbal 20, and totalbal 26.

87. The system of claim 64, wherein all of the parameters of the prediction model that are populated by the set of data are, as defined in MIMIC II database, chart 1162, chart 1529, chart 1531, chart 198, chart 20001, chart 682, chart 779, chart 781, chart 785, chart 818, chart 828, io 102, io 133, io 97, lab 50017, lab 50019, totalbal 1, totalbal 16, totalbal 18, totalbal 19, totalbal 20, and totalbal 26.

88. The system of claim 64, wherein all of the parameters of the prediction model that are populated by the set of data are, as defined in MIMIC II database, chart 1162, chart 1531, chart 20001, chart 20009, chart 682, chart 779, chart 781, chart 785, chart 811, chart 818, chart 828, io 102, io 133, io 55, io 97, lab 50017, lab 50019, totalbal 1, totalbal 16, totalbal 19, totalbal 20, and totalbal 26.

89. The system of claim 64, wherein all of the parameters of the prediction model that are populated by the set of data are, as defined in MIMIC II database, chart 1162, chart 1529, chart 1531, chart 20009, chart 211, chart 682, chart 779, chart 781, chart 785, chart 818, chart 828, io 102, io 133, io 97, lab 50017, lab 50019, totalbal 1, totalbal 16, totalbal 19, totalbal 2, and totalbal 26.

90. The system of claim 64, wherein all of the parameters of the prediction model that are populated by the set of data are, as defined in MIMIC II database, chart 1162, chart 1531, chart 211, chart 671, chart 682, chart 779, chart 781, chart 785, chart 811, chart 818, chart 828, io 102, io 133, io 97, lab 50017, lab 50019, totalbal 1, totalbal 16, totalbal 18, totalbal 19, totalbal 20, and totalbal 26.

91. The system of claim 64, wherein all of the parameters of the prediction model that are populated by the set of data are, as defined in MIMIC II database, chart 1162, chart 1529, chart 1531, chart 671, chart 682, chart 773, chart 779, chart 781, chart 785, chart 818, chart 828, io 102, io 133, io 55, io 97, lab 50017, lab 50019, totalbal 1, totalbal 16, totalbal 19, totalbal 20, and totalbal 26.

92. The system of claim 64, wherein all of the parameters of the prediction model that are populated by the set of data are, as defined in MIMIC II database, chart 1162, chart 1531, chart 682, chart 773, chart 779, chart 781, chart 785, chart 793, chart 811, chart 818, chart 828, io 102, io 133, io 97, lab 50017, lab 50019, totalbal 1, totalbal 16, totalbal 19, totalbal 2, totalbal 20, and totalbal 26.

93. The system of claim 64, wherein all of the parameters of the prediction model that are populated by the set of data are, as defined in MIMIC II database, chart 1162, chart 1529, chart 1531, chart 682, chart 779, chart 781, chart 785, chart 793, chart 809, chart 818, chart 828, io 102, io 133, io 97, lab 50017, lab 50019, totalbal 1, totalbal 16, totalbal 18, totalbal 19, totalbal 20, and totalbal 26.

94. The system of claim 64, wherein all of the parameters of the prediction model that are populated by the set of data are, as defined in MIMIC II database, chart 1162, chart 1531, chart 682, chart 779, chart 781, chart 785, chart 809, chart 811, chart 818, chart 826, chart 828, io 102, io 133, io 55, io 97, lab 50017, lab 50019, totalbal 1, totalbal 16, totalbal 19, totalbal 20, and totalbal 26.

95. The system of claim 64, wherein all of the parameters of the prediction model that are populated by the set of data are, as defined in MIMIC II database, chart 1162, chart 1531, chart 682, chart 779, chart 781, chart 785, chart 811, chart 818, chart 828, io 102, io 133, io 53, io 69, io 97, lab 50017, lab 50019, totalbal 1, totalbal 16, totalbal 19, totalbal 2, totalbal 20, and totalbal 26.

96. The system of claim 64, wherein all of the parameters of the prediction model that are populated by the set of data are, as defined in MIMIC II database, chart 811, chart 818, chart 1532, lab 50030, and totalbal 26.

97. The system of claim 64, wherein all of the parameters of the prediction model that are populated by the set of data are, as defined in MIMIC II database, lab 50030, io 55, io 97, totalbal 2, and totalbal 16.

98. The system of claim 64, wherein all of the parameters of the prediction model that are populated by the set of data are, as defined in MIMIC II database, chart 818, chart 1162, lab 50019, io 133, and totalbal 16.

99. The system of claim 64, wherein all of the parameters of the prediction model that are populated by the set of data are, as defined in MIMIC II database, chart 682, chart 1531, lab 50019, io 97, and totalbal 16.

100. The system of claim 64, wherein all of the parameters of the prediction model that are populated by the set of data are, as defined in MIMIC II database, lab 50019 and io 102.

101. The system of claim 64, wherein all of the parameters of the prediction model that are populated by the set of data are, as defined in MIMIC II database, lab 50017 and io 97.

102. The system of claim 64, wherein only lab 50019, as defined in MIMIC II database, is a parameter of the prediction model that is populated by the set of data.

103. The system of claim 64, wherein only lab chart 682, as defined in MIMIC II database, is a parameter of the prediction model that is populated by the set of data.

104. The system of claim 64, wherein only a single parameter of the prediction model is populated by the data set.

105. The system of claim 64, wherein at least one of the parameters is a measurement related to a blood glucose level.

106. The system of claim 64, wherein at least one of the parameters is a measurement related to oxygen saturation in blood.

107. A computer-implemented method for predicting Systemic Inflammatory Response Syndrome (SIRS), the method comprising:
receiving, by processing circuitry, a set of data associated with a particular patient;
selecting, by the processing circuitry, a prediction model;
executing, by the processing circuitry, the selected prediction model by populating parameters of the prediction model with values from the received set of data; and
outputting, by the processing circuitry and via an output device, a probability of the particular patient developing SIRS within a particular time frame.

108. The method of claim 107, wherein all of the parameters of the prediction model that are populated by the set of data are Lactic Acid (0.5-2.0) and Lactic Acid.

109. The method of claim 107, wherein all of the parameters of the prediction model that are populated by the set of data are Blood Urea Nitrogen (BUN) and BUN (6-20).

110. The method of claim 107, wherein all of the parameters of the prediction model that are populated by the set of data are Platelets, Glucose (70-105), and Glucose.

111. The method of claim 107, wherein all of the parameters of the prediction model that are populated by the set of data are PO/Gastric In Total and PO Intake.

112. The method of claim 107, wherein all of the parameters of the prediction model that are populated by the set of data are PO2 and Arterial PaO2.

113. The method of claim 107, wherein all of the parameters of the prediction model that are populated by the set of data are Urine Out Total, 24-hr Total Out, IV Infusion In Total, and Urine Out Foley.

114. The method of claim 107, wherein all of the parameters of the prediction model that are populated by the set of data are INR (2-4 ref. range), Magnesium (1.6-2.6), Magnesium, and free Ca.

115. The method of claim 107, wherein all of the parameters of the prediction model that are populated by the set of data are Fibrinogen, GCS Total Glasgow Coma Scale, SAPS-I Simplified Acute Physiology Score, Overall SOFA (Sequen. Organ Failure) Score, Heart Rate, TCPCV Insp. Time Ventilation, Alk. Phosphate, D-Dimer (0-500), Gentamycin/Random, Phenobarbital, Vancomycin/Trough, Braden Score, Stool Out Fecal Bag, Urine Out Void, Dilaudid, and Ultrafiltrate Total.

116. The method of claim 107, wherein only IV Nutrition Total is a parameter of the prediction model that is populated by the set of data.

117. The method of claim 107, wherein only Tidal Volume (Observ.) Lung Vol. Displac. is a parameter of the prediction model that is populated by the set of data.

118. The method of claim 107, wherein only CPK/MB Blood Test is a parameter of the prediction model that is populated by the set of data.

119. The method of claim 107, wherein only Cerebral Drain L Ventricular Drain is a parameter of the prediction model that is populated by the set of data.

120. The method of claim 107, wherein only positive end respiratory pressure (PEEP) is a parameter of the prediction model that is populated by the set of data.

121. The method of claim 107, wherein only 24-hr Total In is a parameter of the prediction model that is populated by the set of data.

122. The method of claim 107, wherein only Gastric Out Total is a parameter of the prediction model that is populated by the set of data.

123. The method of claim 107, wherein only D5W 250.0 ml + 100 mcg/kg/min Nitroglycerine-k is a parameter of the prediction model that is populated by the set of data.

124. The method of claim 107, wherein only Tidal Volume (Set) is a parameter of the prediction model that is populated by the set of data.

125. The method of claim 107, wherein only Cholesterol (< 200) is a parameter of the prediction model that is populated by the set of data.

126. The method of claim 107, wherein only Fingerstick Glucose is a parameter of the prediction model that is populated by the set of data.

127. The method of claim 107, wherein only 0.9% Normal Saline 1000 ml is a parameter of the prediction model that is populated by the set of data.

128. The method of claim 107, wherein only Total Hourly Output is a parameter of the prediction model that is populated by the set of data.

129. The method of claim 107, wherein all of the parameters of the prediction model that are populated by the set of data are, as defined in MIMIC II database, chart 1162, chart 1528, chart 1531, chart 198, chart 682, chart 779, chart 781, chart 785, chart 811, chart 818, chart 828, io 102, io 133, io 97, lab 50017, lab 50019, totalbal 1, totalbal 16, totalbal 19, totalbal 2, totalbal 20, and totalbal 26.

130. The method of claim 107, wherein all of the parameters of the prediction model that are populated by the set of data are, as defined in MIMIC II database, chart 1162, chart 1529, chart 1531, chart 198, chart 20001, chart 682, chart 779, chart 781, chart 785, chart 818, chart 828, io 102, io 133, io 97, lab 50017, lab 50019, totalbal 1, totalbal 16, totalbal 18, totalbal 19, totalbal 20, and totalbal 26.

131. The method of claim 107, wherein all of the parameters of the prediction model that are populated by the set of data are, as defined in MIMIC II database, chart 1162, chart 1531, chart 20001, chart 20009, chart 682, chart 779, chart 781, chart 785, chart 811, chart 818, chart 828, io 102, io 133, io 55, io 97, lab 50017, lab 50019, totalbal 1, totalbal 16, totalbal 19, totalbal 20, and totalbal 26.

132. The method of claim 107, wherein all of the parameters of the prediction model that are populated by the set of data are, as defined in MIMIC II database, chart 1162, chart 1529, chart 1531, chart 20009, chart 211, chart 682, chart 779, chart 781, chart 785, chart 818, chart 828, io 102, io 133, io 97, lab 50017, lab 50019, totalbal 1, totalbal 16, totalbal 19, totalbal 2, and totalbal 26.

133. The method of claim 107, wherein all of the parameters of the prediction model that are populated by the set of data are, as defined in MIMIC II database, chart 1162, chart 1531, chart 211, chart 671, chart 682, chart 779, chart 781, chart 785, chart 811, chart 818, chart 828, io 102, io 133, io 97, lab 50017, lab 50019, totalbal 1, totalbal 16, totalbal 18, totalbal 19, totalbal 20, and totalbal 26.

134. The method of claim 107, wherein all of the parameters of the prediction model that are populated by the set of data are, as defined in MIMIC II database, chart 1162, chart 1529, chart 1531, chart 671, chart 682, chart 773, chart 779, chart 781, chart 785, chart 818, chart 828, io 102, io 133, io 55, io 97, lab 50017, lab 50019, totalbal 1, totalbal 16, totalbal 19, totalbal 20, and totalbal 26.

135. The method of claim 107, wherein all of the parameters of the prediction model that are populated by the set of data are, as defined in MIMIC II database, chart 1162, chart 1531, chart 682, chart 773, chart 779, chart 781, chart 785, chart 793, chart 811, chart 818, chart 828, io 102, io 133, io 97, lab 50017, lab 50019, totalbal 1, totalbal 16, totalbal 19, totalbal 2, totalbal 20, and totalbal 26.

136. The method of claim 107, wherein all of the parameters of the prediction model that are populated by the set of data are, as defined in MIMIC II database, chart 1162, chart 1529, chart 1531, chart 682, chart 779, chart 781, chart 785, chart 793, chart 809, chart 818, chart 828, io 102, io 133, io 97, lab 50017, lab 50019, totalbal 1, totalbal 16, totalbal 18, totalbal 19, totalbal 20, and totalbal 26.

137. The method of claim 107, wherein all of the parameters of the prediction model that are populated by the set of data are, as defined in MIMIC II database, chart 1162, chart 1531, chart 682, chart 779, chart 781, chart 785, chart 809, chart 811, chart 818, chart 826, chart 828, io 102, io 133, io 55, io 97, lab 50017, lab 50019, totalbal 1, totalbal 16, totalbal 19, totalbal 20, and totalbal 26.

138. The method of claim 107, wherein all of the parameters of the prediction model that are populated by the set of data are, as defined in MIMIC II database, chart 1162, chart 1531, chart 682, chart 779, chart 781, chart 785, chart 811, chart 818, chart 828, io 102, io 133, io 53, io 69, io 97, lab 50017, lab 50019, totalbal 1, totalbal 16, totalbal 19, totalbal 2, totalbal 20, and totalbal 26.

139. The method of claim 107, wherein all of the parameters of the prediction model that are populated by the set of data are, as defined in MIMIC II database, chart 811, chart 818, chart 1532, lab 50030, and totalbal 26.

140. The method of claim 107, wherein all of the parameters of the prediction model that are populated by the set of data are, as defined in MIMIC II database, lab 50030, io 55, io 97, totalbal 2, and totalbal 16.

141. The method of claim 107, wherein all of the parameters of the prediction model that are populated by the set of data are, as defined in MIMIC II database, chart 818, chart 1162, lab 50019, io 133, and totalbal 16.

142. The method of claim 107, wherein all of the parameters of the prediction model that are populated by the set of data are, as defined in MIMIC II database, chart 682, chart 1531, lab 50019, io 97, and totalbal 16.

143. The method of claim 107, wherein all of the parameters of the prediction model that are populated by the set of data are, as defined in MIMIC II database, lab 50019 and io 102.

144. The method of claim 107, wherein all of the parameters of the prediction model that are populated by the set of data are, as defined in MIMIC II database, lab 50017 and io 97.

145. The method of claim 107, wherein only lab 50019, as defined in MIMIC II database, is a parameter of the prediction model that is populated by the set of data.

146. The method of claim 107, wherein only lab chart 682, as defined in MIMIC II database, is a parameter of the prediction model that is populated by the set of data.

147. The method of claim 107, wherein only a single parameter of the prediction model is populated by the data set.

148. The method of claim 107, wherein at least one of the parameters is a measurement related to a blood glucose level.

149. The method of claim 107, wherein at least one of the parameters is a measurement related to oxygen saturation in blood.

150. A non-transitory computer-readable medium on which are stored instructions that are executable by a processor and that, when executed by the processor, cause the processor to perform a method for predicting Systemic Inflammatory Response Syndrome (SIRS), the method comprising:
receiving a set of data associated with a particular patient;
selecting a prediction model;
executing the selected prediction model by populating parameters of the prediction model with values from the received set of data; and
outputting via an output device a probability of the particular patient developing SIRS within a particular time frame.

151. The non-transitory computer-readable medium of claim 150, wherein all of the parameters of the prediction model that are populated by the set of data are Lactic Acid (0.5-2.0) and Lactic Acid.

152. The non-transitory computer-readable medium of claim 150, wherein all of the parameters of the prediction model that are populated by the set of data are Blood Urea Nitrogen (BUN) and BUN (6-20).

153. The non-transitory computer-readable medium of claim 150, wherein all of the parameters of the prediction model that are populated by the set of data are Platelets, Glucose (70-105), and Glucose.

154. The non-transitory computer-readable medium of claim 150, wherein all of the parameters of the prediction model that are populated by the set of data are PO/Gastric In Total and PO Intake.

155. The non-transitory computer-readable medium of claim 150, wherein all of the parameters of the prediction model that are populated by the set of data are PO2 and Arterial PaO2.

156. The non-transitory computer-readable medium of claim 150, wherein all of the parameters of the prediction model that are populated by the set of data are Urine Out Total, 24-hr Total Out, IV Infusion In Total, and Urine Out Foley.

157. The non-transitory computer-readable medium of claim 150, wherein all of the parameters of the prediction model that are populated by the set of data are INR (2-4 ref. range), Magnesium (1.6-2.6), Magnesium, and free Ca.

158. The non-transitory computer-readable medium of claim 150, wherein all of the parameters of the prediction model that are populated by the set of data are Fibrinogen, GCS Total Glasgow Coma Scale, SAPS-I Simplified Acute Physiology Score, Overall SOFA (Sequen. Organ Failure) Score, Heart Rate, TCPCV Insp. Time Ventilation, Alk. Phosphate, D-Dimer (0-500), Gentamycin/Random, Phenobarbital, Vancomycin/Trough, Braden Score, Stool Out Fecal Bag, Urine Out Void, Dilaudid, and Ultrafiltrate Total.

159. The non-transitory computer-readable medium of claim 150, wherein only IV Nutrition Total is a parameter of the prediction model that is populated by the set of data.

160. The non-transitory computer-readable medium of claim 150, wherein only Tidal Volume (Observ.) Lung Vol. Displac. is a parameter of the prediction model that is populated by the set of data.

161. The non-transitory computer-readable medium of claim 150, wherein only CPK/MB Blood Test is a parameter of the prediction model that is populated by the set of data.

162. The non-transitory computer-readable medium of claim 150, wherein only Cerebral Drain L Ventricular Drain is a parameter of the prediction model that is populated by the set of data.

163. The non-transitory computer-readable medium of claim 150, wherein only positive end respiratory pressure (PEEP) is a parameter of the prediction model that is populated by the set of data.

164. The non-transitory computer-readable medium of claim 150, wherein only 24-hr Total In is a parameter of the prediction model that is populated by the set of data.

165. The non-transitory computer-readable medium of claim 150, wherein only Gastric Out Total is a parameter of the prediction model that is populated by the set of data.

166. The non-transitory computer-readable medium of claim 150, wherein only D5W 250.0 ml + 100 mcg/kg/min Nitroglycerine-k is a parameter of the prediction model that is populated by the set of data.

167. The non-transitory computer-readable medium of claim 150, wherein only Tidal Volume (Set) is a parameter of the prediction model that is populated by the set of data.

168. The non-transitory computer-readable medium of claim 150, wherein only Cholesterol (< 200) is a parameter of the prediction model that is populated by the set of data.

169. The non-transitory computer-readable medium of claim 150, wherein only Fingerstick Glucose is a parameter of the prediction model that is populated by the set of data.

170. The non-transitory computer-readable medium of claim 150, wherein only 0.9% Normal Saline 1000 ml is a parameter of the prediction model that is populated by the set of data.

171. The non-transitory computer-readable medium of claim 150, wherein only Total Hourly Output is a parameter of the prediction model that is populated by the set of data.

172. The non-transitory computer-readable medium of claim 150, wherein all of the parameters of the prediction model that are populated by the set of data are, as defined in MIMIC II database, chart 1162, chart 1528, chart 1531, chart 198, chart 682, chart 779, chart 781, chart 785, chart 811, chart 818, chart 828, io 102, io 133, io 97, lab 50017, lab 50019, totalbal 1, totalbal 16, totalbal 19, totalbal 2, totalbal 20, and totalbal 26.

173. The non-transitory computer-readable medium of claim 150, wherein all of the parameters of the prediction model that are populated by the set of data are, as defined in MIMIC II database, chart 1162, chart 1529, chart 1531, chart 198, chart 20001, chart 682, chart 779, chart 781, chart 785, chart 818, chart 828, io 102, io 133, io 97, lab 50017, lab 50019, totalbal 1, totalbal 16, totalbal 18, totalbal 19, totalbal 20, and totalbal 26.

174. The non-transitory computer-readable medium of claim 150, wherein all of the parameters of the prediction model that are populated by the set of data are, as defined in MIMIC II database, chart 1162, chart 1531, chart 20001, chart 20009, chart 682, chart 779, chart 781, chart 785, chart 811, chart 818, chart 828, io 102, io 133, io 55, io 97, lab 50017, lab 50019, totalbal 1, totalbal 16, totalbal 19, totalbal 20, and totalbal 26.

175. The non-transitory computer-readable medium of claim 150, wherein all of the parameters of the prediction model that are populated by the set of data are, as defined in MIMIC II database, chart 1162, chart 1529, chart 1531, chart 20009, chart 211, chart 682, chart 779, chart 781, chart 785, chart 818, chart 828, io 102, io 133, io 97, lab 50017, lab 50019, totalbal 1, totalbal 16, totalbal 19, totalbal 2, and totalbal 26.

176. The system of claim 150, wherein all of the parameters of the prediction model that are populated by the set of data are, as defined in MIMIC II database, chart 1162, chart 1531, chart 211, chart 671, chart 682, chart 779, chart 781, chart 785, chart 811, chart 818, chart 828, io 102, io 133, io 97, lab 50017, lab 50019, totalbal 1, totalbal 16, totalbal 18, totalbal 19, totalbal 20, and totalbal 26.

177. The non-transitory computer-readable medium of claim 150, wherein all of the parameters of the prediction model that are populated by the set of data are, as defined in MIMIC II database, chart 1162, chart 1529, chart 1531, chart 671, chart 682, chart 773, chart 779, chart 781, chart 785, chart 818, chart 828, io 102, io 133, io 55, io 97, lab 50017, lab 50019, totalbal 1, totalbal 16, totalbal 19, totalbal 20, and totalbal 26.

178. The non-transitory computer-readable medium of claim 150, wherein all of the parameters of the prediction model that are populated by the set of data are, as defined in MIMIC II database, chart 1162, chart 1531, chart 682, chart 773, chart 779, chart 781, chart 785, chart 793, chart 811, chart 818, chart 828, io 102, io 133, io 97, lab 50017, lab 50019, totalbal 1, totalbal 16, totalbal 19, totalbal 2, totalbal 20, and totalbal 26.

179. The non-transitory computer-readable medium of claim 150, wherein all of the parameters of the prediction model that are populated by the set of data are, as defined in MIMIC II database, chart 1162, chart 1529, chart 1531, chart 682, chart 779, chart 781, chart 785, chart 793, chart 809, chart 818, chart 828, io 102, io 133, io 97, lab 50017, lab 50019, totalbal 1, totalbal 16, totalbal 18, totalbal 19, totalbal 20, and totalbal 26.

180. The non-transitory computer-readable medium of claim 150, wherein all of the parameters of the prediction model that are populated by the set of data are, as defined in MIMIC II database, chart 1162, chart 1531, chart 682, chart 779, chart 781, chart 785, chart 809, chart 811, chart 818, chart 826, chart 828, io 102, io 133, io 55, io 97, lab 50017, lab 50019, totalbal 1, totalbal 16, totalbal 19, totalbal 20, and totalbal 26.

181. The non-transitory computer-readable medium of claim 150, wherein all of the parameters of the prediction model that are populated by the set of data are, as defined in MIMIC II database, chart 1162, chart 1531, chart 682, chart 779, chart 781, chart 785, chart 811, chart 818, chart 828, io 102, io 133, io 53, io 69, io 97, lab 50017, lab 50019, totalbal 1, totalbal 16, totalbal 19, totalbal 2, totalbal 20, and totalbal 26.

182. The non-transitory computer-readable medium of claim 150, wherein all of the parameters of the prediction model that are populated by the set of data are, as defined in MIMIC II database, chart 811, chart 818, chart 1532, lab 50030, and totalbal 26.

183. The non-transitory computer-readable medium of claim 150, wherein all of the parameters of the prediction model that are populated by the set of data are, as defined in MIMIC II database, lab 50030, io 55, io 97, totalbal 2, and totalbal 16.

184. The non-transitory computer-readable medium of claim 150, wherein all of the parameters of the prediction model that are populated by the set of data are, as defined in MIMIC II database, chart 818, chart 1162, lab 50019, io 133, and totalbal 16.

185. The non-transitory computer-readable medium of claim 150, wherein all of the parameters of the prediction model that are populated by the set of data are, as defined in MIMIC II database, chart 682, chart 1531, lab 50019, io 97, and totalbal 16.

186. The non-transitory computer-readable medium of claim 150, wherein all of the parameters of the prediction model that are populated by the set of data are, as defined in MIMIC II database, lab 50019 and io 102.

187. The non-transitory computer-readable medium of claim 150, wherein all of the parameters of the prediction model that are populated by the set of data are, as defined in MIMIC II database, lab 50017 and io 97.

188. The non-transitory computer-readable medium of claim 150, wherein only lab 50019, as defined in MIMIC II database, is a parameter of the prediction model that is populated by the set of data.

189. The non-transitory computer-readable medium of claim 150, wherein only lab chart 682, as defined in MIMIC II database, is a parameter of the prediction model that is populated by the set of data.

190. The non-transitory computer-readable medium of claim 150, wherein only a single parameter of the prediction model is populated by the data set.

191. The non-transitory computer-readable medium of claim 150, wherein at least one of the parameters is a measurement related to a blood glucose level.

192. The non-transitory computer-readable medium of claim 150, wherein at least one of the parameters is a measurement related to oxygen saturation in blood.
